(19) United States
(12) Patent Application Publication
(10) Pub. No.: US 2022/0145333 A1
(43) Pub. Date: May 12, 2022

(54) IMPROVED PROCESS FOR INTEGRATION OF DNA CONSTRUCTS USING RNA-GUIDED ENDONUCLEASES

(71) Applicant: Sorrento Therapeutics, Inc., San Diego, CA (US)

(72) Inventors: Beibei Ding, San Diego, CA (US); Wenzhong Guo, San Diego, CA (US); Yanliang Zhang, San Diego, CA (US)

(21) Appl. No.: 17/438,039

(22) PCT Filed: Mar. 11, 2020
(86) PCT No.: PCT/US2020/022056

§ 371 Date: Sep. 10, 2021

Related U.S. Application Data
(60) Provisional application No. 62/816,836, filed on Mar. 11, 2019.
(60) Provisional application No. 62/901,735, filed on Sep. 17, 2019.

(51) Int. Cl. C12N 15/90; C12N 15/11; C12N 9/22

(52) U.S. Cl. CPC C12N 15/907; C12N 2310/20; C12N 9/22; C12N 15/11

(57) ABSTRACT

There is disclosed an improved, safer and commercially efficient process for developing genetically engineered cells. More specifically, there is disclosed a process comprises introducing a donor DNA construct, a guide RNA, and an RNA-guided nuclease with the host cells to be transfected; and introducing the three components into the host cell. There is further disclosed a donor DNA construct designed for inserting a CAR (chimeric antigen receptor) into a defined genomic site of a host cell. Further, the present disclosure provides a host cell transfected with a CAR that lacks viral vectors that can present a safety concern. The disclosure provides for more efficient and more cost-effective process for engineering T cells to express CAR constructs.

### RELATED APPLICATIONS

This application is a 35 U.S.C. § 371 national phase application of PCT Application PCT/US2020/022056 filed Mar. 11, 2020; which claims priority to U.S. Provisional Patent Application No. 62/816,836, filed Mar. 11, 2019 and to U.S. Provisional Patent Application No. 62/901,735, filed Sep. 17, 2019, each of which are incorporated by reference in their entireties.

### TECHNICAL FIELD

The present disclosure provides methods and compositions for efficiently integrating a DNA sequence of interest into a target DNA molecule, such as a host genome, using an RNA-guided endonuclease such as a cas protein.

### BACKGROUND

Targeted integration of an exogenous DNA sequence into a genomic locus has been highly desired. CRISPR-Cas genome engineering is a fast and relatively simple way to knockout gene function, or precisely knock-in a DNA sequence for gene correction or gene tagging. Targeted gene knockout is achieved through generation of a double-strand break (DSB) in the DNA using Cas9 nuclease and guide RNA (gRNA). The DSB is then repaired, often imperfectly, by random insertions or deletions (indels), through the endogenous non-homologous end joining (NHEJ) repair pathway. For knock-in experiments, in addition to the Cas9 nuclease and gRNA, a DNA donor template is required and the DSB is repaired with the donor template typically through the homology-directed repair (HDR) pathway.

Knock-in using a donor template, either a single-stranded DNA (ssDNA) donor oligo or donor plasmid (dsDNA), has a relatively low efficiency, often in the 1-10% range. Therefore, successful HDR-mediated knock-in experiments require important design considerations and experimental optimization. Using single-stranded oligodeoxynucleotides (ssODNs) with short homology arms, several groups have achieved precise DNA editing such as SNP correction or epitope tag addition. A donor plasmid (dsDNA) is able to integrate much longer exogenous DNA, however efficiency is very low. Several groups used an AAV (viral) vector to provide HDR donor ssDNA and combined with CRISPR/Cas9 to achieve 40-60% gene knock-in efficiency. However, these methods still need to produce high titer AAV vectors which is time-consuming and needs to be compatible with cGMP production for clinical application.

A genome engineering tool has been developed based on the components of the type II prokaryotic CRISPR (Clustered Regularly Interspaced Short palindromic Repeats) adaptive immune system of some bacteria such as S. pyogenes. This multi-component system referred to as RNA-guided Cas nuclease system or more simply as CRISPR, involves a Cas endonuclease, coupled with a guide RNA molecule, that have the ability to create double-stranded breaks in genomic DNA at specific sequences that are targeted by the guide RNA. The RNA-guided Cas endonuclease has the ability to cleave the DNA where the RNA guide hybridizes to the genome sequence. Additionally, the Cas9 nuclease cuts the DNA only if a specific sequence known as protospacer adjacent motif (PAM) is present immediately downstream of the target sequence in the genome. The canonical PAM sequence in S. pyogenes is 5′-NGG-3′, where N refers to any nucleotide.

It has been demonstrated that the expression of a single chimeric crRNA:tracrRNA transcript, which normally is expressed as two different RNAs in the native type II CRISPR system, is sufficient to direct the Cas9 nuclease to sequence-specifically cleave target DNA sequences. In addition, several mutant forms of Cas9 nuclease have been developed. For instance, one mutant form of Cas9 nuclease functions as a nickase, generating a break in complementary strand of DNA rather than both strands as with the wild-type Cas9. This allows repair of the DNA template using a high-fidelity pathway rather than NHEJ, which prevents formation of indels at the targeted locus, and possibly other locations in the genome to reduce possible off-target/toxicity effects while maintaining ability to undergo homologous recombination. Paired nicking can reduce off-target activity by 50- to 1,500-fold in cell lines and to facilitate gene knockout in mouse zygote without losing on-target cleavage efficiency.

In addition, cas proteins have been isolated from a variety of bacteria and have been found to use different PAM sequences than S. pyogenes Cas9. In addition, some cas proteins such as Cas12a naturally use a single RNA guide—that is, they use a crRNA that hybridizes to a target sequence but do not use a tracrRNA.

Adoptive immunotherapy involves transfer of autologous antigen-specific cells generated ex vivo, is a promising strategy to treat viral infections and cancer. The cells used for adoptive immunotherapy can be generated either by expansion of antigen-specific cells or redirection of cells through genetic engineering.

CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T cell. CARs have successfully allowed T cells to be redirected against antigens expressed at the surface of tumor cells from various malignancies including lymphomas and solid tumors.

CAR (chimeric antigen receptor) cell immunotherapy, which involves removing T-cells from a patient's blood, adding a CAR through gene transfer, and infusing the genetically engineered cells back into the body, is one of the most promising methods in treating cancer. Currently, the gene transfer techniques include viral-based gene transfer methods using gamma-retroviral vectors or lentiviral vectors. To make GMP (FDA's required good manufacturing practice regulations) level viral-vector, the viral vector has to comply with clinical safety standards such as replication incompetence, low genotoxicity, and low immunogenicity. These conventional approaches have ease of use and reasonable expression, however they can give rise to secondary transformation events, e.g., unwanted blood cancers and other events resulting from viral genome integration into the T cells.

A review article (Ren and Zhao, Protein Cell 8(9):634-643, 2017) indicates that any use of CRISPR/Cas9 still involves the use of viral vector for a knocking in process to insert a CAR (chimeric antigen receptor) construct into a T cell genome. “Gene editing with CRISPR encoded by non-integrating virus, such as adenovirus and adenovirus-associated virus (AAV), has also been reported.” In addition, Ren et al., Clin. Cancer Res. 16:1300, published online 4 Nov. 2016 used a CD19 CAR construct and found that gene disruption in T cells is not very efficient with lentiviral and adenoviral CRISPR.

Recently dimeric antigen receptors or “DARs” have been described (WO 2019/173837). These engineered receptors include two polypeptide chains, one of which includes light chain antibody variable and constant regions and the other of which includes heavy chain antibody variable and constant regions as well as a transmembrane region and an intracellular region. Like CARs, DARs can be engineered to bind cancer cell surface antigens. Constructs encoding can be configured to express both polypeptides from a common promoter.

Although RNA-guided endonucleases, such as the Cas9/CRISPR system, appear to be an attractive approach for genetically engineering some mammalian cells, the use of Cas9/CRISPR in primary cells, in particular in T cells, is significantly more difficult because: (1) T-cells are adversely affected by the introduction of DNA in their cytoplasm: high rate of apoptosis is observed when transforming cells with DNA vectors: (2) the CRISPR system requires stable expression of Cas9 in the cells, however, prolonged expression of Cas9 in living cells may lead to chromosomal defects; and (3) the specificity of current RNA-guided endonuclease is determined only by sequences comprising 11 nucleotides (N12-20NGG, where NGG represents the PAM), which makes it very difficult to identify target sequences in desired loci that are unique in the genome. Other nucleases, in addition to Cas9, are Cas12a, zinc finger nucleases (ZFN) or TAL effector nucleases (TALEN).

The present disclosure aims to provide solutions to these limitations in order to efficiently implement RNA-guided endonuclease engineering in host cells such as T cells. There is a need in the art for safer transduction techniques for Chimeric Antigen Receptor constructs that do not include transduction with viral vectors but instead can use transfection techniques. This includes increasing CAR construct transfection efficiency, while avoiding the risk of having viral genes potentially expressed by the transduced cells that are administered to a patient. The present disclosure was made to address this need in the art.

### SUMMARY

The present disclosure provides an improved, safer, and commercially efficient process for developing genetically engineered and transduced cells, including cells for immunotherapy. More specifically, the disclosed process comprises introducing an RNA-guided endonuclease, a guide RNA, and a donor DNA construct into host cells, where the guide RNA is engineered to direct the cas protein with which it is complexed to a targeted site of the host genome. Cleavage of the genomic DNA at the target site by the RNA-guided endonuclease and subsequent repair of the double stranded break using the donor fragment that includes homology arms by homology-directed repair (HDR) results in integration of sequences of the donor DNA molecule positioned between the homology arms. The method can be used to simultaneously knock out a gene at the target locus and insert or “knock in” at the disrupted locus a transgene that is provided in the donor DNA molecule. Further provided are methods for inserting a genetic construct at a first genetic locus, where insertion of the genetic construct knocks out a gene at the first locus, and simultaneously knocking out a gene at a second locus. The knockin/double knock out is achieved by introducing two RNPs into the target cell, a first RNP having a guide targeting the first genetic locus, and a second RNP having a guide targeting the second genetic locus. The two RNPs can comprise the same (e.g., Cas12a) or different (e.g., Cas12a and Cas9) cas proteins. The method can be used on any host cells, including prokaryotic and eukaryotic cells, and can be used with mammalian cells, such as human cells. The method has advantages in ease of use, efficiency, and the ability to generate genome modifications that do not entail the use of selectable markers or viral vectors that are undesirable in many applications, including clinical applications. In some embodiments, the host cells are hematopoietic cells, such as, for example, T cells.

The present disclosure also provides systems for targeted integration of a donor DNA into a locus of the genome of a eukaryotic cell. Also provided are donor DNA compositions, where the donor DNA molecule includes one or more modifications to nucleotides of one donor DNA strand. The donor DNA can include homology arms flanking a sequence of interest whose integration into the host genome is desired, where the homology arms have sequences homologous to sequences occurring in the host genome on either side of the target sequence. The donor DNA in some embodiments is double-stranded, or substantially double-stranded. In various embodiments the donor DNA includes from one to ten modified nucleotides that are proximal to the 5′ end of one strand of the donor DNA, for example, that occur within ten nucleotides or within five nucleotides of the 5′ terminus of one strand of the donor DNA. In some embodiments the donor DNA has at least two types of nucleic acid modification of from one to ten nucleotides at the 5′ end of one strand of the donor DNA. In some embodiments the donor DNA has two types of nucleic acid modification of from one to ten nucleotides at the 5′ end of one strand of the donor DNA. The modification may be, for example, phosphorothioate (PS) linkages between nucleotides, or may be 2′-O-methylation of the deoxyribose of one or more nucleotides of the donor DNA molecule. For example, a donor DNA molecule can have one, two, three or four PS bonds within the first five, first six, or first seven nucleotides from the 5′ end of the modified strand and can also have one, two, three or four 2′-O-methyl modified nucleotides within the first five, first six, or first seven nucleotides from the 5′ end of the modified strand. In some embodiments the donor DNA molecule is double-stranded and one strand comprises the modifications at the 5′ end. In some embodiments the donor DNA molecule is double-stranded and one strand has two or more modifications on any of the first ten or first five nucleotides from the 5′ end and the opposite strand has a terminal 5′ phosphate. In various embodiments, the donor DNA molecule is double-stranded and has at least two PS bonds and at least two 2′O-methyl-modified nucleotides on one strand of the donor DNA, where the PS and 2′-0 methyl modifications occur within the first five nucleotides from the 5′ end of the modified strand. In various embodiments, the donor DNA molecule is double-stranded or substantially double-stranded and has three PS bonds and three 2′O-methyl-modified nucleotides on one strand of the donor DNA, where the PS and 2′-O methyl modifications occur within the first five nucleotides from the 5′ end of the modified strand. In some examples of these embodiments, the opposite strand includes a terminal 5′ phosphate. The donor DNA can be introduced into the cell as a double-stranded or substantially double-stranded molecule.

The present disclosure further provides a donor DNA construct designed for inserting a CAR (chimeric antigen receptor) or DAR (dimeric antigen receptor) into a host cell. CAR constructs are well-known in the art and reviewed, for example, in Zhang et al. (2017) Biomarker Res. 5:22. DAR constructs, that encode a two polypeptide receptor, are described for example in WO 2019/173837. Further, the present disclosure provides a host cell transduced with a CAR that lacks a viral vector or component thereof, such as sequences of a retroviral or adeno-associated viral (AAV) vector. The disclosure provides for more efficient and more cost-effective process for engineering T cells to express CAR or DAR constructs. The CAR or DAR construct can include homology arms that target the construct to a T cell receptor gene, PD-1 gene, CD7 gene, or TIM3 gene, as nonlimiting examples, for simultaneous knock-in of the CAR construct and knock out of the TCR, PD-1, TIM3, GM-CSF, CD7, or other gene.

In a further aspect, provided herein is a system for genome modification that comprises: at least one RNA-guided endonuclease or at least one nucleic acid molecule encoding an RNA-guide endonuclease; at least one guide RNA or at least one nucleic acid molecule encoding a guide RNA; and a donor DNA molecule, where the donor DNA molecule includes at least one nucleotide modification within twenty, within ten, or within five nucleotides of the 5′ terminus. In some embodiments the donor DNA is double-stranded or substantially double-stranded and includes at least one, at least two, or at least three modifications on at least one, at least two, or at least three nucleotides occurring within ten or within five nucleotides of one strand of the double stranded donor molecule. The modifications can be, for example, backbone modifications such as phosphorothioate bonds and/or 2′-O methylation of the sugar of nucleotides. The donor DNA can be at least 250 nt or bp in length, and can be at least 300, 400, 500, 600, 700, 800, 900, or 1000 nt or bp in length, and in some embodiments can be greater than 2000 nt or bp in length, for example, may be between about 0.5 and 4 kb in length, or between about 1 kb and 3.5 kb in length, or between about 1.5 kb and about 2.8 kb in length, or between about 1.8 kb and about 3 kb in length, as nonlimiting examples. The donor DNA can have homology arms (HAs) flanking a sequence of interest to be integrated into the genome. The sequence of interest can be an expression cassette, for example, for expression a construct that includes one or more antibody or receptor domains. Homology arms can be between about 50 and about 5000 nucleotides in length, or between about 100 and 1000 nucleotides in length, for example between about 120 and about 800 nucleotides in length, or between about 140 and about 600 nucleotides in length.

In some embodiments, an RNA-guide nuclease used in the systems and methods provided herein is selected from the group consisting of Cas9, Cas12a, CasX, and combinations thereof. The guide RNA can be a chimeric guide, having sequences of both crRNA and tracrRNA, or can be a crRNA, and can optionally include one or more nucleic acid modifications, including phosphorothioate (PS) oligonucleotides. Where the guide is a crRNA, and the RNA-guided endonuclease uses a tracrRNA, the system can also include a tracrRNA. For example, Cas9 can be used with a crRNA and a tracrRNA or can be used with a chimeric guide RNA (sometimes called a single guide or “sgRNA”) that combines structural features of the crRNA and tracrRNA. Cas12a on the other hand naturally uses only a crRNA and has no associated tracrRNA. In various embodiments, the RNA-guide endonuclease, guide RNA (that can be a crRNA or a chimeric guide RNA), and, when included, tracr RNA, can be complexed as a ribonucleoprotein complex that is introduced to the cell. The donor DNA can be introduced into the target cell together with the RNP, or separately, for example, in a separate electroporation or transfection.

Also provided herein is a method for site-specific integration of a donor DNA into a target DNA molecule, where the method includes introducing into a cell: at least one RNA-guided endonuclease or a nucleic acid molecule encoding an RNA-guided endonuclease; at least one engineered guide RNA or at least one nucleic acid molecule encoding an engineered guide RNA; and a donor DNA molecule comprising at least one nucleic acid modification; where the guide RNA comprises a target sequence designed to hybridize with a target site sequence in the target DNA and the donor DNA is inserted into the target DNA molecule at the target site. The donor DNA can be, for example, at least 250 nucleotides or base pairs in length, at least 500 nucleotides or base pairs, at least 1000 nucleotides or base pairs, at least 1500 nucleotides or base pairs, at least 2000 nucleotides or base pairs, at least 2500 nucleotides or base pairs, or at least 3000 nucleotides or base pairs (bp) in length, where the donor fragment can be delivered to the cells as a double-stranded or substantially double-stranded molecule. In some embodiments the RNA-guided endonuclease is introduced into the cell as a protein. In some embodiments the guide RNA is introduced into the cell as an RNA molecule. In exemplary embodiments, the RNA-guided endonuclease, guide RNA, and, where employed, tracrRNA, are introduced into the cell as a ribonucleoprotein complex (RNP). In various examples the RNA-guided endonuclease is a Cas12a endonuclease and is delivered to the cell (for example by electroporation or liposome delivery) as an RNP complexed with the guide RNA, which in the case of Cas12a, is a crRNA.

Further provided are methods for site site-specific integration of a donor DNA into a first target locus combined with targeted knockout of a second target locus. The knock in/knock out at a first locus and knock out of a second locus can be performed by means of a single transfection event that introduces the donor DNA, RNA-guided endonuclease and guide targeting the first locus, and RNA-guided endonuclease and guide targeting the second locus in one transformation. The methods include simultaneously introducing into a cell: a first RNA-guided endonuclease complexed with a first engineered guide RNA targeting a first locus; a second RNA-guided endonuclease complexed with a second engineered guide RNA targeting a second locus; and a donor DNA molecule; where the first guide RNA comprises a target sequence designed to hybridize with a first target site in the target DNA and the donor DNA is is inserted into the target DNA molecule at the first target site, and the second locus is disrupted by modification by the second RNA-guided endonuclease. The donor DNA can have at least one, at least two, at least three nucleic acid modifications and can be, for example, at least 250 nucleotides or base pairs in length, at least 500 nucleotides or base pairs, at least 1000 nucleotides or base pairs, at least 1500 nucleotides or base pairs, at least 2000 nucleotides or base pairs, at least 2500 nucleotides or base pairs, or at least 3000 nucleotides or base pairs (bp) in length, where the donor fragment can be delivered to the cells as a double-stranded or substantially double-stranded molecule. The donor DNA has homology arms flanking a sequence of interest, such as a construct for expressing a gene, where the homology arms have homology to sequences proximal to the first target site in the host genome. The second locus is preferably a site within a gene whose knockout is desired. Incorporation of the donor DNA into the first locus and knockout of the second locus can occur in at least 10%, at least 20%, at least 30%, at least 40%, at least 50%, or at least 60% of the transfected cell population.

Provided herein are methods for genetically modifying mammalian cells, such as human cells, such as primary human T cells, at two different genetic loci by delivering to the cells two RNPs: a first RNP that includes a guide RNA for targeting a first genetic locus and a second RNP that includes a guide RNA for targeting a second genetic locus. The first and second RNPs can comprise the same or different cas proteins and may be delivered to the cell simultaneously or sequentially. For example, a first RNP can comprise cas9 protein and a second RNP can comprise cas12a protein, or vice versa. Alternatively, both the first and second RNPs can comprise cas12a protein. A donor DNA having homology arms for the first genetic locus can also be delivered to the cell, simultaneously with the first RNP or at a later or earlier time. In embodiments where a donor DNA is delivered, the methods can include insertion of a non-native genetic construct to a first genetic locus of the cell, where the first genetic locus can be disrupted by the insertion of the non-native genetic construct, and cas-mediated disruption of a second genetic locus of the cell targeted by the second RNP. Cells modified by these methods can express a non-native construct, such as but not limited to a CAR or DAR construct, and can exhibit reduced expression of endogenous genes at the first and second genetic loci. For example, the first and second genetic loci can be mutated by means of the cas proteins and complexed guide RNAs delivered to the cell. The genes at the first and second loci can be disrupted to greatly reduce or eliminate (knock out) gene expression. The donor DNA can be any donor DNA as provided herein, such as a double-stranded donor DNA having at least two nucleic acid modifications to at least one strand. The donor DNA preferably has homology arms that comprise sequences of the first genetic locus that flank the genetic construct (e.g., CAR or DAR encoding sequence).

In various embodiments of the systems, compositions, and methods provided herein the donor DNA includes at least two modified nucleotides, which can have the same or different modifications, and preferably occur within ten or within five nucleotides of the 5′ terminus of one strand of the donor DNA. In some embodiments, the donor DNA is double-stranded and the one or more nucleotide modifications occur on a single strand of the donor DNA molecule. In some embodiments, the donor DNA is double-stranded and the one or more nucleotide modifications occur on a single strand of the donor DNA molecule within twenty, within ten, or within five nucleotides of the 5′ terminus of the modified strand. In some embodiments, the donor DNA includes a backbone modification such as a phosphoramidite or phosphorothioate modification. In some embodiments, the donor DNA includes a modification of a sugar moiety of a nucleotide. In some embodiments, the donor DNA is double stranded and includes at least one, at least two, or at least three phosphorothioate modifications within five nucleotides of the 5′ end of a single strand of the donor DNA molecule and further includes at least one, at least two, or at least three 2′-O-methylated nucleotides within five nucleotides of the 5′ end of a single strand of the donor DNA molecule. In various embodiments the donor DNA includes homology arms flanking a DNA sequence of interest, such as, for example, an expression cassette, where the homology arms have homology to sites in the target genome on either side of the target site of the RNA-guide endonuclease. Homology arms can be from about 50 to about 2000 nt in length, and may be, for example between 100 and 1000 nt in length, or between 150 and 650 nt in length, for example, between 150 and 350 nt in length, or 150 to 200 nt in length. In various embodiments a donor DNA molecule has two or more nucleotide modifications on the modified strand and the opposite strand includes a terminal phosphate.

The RNA-guided endonuclease can be a cas protein and can be, as nonlimiting examples, a Cas9, Cas12a, or CasX protein. In various embodiments of the method, the at least one RNA-guided endonuclease and at least one RNA guide are introduced into the cell as one or more ribonucleoprotein complexes (RNPs). In various embodiments a first RNP is formed with a cas protein and a first guide RNA and a second RNP is formed in a separate incubation of a cas protein and a second guide RNA. The cas protein for each RNP can be the same or different. For example, a first RNP can be formed with cas9, and a second RNP can be formed with cas12a. One or more RNPs that includes a cas9 protein can in some embodiments further include a tracrRNA. The two RNPs, and, optionally a donor DNA, can be added to the cells for multiple site gene editing, where at least one of the edited sites optionally incorporates a DNA donor. An RNP can be introduced into a target cell by any feasible means, including electroporation or liposome transfer, for example. The donor DNA can be delivered to the cell simultaneously with the one or more RNPs, or separately.

The methods can be used to modify the genomes of eukaryotic cells, including the cells of animals, including avian, fish, insect, and mammalian cells. In various embodiments the cells whose genomes are manipulated using the methods and systems provided herein are mammalian cells and may be human cells. Cells used in the methods provided herein can be of cell lines or can be primary cells, such as, for example, stem cells or hematopoietic cells, including T cells and NK cells.

Further included herein are engineered primary T cells, which may be human primary T cells, where the cells include a non-native genetic construct integrated into the genome at a first genetic locus that comprises a first target site of an RNA-guided nuclease and a mutation at a second genetic locus that comprises a second target site of an RNA-guided nuclease. The mutation at the second genomic locus can be, for example, a knockout mutation by means of an insertion or deletion inserted at the second target site as a result of cas nuclease activity and misrepair by the cell. The second target site may be in a gene whose reduced expression is desired. The first target site, into which the donor fragment that comprises the non-native genetic construct is inserted, may also be in a gene whose reduced expression is desired. “Target site” as used herein means a sequence adjacent to a PAM sequence recognized by an RNA-guided nuclease. Such PAM-adjacent sequences (of, for example 17-22 nucleotides in length) can be used as target sequences in guide RNAs to direct the activity of a cas nuclease such as cas9 or cas12a to cleave the genomic DNA the target site.

The non-native genetic construct is a genetic construct that does not naturally occur in the cells that is introduced on a donor fragment for integration using the cas-mediated methods provided herein. The engineered primary T cells can express the non-native genetic construct and can have reduced expression of the gene at the second genetic locus, and can also have reduced expression of the gene at the first genetic locus, where the gene at the first genetic locus may be disrupted by insertion of the non-native genetic construct.

The non-native genetic construct can be a genetic construct that encodes one or more polypeptides having one or more immunoglobulin domains. In some embodiments, the non-native genetic construct is construct that encodes a CAR or DAR. Thus, in some embodiments primary human T cells are provided that include a non-native genetic construct such as a CAR or DAR-encoding construct integrated into the genome, where the cells express the construct (e.g., express a CAR or DAR molecule) and may have reduced expression of a gene disrupted by insertion of the CAR or DAR (or other) construct, and where the cells can have a second site mutation that results in reduced expression of a second gene. Genes that may be disrupted by insertion of a genetic construct include, without limitation, genes encoding the TCR, TRAC, PD-1, CTL4-A, TIM3, LAG3, GM-CSF, and CD7. A CAR or DAR can be a CAR or DAR designed to bind a tumor cell surface antigen, such as but not limited to, BCMA, CD19, CD20, CD38, CD123, or any other tumor cell surface antigen. In various examples, at least 25% of a population of cells as provided herein may express a CAR, DAR, or other introduced construct and exhibit reduced expression of a gene at a second genetic locus. In various examples, at least 25% of a population of cells as provided herein may express a CAR, DAR, or other introduced construct and exhibit reduced expression of a gene at the first genetic locus into which the non-native construct have been introduced and exhibit reduced expression of a gene at the second genetic locus. The cells may be produced using the methods provided herein.

Provided in yet another aspect are human primary T cells having a CAR or DAR construct inserted into the CD7 gene, as demonstrated herein. A population of T cells having a CAR or DAR insertion in the CD7 gene can demonstrate CAR or DAR expression and reduced expression of CD7.

### BRIEF DESCRIPTION OF THE FIGURES

FIG. 2A provides chemical drawings that show, in the right structure, a phosphorothioate (PS) modification of the bond between nucleotides as they might occur in a primer. The nucleotides shown in the oligonucleotide on the left are attached via a (nonmodified) phosphodiester bond. FIG. 2B provides a chemical drawing of an oligonucleotide having two PS bonds that join the 5′-most nucleotide to the next nucleotide “downstream” in the oligonucleotide, which in turn is attached to the following downstream nucleotide of the oligonucleotide by a PS bond. The 5′-most nucleotide of the oligonucleotide includes a 2′ O-methyl modification.

FIG. 1A is a diagram of a CAR donor DNA construct that includes an open reading frame having a sequence encoding a single chain variable fragment (scFv), followed by the CD8a leader peptide which is then followed by a CD28 hinge-CD28 transmembrane-intracellular regions and then a CD3 zeta intracellular domain. The coding sequence is preceded by a JeT promoter (SEQ ID NO:3) and the construct includes homology arms (HAs), in this case matching sequences of the human TRAC locus, flanking the promoter plus coding sequences. shows the structure of the donor DNA construct (top) and primer design for confirming right knock in (bottom). This provides a diagram of the template DNA used for generating donor DNA. The anti-CD38A2 contains a CD38 CAR transgene with expression driven by the JeT promoter and flanked by homology arms on the 5′ and 3′ sides to enable targeted integration.

FIG. 1B shows the same diagram indicating the positions of PCR primers used to confirm CAR integration by amplification with one primer located within the CAR and one primer in TRAC outside of the homology arms at both the 5′ and 3′ ends to generate 1371-bp and 1591-bp products, respectively, when integration is at the targeted integration site.

FIGS. 3A to 3E provide flow cytometry plots of PBMCs 8 days after transformation with a donor DNA that included a construct for expressing an anti-CD38 CAR and an RNP comprising a guide RNA targeting the TRAC locus. The CAR cassette was flanked by homology arms having homology to TRAC locus sequences flanking the integration target site in exon 1 of the TRAC gene. The Y axis reports cell size. Anti-CD38 construct expression is along the x axis. Negative control: no donor DNA was transformed into the target cells; No modification—the donor DNA had no chemical modifications; PS modification: three phosphorothioate bonds occurred within the 5′-most five nucleotide backbone positions; PS+2′-OMe: in addition to phosphorothioate bonds, the three nucleotides within the 5′-most five nucleotides of the donor included 2′-OMe in addition to PS modifications; TCR KO/retroviral construct: the cells were transfected with the RNP in the absence of donor DNA to knock out the TCR gene and transduced with a retrovirus to express the anti-CD38 CAR.

FIGS. 3F to 3J provide the results of flow cytometry performed on the same cultures as in A) ten days after transfection.

FIGS. 3K to 3M provide the results of flow cytometry performed on the culture that received the doubly-modified donor DNA and control (TRAC knockout only and TRAC knockout with retroviral transduction) twenty days after transfection.

FIG. 4 shows a gel of PCR products showing integration of the donor DNA at the targeted TRAC (Exon1) site. Primary human T cells were electroporated with TRAC RNP only or together with ssDNA. PCR was used to confirm the presence of the anti-CD38A2 CAR transgene integrated in the TRAC locus two weeks post-electroporation (lanes 3 and 6, depicting products from 5′ and 3′ integration regions). No bands were observed in non-transformed ATCs (lanes 1 and 4) or T cells that were transformed with the TRAC exon 1 targeting RNP but did not receive the donor DNA (lanes 2 and 5).

FIG. 5 is a graph showing cytotoxicity assay results with Activated T cells (ATCs, stars) as a control, TCR knock out ATC, anti-CD38A2 retrovirus transduced CART cells RV CART, black line), TRAC knock out retrovirus transduced CART cells (dots), TRAC knock out together with phosphorothioate modified ss donor DNA knock in (dashes), TRAC knock out together with phosphorothioate and 2′ O-Methyl modified ssDNA knock in (dashes and dots). The plot provides the percent cytotoxicity toward GFP-labeled RPMI8226 CD38-expressing cells after correcting for the cytotoxicity observed toward RPE-labeled K562 cells that do not express CD38.

FIG. 6A to 6C provide graphs of the results of cytokine secretion assays using anti-CD38 CART cells and controls co-cultured with K52 or RPM18226 cells. The T cell cultures tested are as provided in FIG. 5.

FIGS. 7A to 7D provide the results of testing donor DNAs having homology arms (HAs) of different lengths. Cultures were assessed by flow cytometry for loss of TCR (CD3) expression (Y axis) and anti-CD38 expression (X axis).

FIG. SA to SC provide the results of testing double stranded donor DNAs modified by the addition of three PS bonds and three 2′O methyl nucleotides proximal to the 5′ end of one strand of the donor DNA molecule. Cultures were assessed by flow cytometry for loss of TCR expression (Y axis) and anti-CD38 expression (X axis).

FIG. 9A to 9B provide the results of flow cytometry on cells transfected with a ds PS and 2′-OMe-modified donor DNA that included a cassette for expressing an anti-CD19 CAR. The donor was directed to the TRAC exon 1 locus by cotransfection with an RNP. TCR expression is determined on the Y axis and anti-CD19 CAR expression on the Y axis.

FIGS. 10A to 10B provide the results of flow cytometry on cells transfected with a ds PS and 2′-OMe-modified donor DNA that included a cassette for expressing an anti-BCMA CAR. The donor was directed to the TRAC exon 1 locus by cotransfection with an RNP. TCR expression is determined on the Y axis and anti-BCMA CAR expression on the Y axis.

FIGS. 11A to 11C provide the results of flow cytometry on cells transfected with a ds PS and 2′-OMe-modified donor DNA that included a cassette for expressing an anti-CD38 CAR. The donor was directed to the TRAC exon 3 locus by cotransfection with an RNP. TCR expression is determined on the Y axis and anti-CD38 CAR expression on the Y axis.

FIGS. 12A to 12D provide the results of flow cytometry on cells transfected with a ds PS and 2′-OMe-modified donor DNA that included a cassette for expressing an anti-CD19 CAR. In one culture, the donor had homology arms derived from TRAC exon 3 was directed to the TRAC exon 3 locus by cotransfection with an RNP having an exon 3 guide RNA (FIG. 12B). In another culture, the donor had homology arms derived from TRAC exon 1 was directed to the TRAC exon 1 locus by cotransfection with an RNP having an exon 1 guide RNA (FIG. 12C). TCR expression is determined on the Y axis and anti-CD19 CAR expression on the Y axis.

FIGS. 13A to 13D provide the results of flow cytometry on cells transfected with a ds PS and 2′-OMe-modified donor DNA that included a cassette for expressing an anti-C38 CAR and homology arms derived from the TRAC gene or the PD-1 gene. In one culture, the donor had homology arms derived from TRAC exon 1 was directed to the TRAC exon 1 locus by cotransfection with an RNP having an exon 1 guide RNA (FIG. 13D). In another culture, the donor had homology arms derived from the PD-1 locus and was directed to the PD-1 gene by cotransfection with an RNP having a PD-1 gene guide RNA (FIG. 13C). TCR expression is determined on the Y axis and anti-CD38 or PD-1 expression on the Y axis.

FIG. 14 provides the results of cytotoxicity assays using T cell cultures that were transfected with doubly modified (PS and 2′-OMe) donor fragment that included and anti-CD38 CAR construct and PD-1 gene-derived homology arms was targeted to the PD-1 gene by an RNP that included a guide RNA having a target sequence from the PD-1 gene.

FIGS. 15A to 15E provide the results of flow cytometry of cells transfected with a donor DNA comprising an anti-CD38 DAR construct along with an RNP comprising the Cas9 protein (FIG. 15D) or a Cas12a RNP (FIG. 15E). T cell receptor expression is depicted on the Y axis and expression of the anti-CD38 DAR construct on the Y axis. FIG. 15B and FIG. 15C provide the results of transfecting T cells with an RNP that included the Cas9 protein and Cas12a protein, respectively, in the absence of a donor fragment.

FIG. 16 provides a graph of the results of cytotoxicity assays using T cells transfected with an anti-CD38 DAR construct and either a Cas9 RNP or a Cas12a RNP.

FIG. 18A to 18B provide the results of flow cytometry of nonmodified activated T cells (ATC) (FIG. 18A), or T cells transfected with a Cas12a RNP targeting the Tim3 gene along with a donor DNA that included an anti-CD38 DAR construct (FIG. 18B).

FIG. 17 is a table providing the genome location and rate of off-target mutations generated during insertion of the anti-CD38 CAR into the TRAC locus with a Cas9 RNP.

FIGS. 19A to 19H are flow cytometry data of T cells transfected with a Cas9 RNP targeting the TRAC locus and an anti-CD38 DAR donor DNA as well as T cells transfected with a Cas9 RNP targeting the GM-CSF gene in addition to a Cas9 RNP targeting the TRAC locus and an anti-CD38 DAR donor DNA for insertion into the TRAC locus. Also shown, in FIGS. 19E and 19H, is flow cytometry data of T cells transfected with a Cas12a RNP targeting the GM-CSF gene in addition to a Cas12a RNP targeting the TRAC locus and an anti-CD38 DAR donor DNA for insertion into the TRAC locus.

FIG. 20A provides flow cytometry data of T cells transfected with a Cas12a RNP targeting the TRAC locus but no donor DNA and FIG. 20B provides flow cytometry data of T cells transfected with a Cas12a RNP targeting the TRAC locus and an anti-CD20 DAR construct donor DNA.

FIG. 21 is a graph of the percent cytotoxicity of T cells transfected with the anti-CD20 DAR construct and the anti-CEA DAR construct as a function of effector: target cell ratio. The target cells in the assay were Daudi cells.

FIGS. 22A and 22B are bar charts showing the amount of FIG. 22A interferon gamma and FIG. 22B GMCSF secreted by T cells transfected with the anti-CD20 DAR construct and an anti-CD19 CAR construct after antigen stimulation. T cells were stimulated with K562 cells or Daudi cells. Only Daudi cell stimulation resulted in a significant response as indicated by the bars. No cytokine release was detected from unstimulated cells.

FIGS. 23A to 23D provide the results of flow cytometry that assessed the expression of the T cell receptor (CD3) and the anti-CEA CAR construct by engineered T cells: FIG. 23A) T cells transfected with an RNP targeting the TRAC locus but no donor fragment assessed for CD3 (TCR) expression and anti-CEA CAR expression; FIG. 23B) T cells transfected with an RNP targeting the TRAC locus and a donor fragment that included the anti-CEA CAR construct assessed for CD3 (TCR) expression and anti-CEA CAR expression; FIG. 23C) T cells transfected with an RNP targeting the TRAC locus but no donor fragment assessed for CD7 expression and anti-CEA CAR expression; and FIG. 23D) T cells transfected with an RNP targeting the CD7 locus and a donor fragment that included the anti-CEA CAR construct assessed for CD7 expression and anti-CEA CAR expression.

FIG. 24 provides the results of cytotoxicity assays using T cells transfected with the anti-CEA CAR construct targeted to the CD7 locus, T cells transfected with the anti-CEA CAR construct targeted to the TRAC locus; and T cells knocked out at the TRAC locus. The X axis provides effector to target cell ratios in the assays.

FIG. 25 provides a bar graph of IFNγ secretion by T cells transfected with the anti-CEA CAR construct targeted to the TRAC locus and T cells transfected with the anti-CEA CAR construct targeted to the CD7 locus.

### DETAILED DESCRIPTION

### Definitions

Unless specifically indicated otherwise, all technical and scientific terms used herein have the same meaning as commonly understood by those of ordinary skill in the art. In addition, any method or material similar or equivalent to a method or material described herein can be used in the practice of the present disclosure. All publications cited herein are incorporated by reference in their entireties.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines. In some cases, primary cells are cells that have been isolated and then used immediately. In other cases, primary cells cannot divide indefinitely and thus cannot be cultured for long periods of time in vitro.

The term “genome editing” refers to a type of genetic engineering in which DNA is inserted, replaced, or removed from a target DNA. e.g., the genome of a cell, using one or more nucleases. The nucleases create specific double-strand breaks (DSBs) at desired locations in a genome and harness a cell's endogenous mechanisms to repair the induced break by homology-directed repair (HDR) (e.g., homologous recombination) or by nonhomologous end joining (NHEJ). Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. Nuclease-mediated genome editing of a target DNA sequence can be “induced” or “modulated” (e.g., enhanced) using the modified single guide RNAs (sgRNAs) described herein in combination with Cas nucleases (e.g., Cas9 polypeptides or Cas9 mRNA), to improve the efficiency of precise genome editing via homology-directed repair (HDR).

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nonhomologous end joining” or “NHEJ” refers to a pathway that repairs double-strand DNA breaks in which the break ends are directly ligated without the need for a homologous template.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. The term also encompasses nucleic acids containing known analogs of natural nucleotides that have similar binding properties as the reference nucleic acid and are metabolized in a manner similar to naturally occurring nucleotides. A particular nucleic acid sequence also encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al, Nucleic Acid Res. 19:5081 (1991): Ohtsuka et al, J. Biol. Chem. 260:2605-2608 (1985): and Rossolini et al, Mol. Cell. Probes 8:91-98 (1994)). The term nucleic acid is used interchangeably with gene, cDNA, and mRNA encoded by a gene.

When referring to the lengths of nucleic acid molecules, the terms nucleotides and base is pairs may be used interchangeably.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate.

The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “polypeptide,” “peptide,” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms encompass amino acid chains of any length, including full-length proteins, wherein the amino acid residues are linked by covalent peptide bonds.

The term “variant” refers to a form of an organism, strain, gene, polynucleotide, polypeptide, or characteristic that deviates from what occurs in nature.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

The term “stringent conditions” for hybridization refers to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part 1, Second Chapter “Overview of principles of hybridization and the strategy of nucleic acid probe assay”, Elsevier. N.Y.

The term “hybridization” refers to a reaction in which one or more polynucleotides react to form a complex that is stabilized via hydrogen bonding between the bases of the nucleotide residues. The hydrogen bonding may occur by Watson Crick base pairing. Hoogstein binding, or in any other sequence specific manner. The complex may comprise two strands forming a duplex structure, three or more strands forming a multi stranded complex, a single self-hybridizing strand, or any combination of these.

A “recombinant expression vector” is a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter.

“Operably linked” means two or more genetic elements, such as a polynucleotide coding sequence and a promoter, placed in relative positions that permit the proper biological functioning of the elements, such as the promoter directing transcription of the coding sequence.

The term “non-native” means not endogenous to the cell, that is, the construct does not naturally occur in the cell to which it is non-native.

The term “promoter” refers to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al, Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position.

The terms “culture,” “culturing.” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

The term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., Cas nuclease, modified single guide RNA, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other agents, timing of administration, and the physical delivery system in which it is carried.

The term “pharmaceutically acceptable carrier” refers to a substance that aids the administration of an agent (e.g., Cas nuclease, modified single guide RNA, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

The term “increasing stability,” with respect to components of the CRISPR system, refers to modifications that stabilize the structure of any molecular component of the CRISPR system. The term includes modifications that decrease, inhibit, diminish, or reduce the degradation of any molecular component of the CRISPR system.

The term “increasing specificity,” with respect to components of the CRISPR system, refers to modifications that increase the specific activity (e.g., the on-target activity) of any molecular component of the CRISPR system. The term includes modifications that decrease, inhibit, diminish, or reduce the non-specific activity (e.g., the off-target activity) of any molecular component of the CRISPR system.

The term “decreasing toxicity,” with respect to components of the CRISPR system, refers to modifications that decrease, inhibit, diminish, or reduce the toxic effect of any molecular component of the CRISPR system on a cell, organism, subject, and the like.

The term “enhanced activity,” with respect to components of the CRISPR system and in the context of gene regulation, refers to an increase or improvement in the efficiency and/or the frequency of inducing, modulating, regulating, or controlling genome editing and/or gene expression.

The methods and compositions herein use CRISPR/cas systems for the efficient knock out and simultaneous knock in of genes whose expression is desired. CRISPR/Cas systems are now widely used for inducing targeted genetic alterations (genome modifications). Target recognition by a cas protein such as Cas9 requires a “seed” sequence within the guide RNA (gRNA) and a conserved multinucleotide containing protospacer adjacent motif (PAM) sequence upstream of the gRNA-binding region in the target DNA, e.g., host cell genome. (As used herein, the “target sequence” is the sequence adjacent to and (in the Cas9 CRISPR system) immediately upstream of the PAM in the genome. The target sequence (or substantially the same sequence) is engineered into the guide RNA and is sometimes referred to in the art as the “guide sequence” of a guide RNA. For the purposes of the present disclosure, following Ran et al. (2013) Nature Protocols 8:2281-2308, the “target sequence” (or guide sequence) of the guide RNA hybridizes with the opposite strand of the target sequence in the genome.)

Cas/CRISPR RNA-guided endonuclease systems induce permanent gene disruption that utilizes the RNA-guided Cas9 endonuclease to introduce DNA double stranded breaks which trigger error-prone repair pathways to result in frame shift mutations. Examples of CRISPR/Cas systems used to modify genomes are described, for example, in U.S. Pat. Nos. 8,697,359, 10,000,772, 9,790,490, and U.S. Patent Application Publication No. US 2018/0346927, all of which are incorporated herein by reference in their entireties. Cas9, Cas12a, CasX, or other Cas endonucleases may also be used, including but not limited to, Cas1, Cas1B, Cas2, Cas3, Cas4, Cas5, Cash, Cas7, Cas8, Cas9 (also known as Csn1 and Csx12), Cas10, Cas12a (also known as Cpf1), CasX, CasY, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, T7, Fok1, other nucleases known in the art, homologs thereof, or modified versions thereof.

CRISPR/Cas gene disruption occurs when a gRNA sequence specific for a target gene and a Cas endonuclease are introduced into a cell as a complex or to form a complex that enables the Cas endonuclease to introduce a double strand break at the target locus. In some instances, the CRISPR system comprises one or more expression vectors comprising a nucleic acid sequence encoding the Cas endonuclease and a guide nucleic acid sequence specific for the target gene. The guide nucleic acid sequence is designed to be specific for a gene of interest (by homology to the target sequence in the gene) and targets that gene for a cas endonuclease-induced double strand break. Thus, the guide nucleic acid molecule, which is typically an RNA molecule and may be a modified RNA molecule, includes a guide nucleic acid sequence that is found within a loci of the targeted gene (the target site or target sequence). In some embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40, 45, 50, or more nucleotides in length. In some instances, two guide RNAs are used with a cas protein, a crRNA that includes that guide sequence, and a tracrRNA that complexes with the crRNA and cas protein. In some instances, a single guide RNA can be used, which, for example in the case of Cas9, may be a chimeric guide. Some cas endonucleases, e.g., Cas12a, do not use a tracrRNA, i.e., they naturally use only a single guide RNA (the crRNA).

In various embodiments, the cas protein can be expressed in the cell from an introduced gene or RNA molecule. A cas protein can also be introduced, optionally with one or more guide RNAs, or the cas protein can be introduced as a ribonucleoprotein complex with a single guide RNA or two complexed guide RNAs (e.g., a crRNA and a tracrRNA). A guide RNA in some embodiments is expressed from a gene transfected into the target cell, or one or more guide RNAs may be introduced into a cell as RNA molecules. Genes for two or more different guide RNAs can be introduced into a target cell on the same or different vectors. Two or more guide RNAs can be guide RNAs having different guide sequences (for example, targeting different gene loci). In various embodiments of the methods provided herein, a guide RNA can be a chimeric guide (an sgRNA) or can be a crRNA. In some embodiments, a crRNA and a tracrRNA are introduced into the host cell. In some embodiments, the RNA-guided endonuclease is a Cas9 endonuclease and an sgRNA (chimeric guide RNA), an RNP that includes an sgRNA, or a construct encoding an sgRNA is introduced into the cell. Alternatively, a crRNA and a tracrRNA (or constructs encoding a crRNA and a tracrRNA) can be provided in a cell or in an RNP for Cas9 mediated genome modification. In further embodiments, for example embodiments that use Cas12a as the endonuclease that does not require a tracrRNA, a crRNA or a construct encoding a crRNA can be introduced without a tracrRNA. Guide RNAs for cas endonucleases are discussed extensively in US Patent Application Publication US 2018/066242, incorporated herein by reference in its entirely, as well as in U.S. Pat. Nos. 8,697,359, 10,000,772, 9,790,490, and U.S. Patent Application Publication No. US 2018/0346927, all of which are incorporated herein by reference in their entireties.

In addition to their use in generating mutations that occur via error-prone repair pathways such as non-homologous end-joining (NHEJ), cas proteins such as, for example, Cas9, Cas12a, or CasX can be used to insert DNA sequences of interest into a targeted locus, where, in addition to a cas protein and one or more guide RNAs, or constructs for expressing a cas protein and/or one or more guide RNAs, the target cell is also transfected with a donor DNA molecule for insertion into the locus following the activity of the cas endonuclease via homology-directed repair. In various embodiments, a DNA molecule for insertion into a target site includes a DNA sequence of interest, such as, for example, an expression construct, for example a DAR construct, is flanked by sequences having homology to genome sequences on either side of the target site in the host genome. Such homology arms (HAs) can be, for example, from about 50 bp in length to about 2500 bp in length, or from about 100 bp to about 2000 bp in length, or from about 150 bp to about 1500 bp in length. Donor DNA molecules provided herein for use in the compositions, methods, and cells of the invention can have HAs that are, for example, less than about 250 bp in length, less than about 200 bp, less that about 190 bp, less than about 180 bp, less than about 160 bp, or less than about 150 bp in length, for example, from about 50 bp to about 1500 bp in length, from about 50 bp to about 1000 bp in length, from about 50 bp to about 800 bp in length, from about 50 bp to about 600 bp in length, from about 50 bp to about 350 bp in length, from about 50 bp to about 180 bp in length, or from about 100 bp to about 1000 bp in length, from about 140 bp to about 800 bp in length, from about 140 bp to about 600 bp in length, from about 100 bp to about 350 bp in length, from about 100 bp to about 200 bp in length, from about 140 bp to about 800 bp in length, from about 140 bp to about 600 bp in length, from about 140 bp to about 350 bp in length, or from about 140 bp to about 200 bp in length.

The donor DNA can be, for example, at least 50 nucleotides, at least 100 nucleotides, at least 200 nucleotides, at least 225 nucleotides, at least 250 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 1900 nucleotides, at least 2000 nucleotides, at least 2200 nucleotides, at least 2400 nucleotides, at least 2500 nucleotides, at least 2600 nucleotides, at least 2800 nucleotides, at least 3000 nucleotides, at least 3200 nucleotides, at least 3400 nucleotides, at least 3500 nucleotides, at least 3600 nucleotides, at least 3800 nucleotides, at least 4000 nucleotides, at least 4200 nucleotides, at least 4400 nucleotides, at least 4500 nucleotides, at least 4600 nucleotides, at least 4800 nucleotides, at least 5000 nucleotides, at least 5200 nucleotides, at least 5400 nucleotides, at least 5500 nucleotides, at least 5600 nucleotides, at least 5800 nucleotides, at least 6000 nucleotides, at least 6200 nucleotides, at least 6400 nucleotides, at least 6500 nucleotides, at least 6600 nucleotides, at least 6800 nucleotides, at least 7000 nucleotides, at least 7500 nucleotides, at least 8000 nucleotides, at least 8500 nucleotides, at least 9000 nucleotides, at least 9500 nucleotides, or at least 10,000 nucleotides, or the corresponding number of base pairs (bp) in length where the donor fragment is double-stranded or substantially double stranded.

A donor DNA as provided in the composition, methods, and systems disclosed herein can be single-stranded, double-stranded, or substantially double-stranded. A donor DNA may be single-stranded or double-stranded, which includes a substantially double-stranded molecule, where the substantially double-stranded donor DNA can be double stranded with the exception of short (e.g., 10 or fewer, 8 or fewer, 6 or fewer, or 3 or fewer) stretches of nucleotides that are not base-paired with an opposite strand which may occur at the ends of the fragment or internally, where such short stretches are less than 50%, less than 30%, less than 10%, or less than 5% of the nucleotide length of the fragment.

Donor DNA molecules can be modified at the base moiety, sugar moiety, or phosphodiester backbone. The modifications can be conveniently introduced by PCR amplification of a template that includes the construct to be inserted into the target locus of the genome, typically flanked by homology arms. The PCR amplification of the donor template produces the donor DNA molecule, where the amplification uses primers having the desired modifications that are then incorporated into the donor DNA product.

Nucleic acid modifications can include, but are not limited to: 2′O methyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Nucleic acid modifications can include, for example, deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-O-methyl or 2′-O-allyl sugars. For nucleosides that include a pentofuranosyl sugar, the phosphate group can be linked to the 2′, the 3′, or the 5′ hydroxyl moiety of the sugar.

Nucleotides are nucleosides that further include a phosphate group covalently linked to the sugar portion of the nucleoside to form the “internucleoside backbone” of the nucleic acid molecule. Naturally-occurring RNA and DNA molecules have a 3′ to 5′ phosphodiester linkage throughout the backbone. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195 and Hyrup et al. (1996) Boorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone. A phosphorothioate (PS) bond (i.e., a phosphorothioate linkage) substitutes a sulfur atom for a non-bridging oxygen in the phosphate backbone of a nucleic acid. This modification renders the internucleotide linkage resistant to nuclease degradation.

Modifications include nucleic acids containing modified backbones or non-natural internucleoside linkages. Nucleic acids having modified backbones include those that retain a phosphorus atom in the backbone and those that do not have a phosphorus atom in the backbone. Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Nucleic acids having inverted polarity comprise a single 3′ to 3′ linkage at the 3′-most internucleotide linkage i.e. a single inverted nucleoside residue which may be a basic (the nucleobase is missing or has a hydroxyl group in place thereof).

In some embodiments, a donor DNA includes one or more phosphorothioate and/or heteroatom internucleoside linkages. MMI type internucleoside linkages are disclosed in U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Additional modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Morpholino backbone structures as described in, e.g., U.S. Pat. No. 5,034,506. For example, in some embodiments, a donor DNA can include one or more nucleotides having a 6-membered morpholino ring in place of a deoxyribose ring. In some of these embodiments, a phosphorodiamidate or other non-phosphodiester internucleoside linkage replaces a phosphodiester linkage.

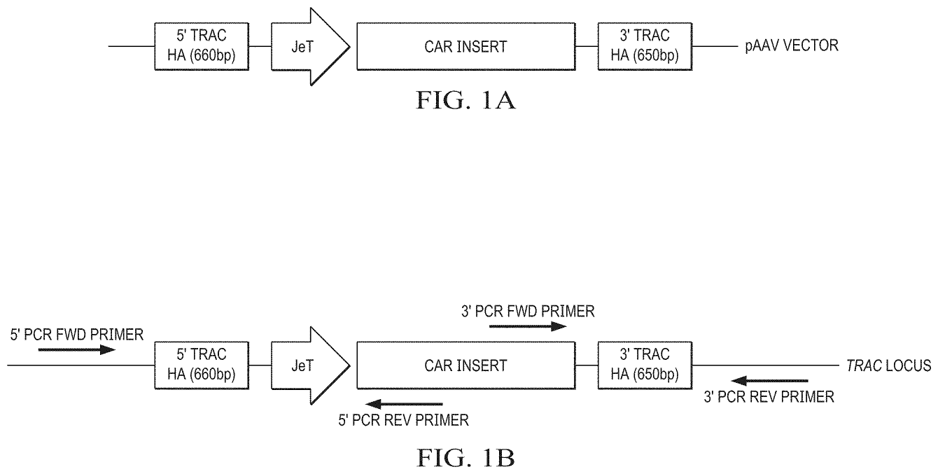

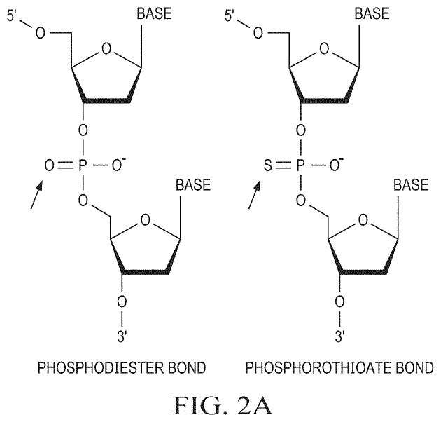

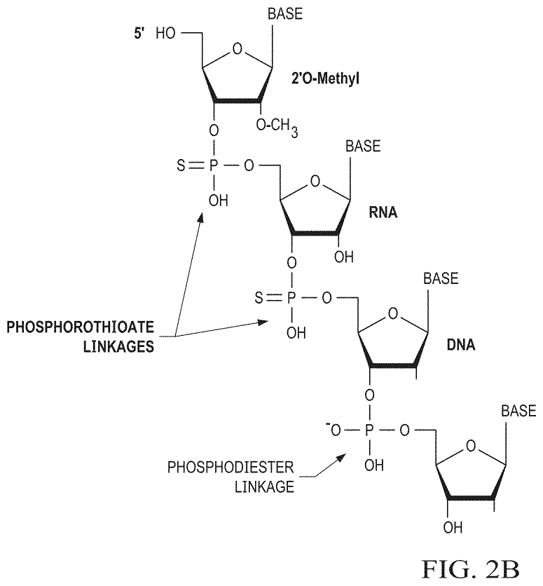

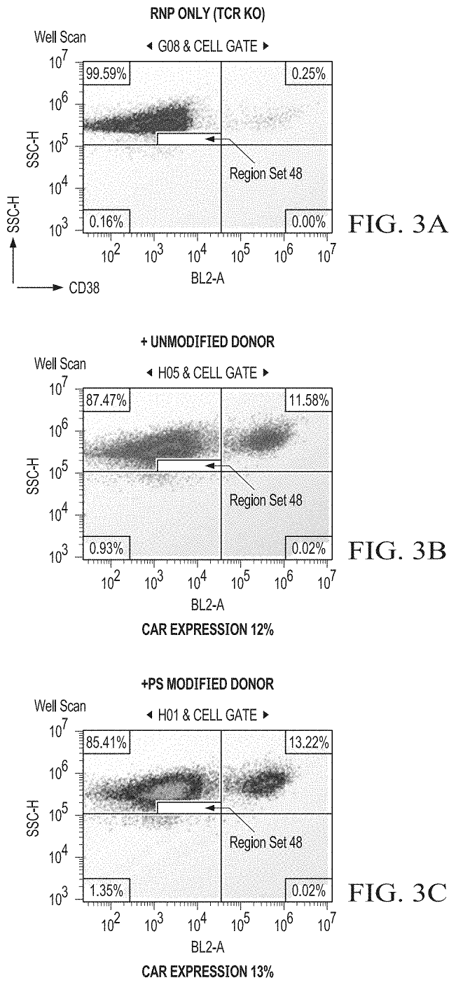

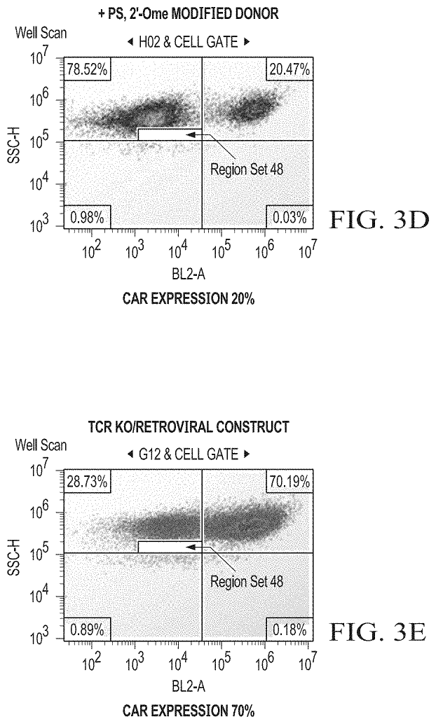

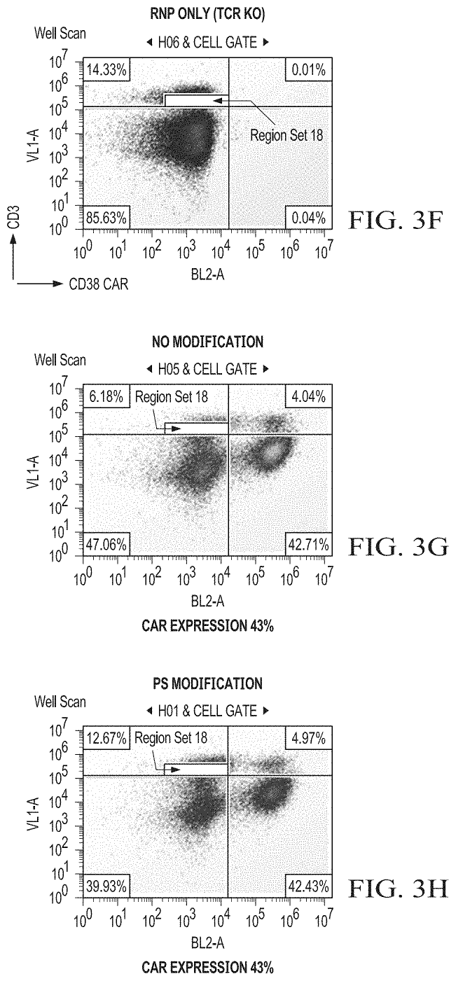

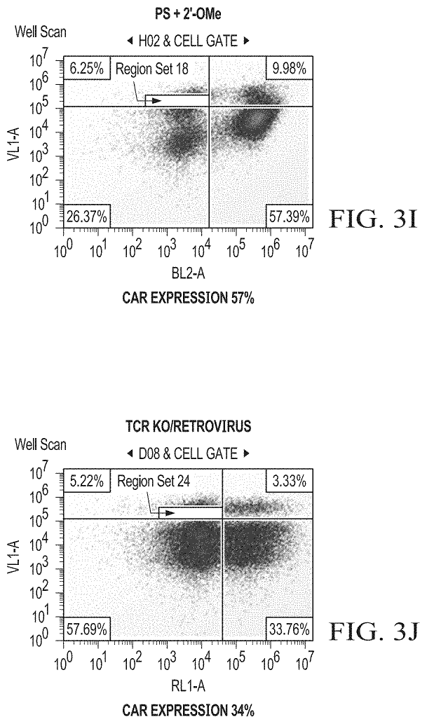

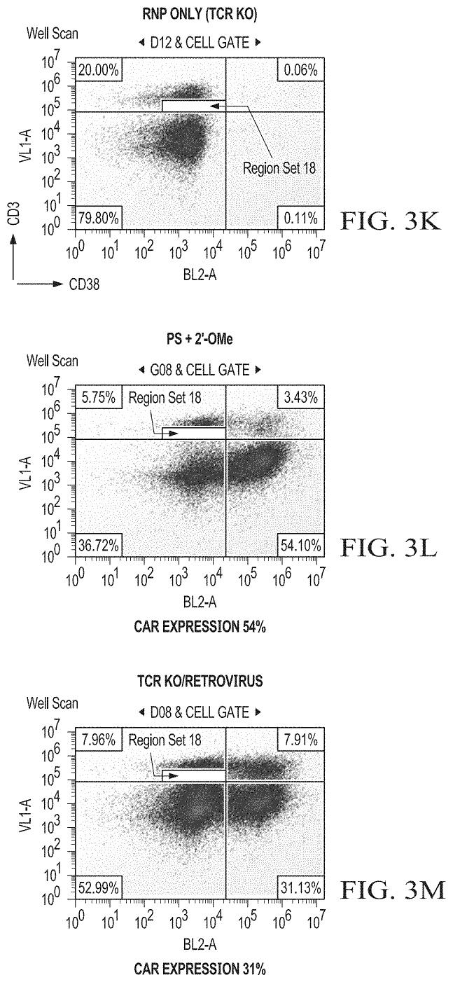

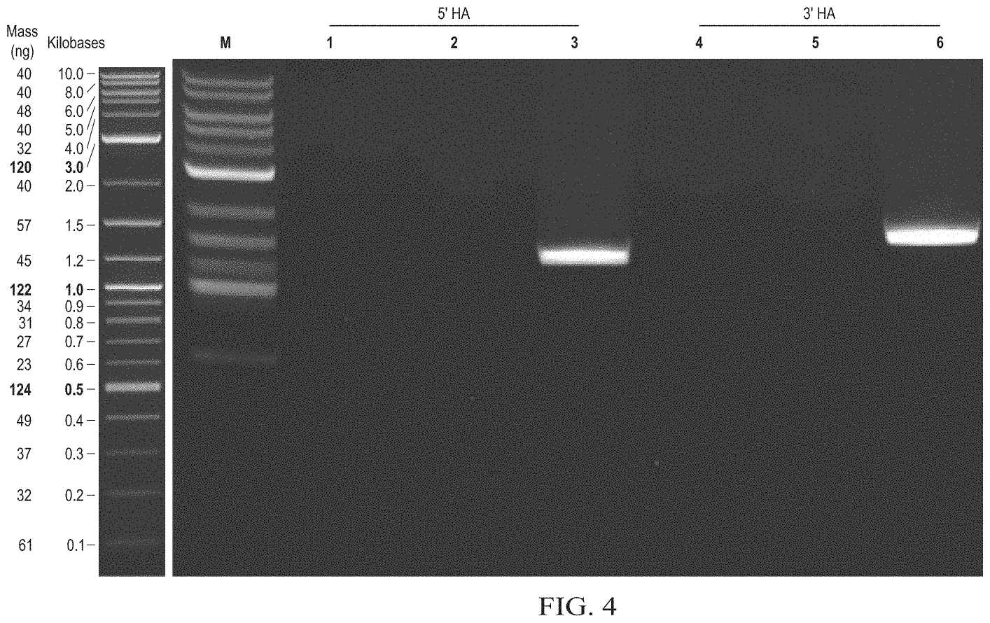

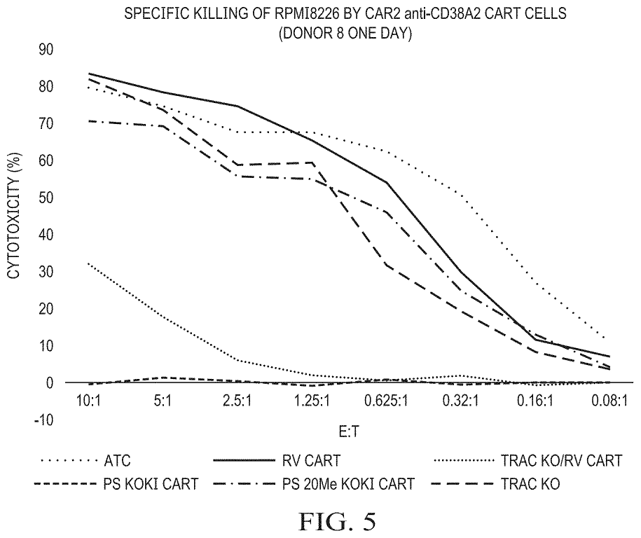

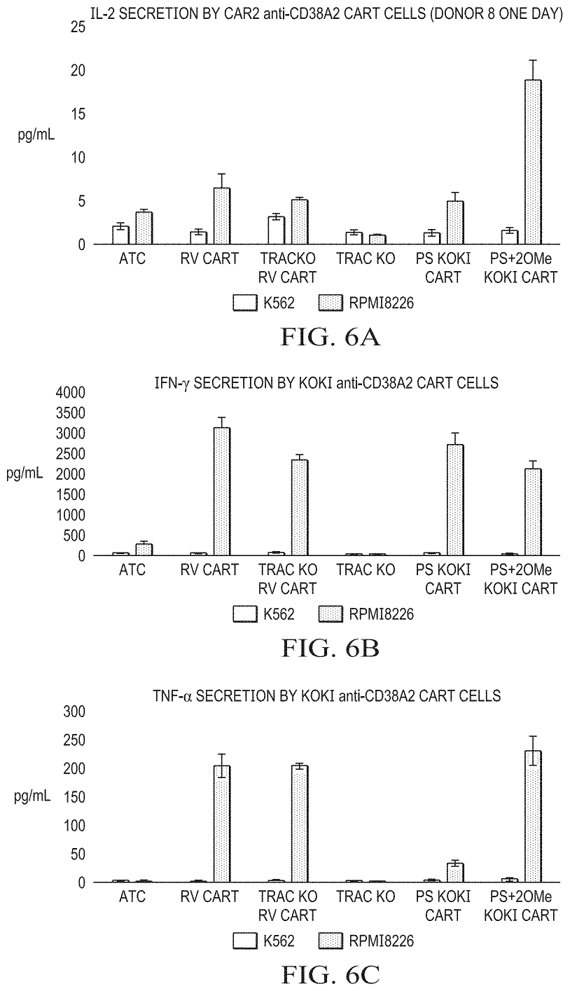

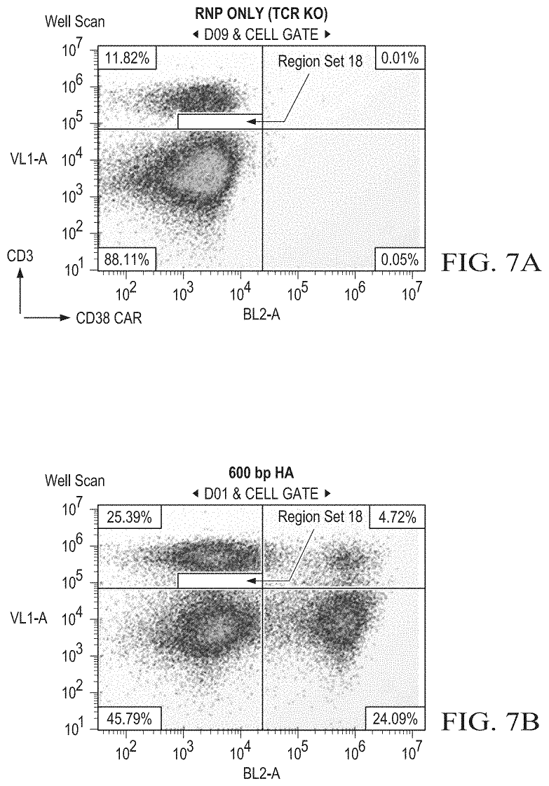

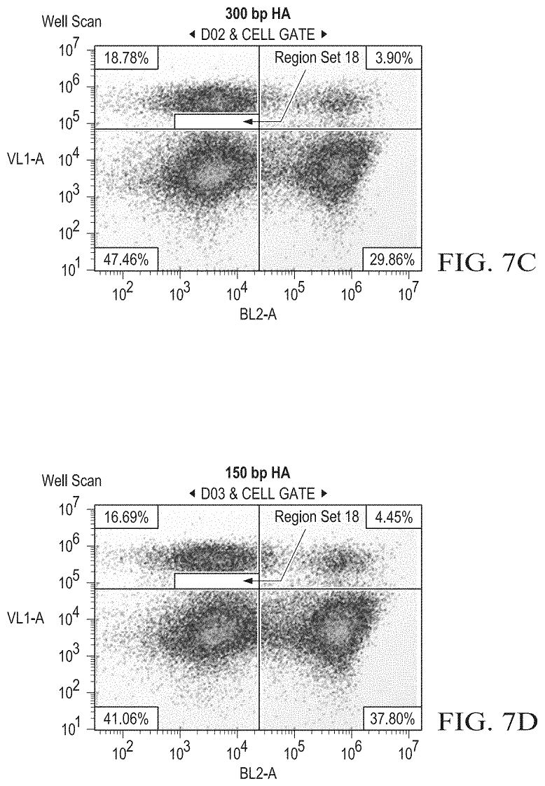

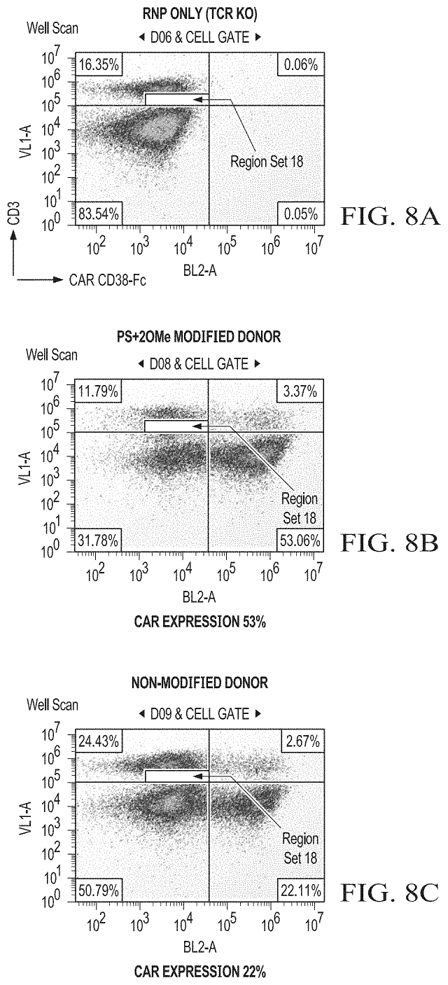

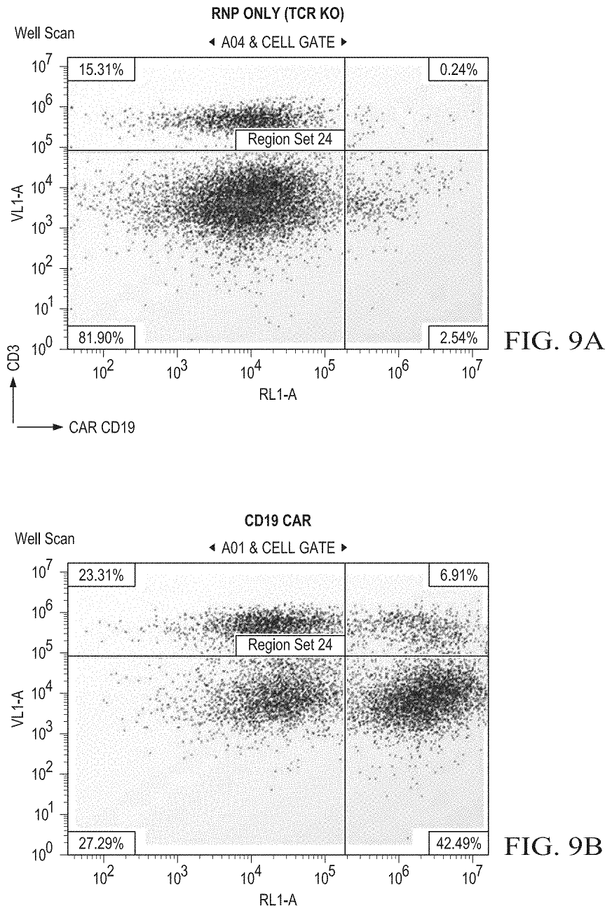

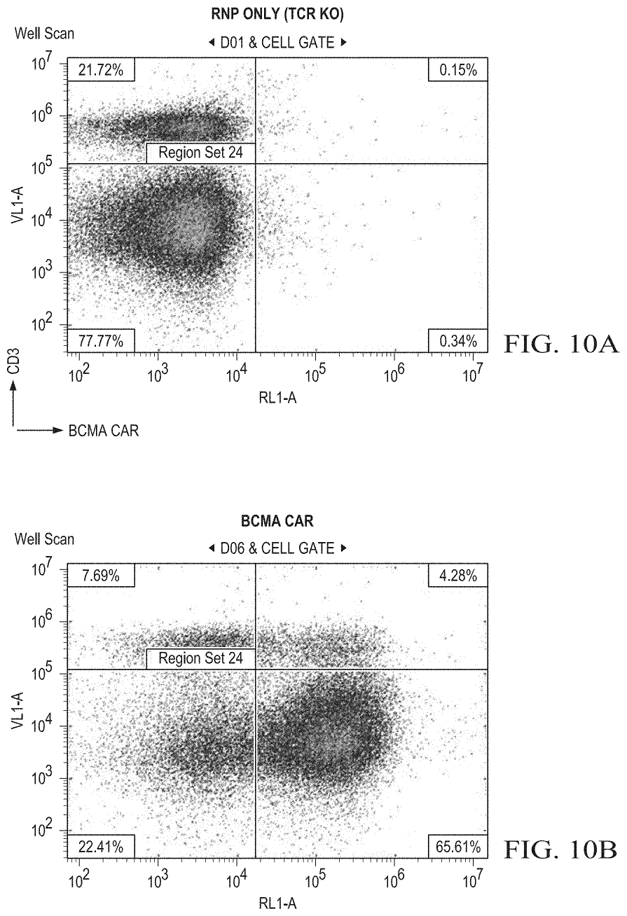

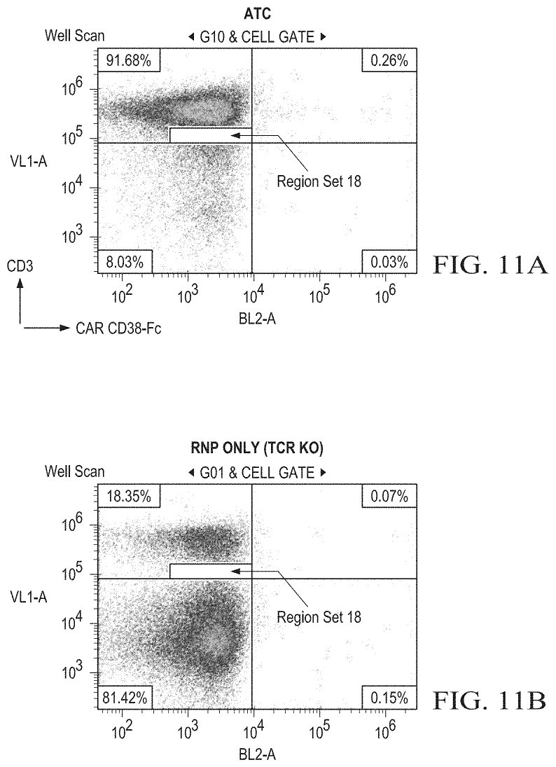

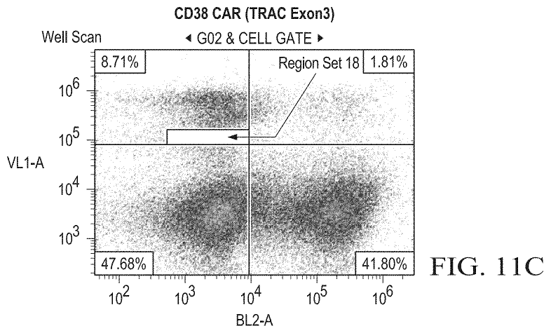

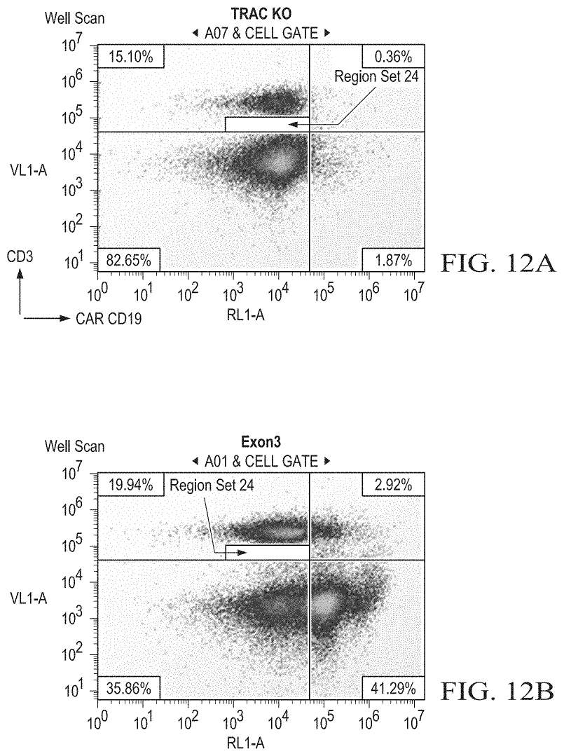

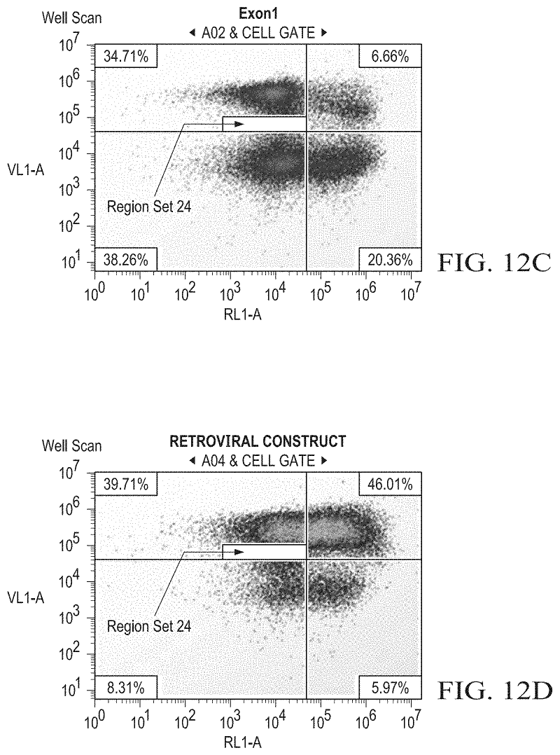

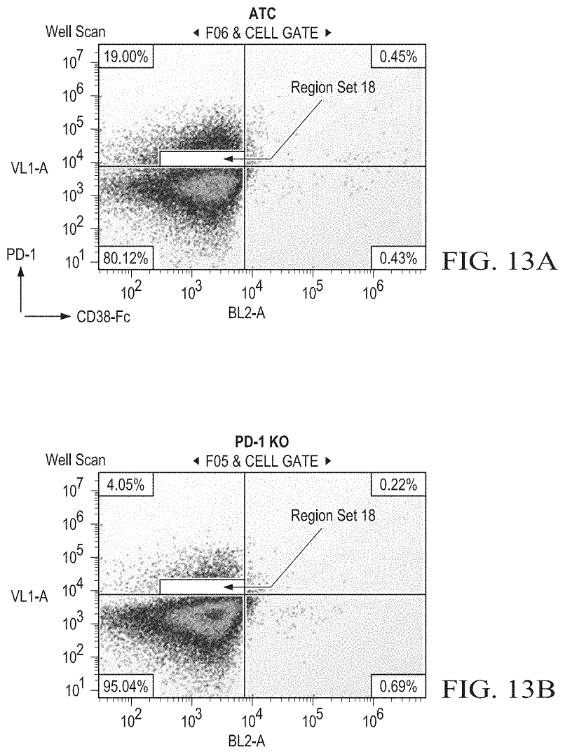

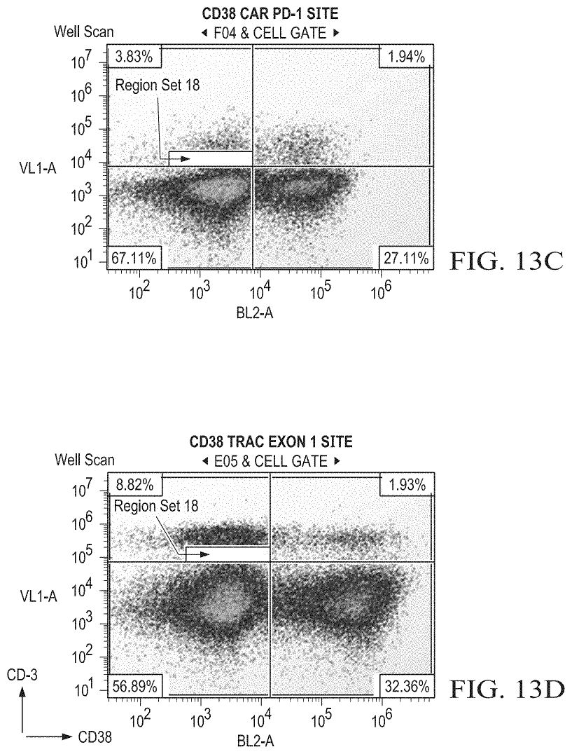

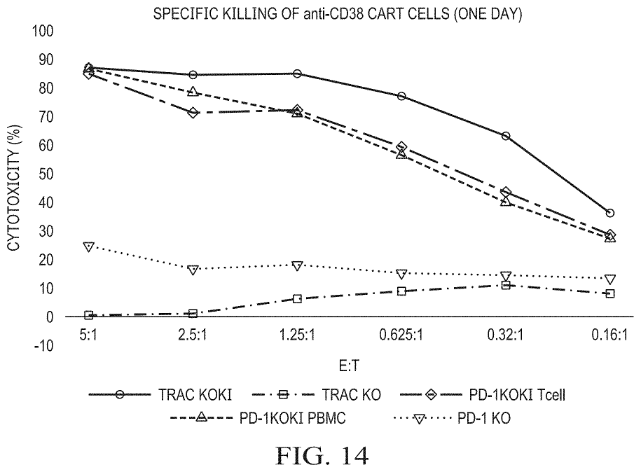

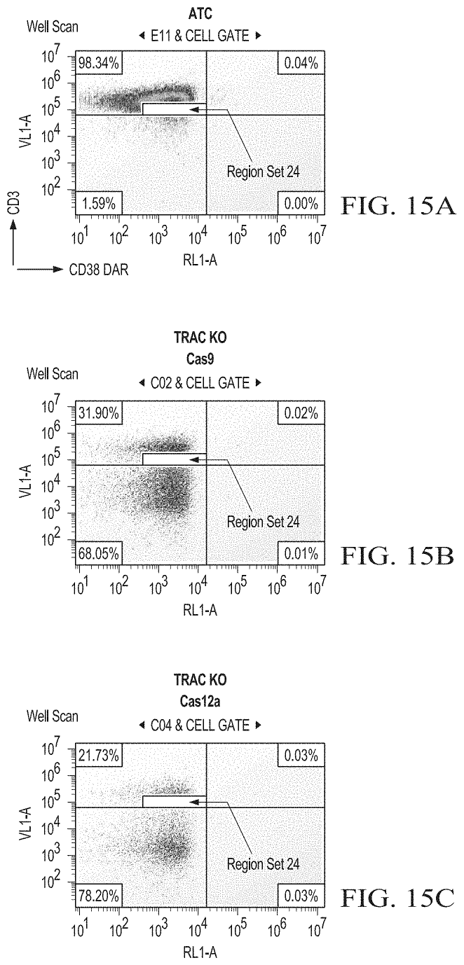

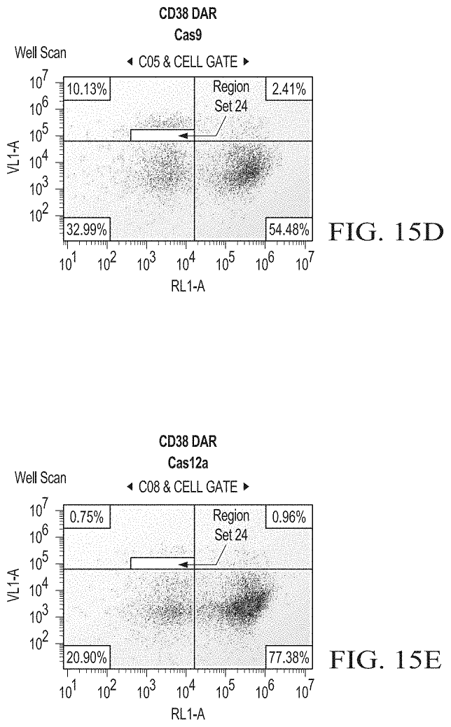

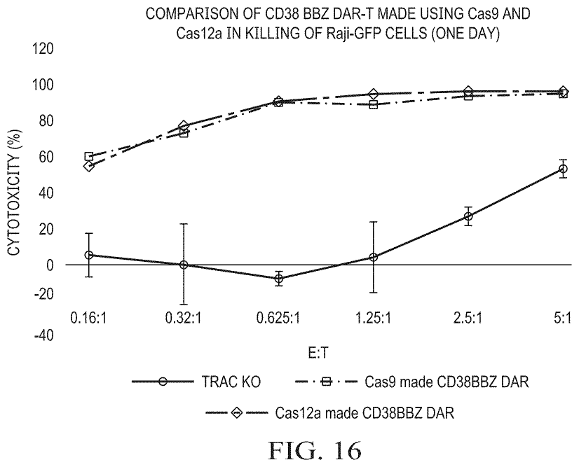

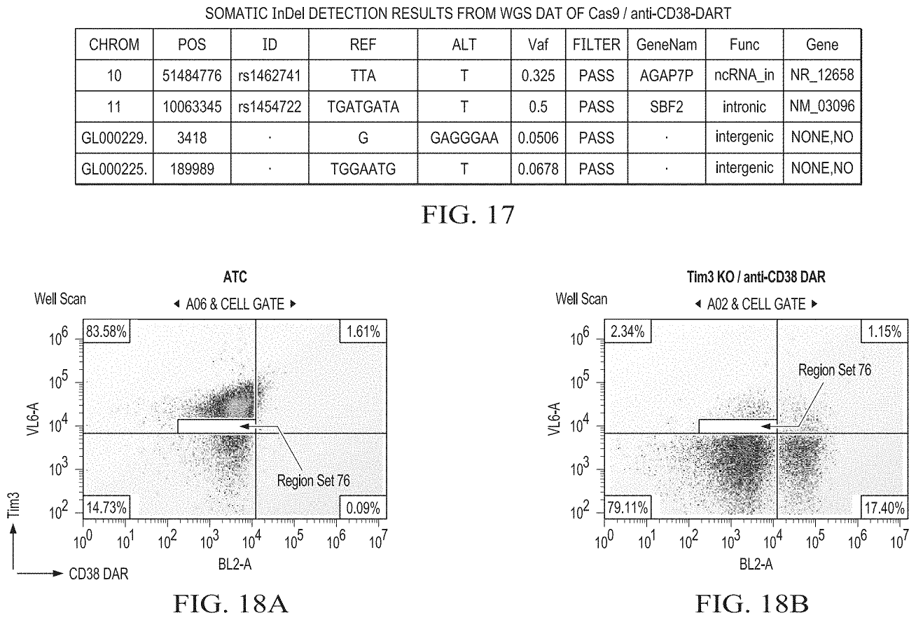

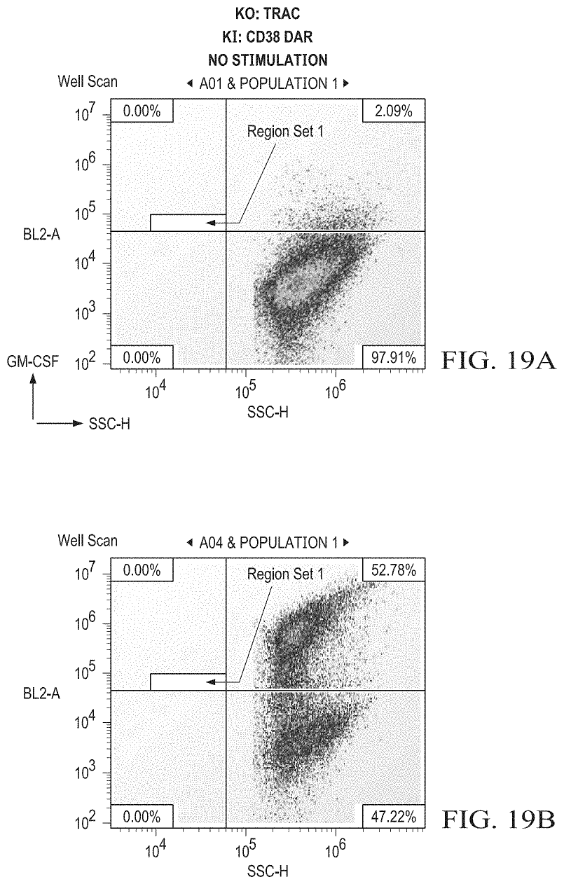

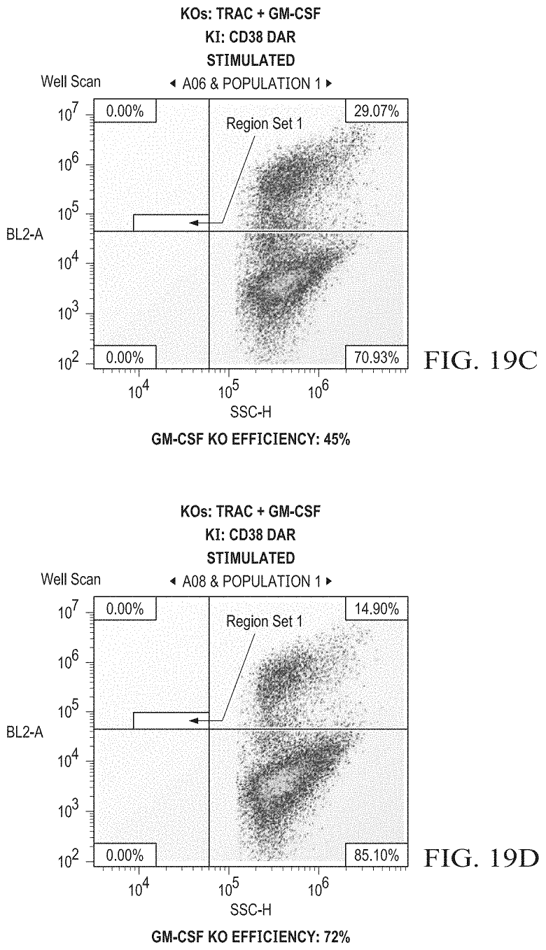

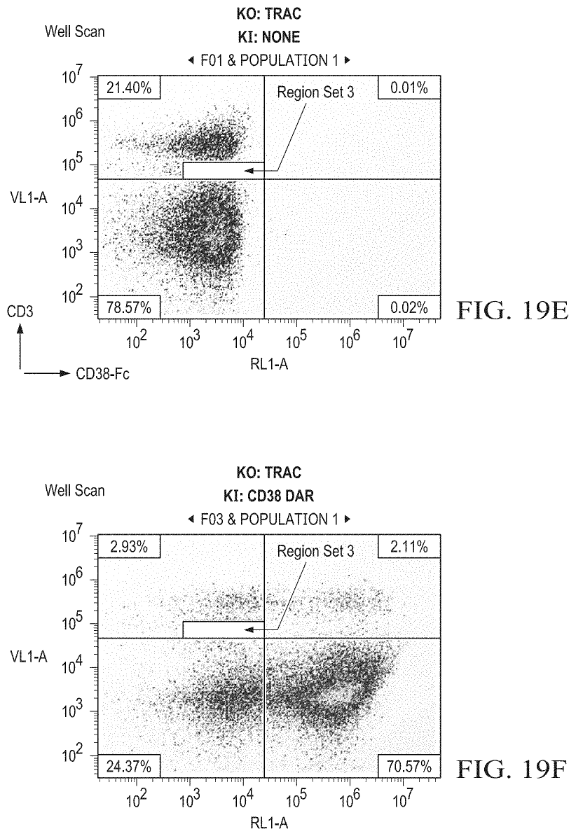

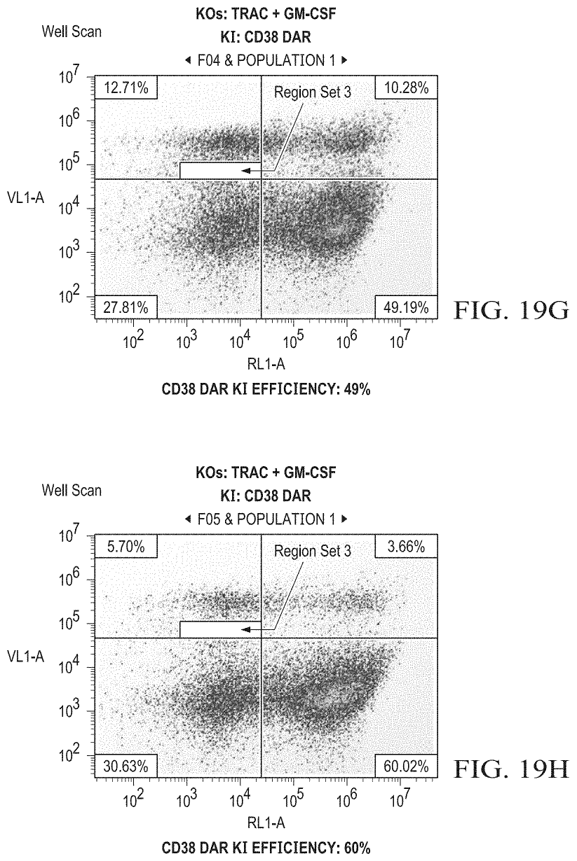

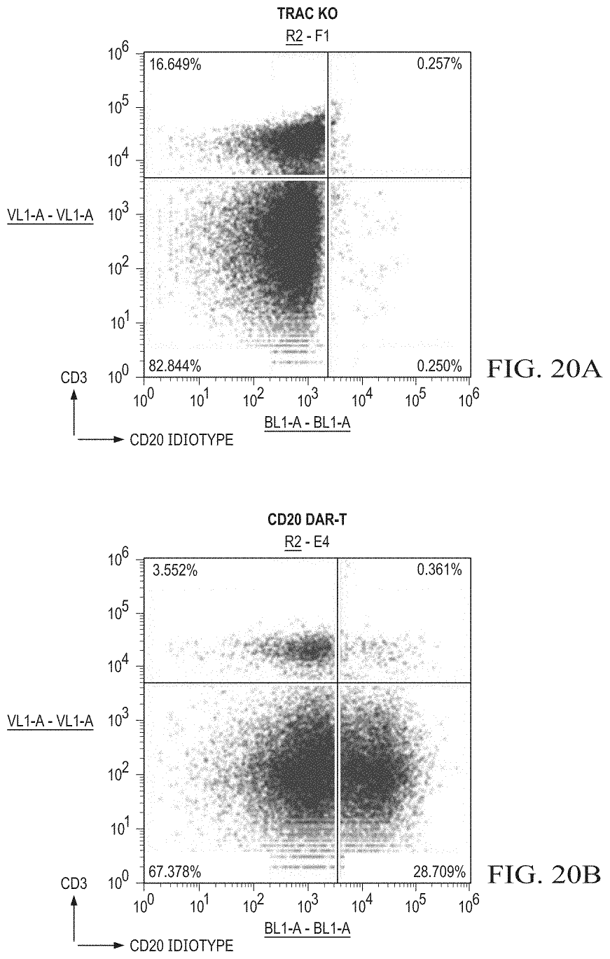

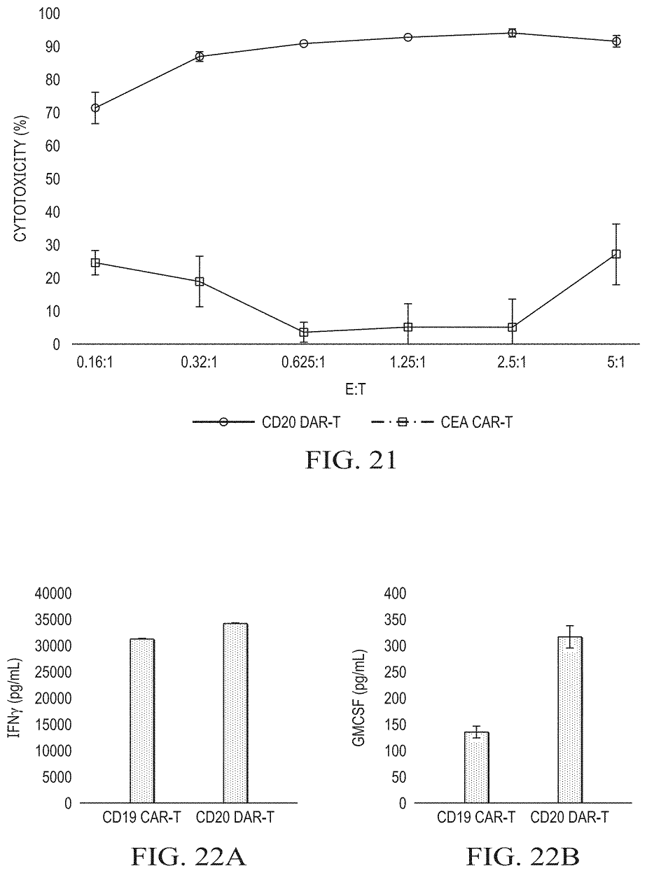

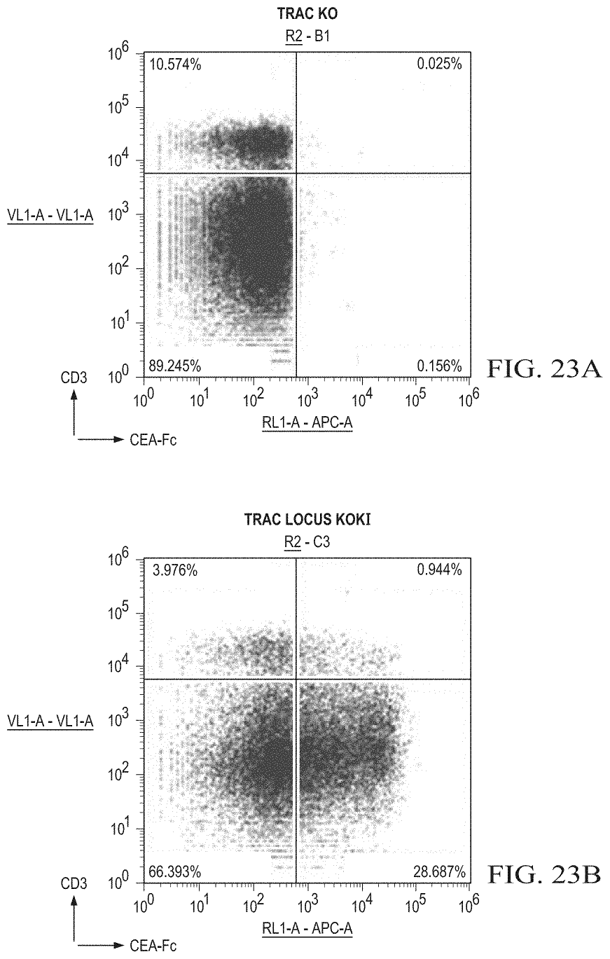

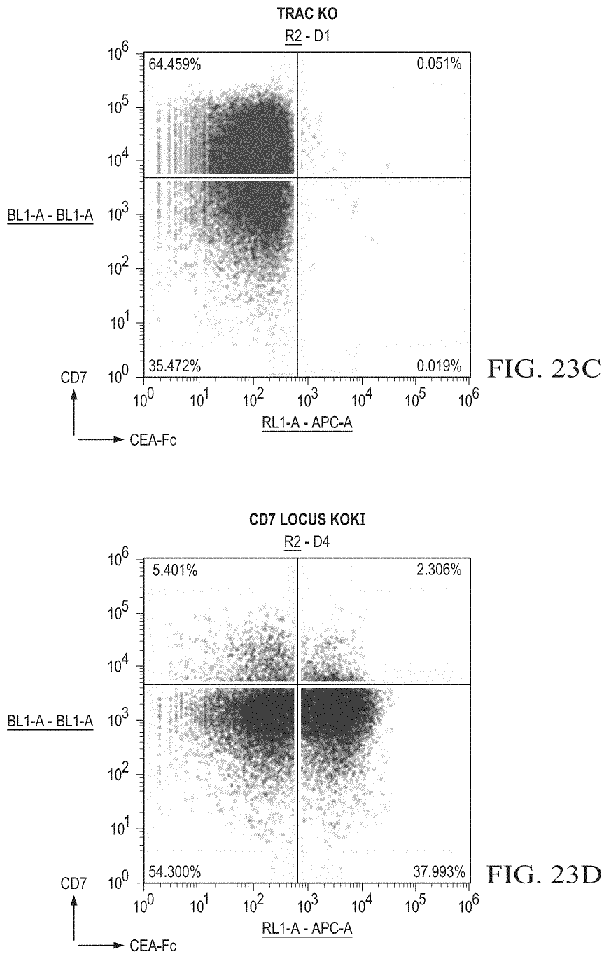

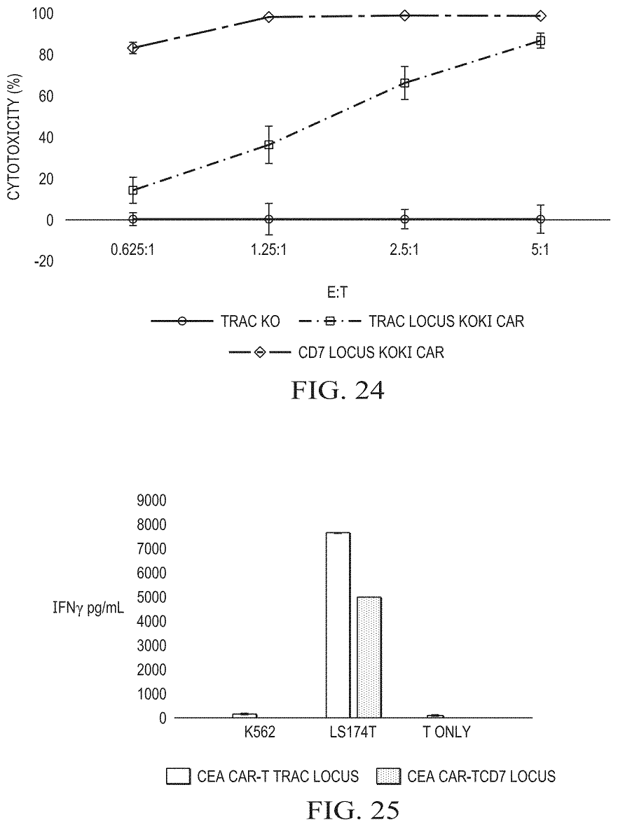

2-O-Methyl modified nucleotides (see FIG. 2B) are naturally occurring modifications of RNA found in tRNA and other small RNAs that arises as a post-transcriptional modification. Oligonucleotides can be directly synthesized that contain 2′-O-Methyl nucleotides. 2′ Fluoro modified nucleotides (e.g., 2′ Fluoro bases) have a fluorine modified sugar which increases binding affinity (Tm) and may also confer some relative nuclease resistance.

In some embodiments, a donor DNA molecule has one or more nucleotides that are 2′-O-Methyl modified nucleotides. In some embodiments, a donor DNA molecule has one or more nucleotides that are 2′ Fluoro modified nucleotides. In some embodiments, a donor DNA molecule one or more LNA, PNA, pPNA, or pHypPNA nucleotides. In some embodiments, a donor DNA has one or more nucleotides that are linked by a phosphorothioate bond (i.e., the donor DNA has one or more phosphorothioate linkages. In some embodiments, a donor DNA molecule has a combination of modified nucleotides. For example, a donor DNA can have one, two, three, or more phosphorothioate linkages in addition to having one or more nucleotides with other modifications (e.g., a 2′-O-Methyl nucleotide and/or a 2′ Fluoro modified nucleotide and/or a LNA base). These modifications preferably occur only on one strand of a double-stranded DNA molecule, and most advantageously at the 5′ end of one strand of the double-stranded DNA molecule, for example, within 20, within 10, or within 5 nucleotides of the 5′ terminus of the double-stranded DNA molecule.

Introduction of the donor DNA can be by any means of introducing DNA into the host cell, such as, for example, electroporation, nucleofection, or lipofection. In exemplary embodiments, the donor DNA is not introduced via viral transduction. For example, the donor can be provided as a synthesized DNA molecule that is electroporated or by other means transfected into the cell along with one or more RNPs that include a cas protein and guide RNA targeting the selected insertion locus. The targeted insertion locus can optionally be a gene whose disruption (“knockout”) is desired, such that insertion of the expression construct simultaneously ablates expression of the gene. The donor DNA can include sequences homologous to the host genome at the target site to facilitate HDR following cleavage of the target site by the cas nuclease. Alternatively a donor DNA can be introduced into a cell before or after a cas nuclease and/or guide RNA, or a construct for expressing a cas nuclease and/or guide RNA is introduced into the cell.

Methods are provided herein that provide a high efficiency targeted gene integration approach. The methods can be used for genome engineering of any cell type, and can be used, for example, in applications where engineered cells are introduced into a patient.

Methods are provided herein that provide a high efficiency targeted gene integration at a first site, with disruption of the endogenous gene at the first site, along with simultaneous knock out of a gene at a second site. For example, a CAR or DAR construct may be inserted into the TRAC or TRBC locus, thereby inactivating the TRAC or TRBC gene, while simultaneously knocking out a checkpoint inhibitor or immune modulator gene, such as, as nonlimiting examples, a gene encoding GM-CSF, PD-1, TIM3, CTLA-4, PDCD1, LAG3, etc. Methods of knockout/knockin at a first locus with simultaneous knockout of a second locus include: introducing into a cell: a first RNP comprising a first RNA-guided nuclease complexed with a first guide RNA targeting a first gene locus, a second RNP comprising a second RNA-guided nuclease complexed with a second guide RNA targeting a second gene locus, and a donor DNA, modified as disclosed herein and having homology arms with homology to genome sequences at the first gene locus. The first and second RNA-guided endonucleases can be the same or different. For example, in some embodiments the first and second RNA-guided endonucleases are both cas9 nucleases. In other examples, the first and second RNA-guided endonucleases are both cas12a nucleases. In further examples, the first RNA-guided endonuclease is cas9 and second RNA-guided endonuclease is cas12a. In further examples, the first RNA-guided endonuclease is cas12a and second RNA-guided endonuclease is cas9. The methods result in modification of the cells where the donor DNA is inserted into the first locus and the gene at the second locus is disrupted.

In some embodiments, the methods provided herein can be used for installing a cancer treating construct, e.g. a CAR, for example against any of CD38, CD19, CD20, CD123, BCMA and the like into T cells. The efficiency of gene transfer can reach 40-80%. This approach, employing a targeted gene integration, can be used for both autologous and allogenic approaches, and importantly, does not carry a risk of secondary and unwanted cell transformation when engineered cells are introduced into a patient and is therefore safer than current conventional approaches. Additional advantages include a modified guide strand, reliable gene integration, integration of large genes, gene integration of a CAR, and gene integration of a CAR with high expression.

The examples disclose making CAR-T cells via RNA-guided endonuclease-mediated genome editing that uses phosphorothioate and 2′ O-methyl modified single-stranded or double-stranded donor DNA synthesized by PCR. Preferably, the modified single-stranded (ss) or double-stranded (ds) DNA is produced by adding three PS bonds to the nucleotides within 10 nucleotides or five nucleotides of the 5′-end of one primer. Without limiting the invention to any particular mechanism, it is believed the PS modification inhibits exonuclease degradation of the modified strand of the donor DNA. Nucleotides within ten or within five nucleotides of the 5′ end of the primer were also modified with 2′ O-methyl to avoid the non-specific binding which is caused by phosphorothioate bonds. The phosphorothioate and 2′ O-methyl modified ds donor DNA and ss donor DNA can be made through PCR, asymmetric PCR or reverse transcription. In the alternative, the final ds DNA product of a synthesis can be modified with phosphorothioate and 2′ O-methyl and dsDNA can be produced with modification on one strand only.

There is further disclosed a donor DNA construct, such as a donor DNA construct having chemical modifications such as phosphorothioate and 2′ O-methyl that include a CAR construct, i.e., are designed for inserting a CAR (chimeric antigen receptor) into a defined genomic site of a host cell. Further, the present disclosure provides a host cell transfected with a CAR that lacks viral vectors that can present a safety concern.

This process—using a donor DNA with modifications on one strand—can increase knock-in efficiency at least two-fold, which is comparable with viral vector methods and has advantages for site specificity of integration and very stable for CAR expression in T cells compared to conventional retrovirus or lentivirus approaches. At least double modification of one donor chain with phosphorothioate and/or 2′ O-methyl can increase knock-in efficiency. This one step knock-out/knock-in method provides a faster and cheaper CAR-T production process for multiple cancer therapy. The ability to use double stranded DNA and avoid nuclease treatment of the donor construct and recovery of the single strand which is laborious and reduces yields is another benefit of the method.

In this application, we present a simple and robust method for knock in long dsDNA or ssDNA (e.g. ˜3 kb Anti-CD38 CAR and CD19 CAR) by modified dsDNA or ssDNA donor with phosphorothioate and 2′ O-methyl modification. We show that modified long dsDNA and ssDNA sequences are highly efficient HDR templates for the integration of CAR into primary T cells. Further we demonstrate that this method has advantages for site specificity of integration and very stable for CAR expression in T cells compared to conventional retrovirus or lenti-virus approaches.

The present disclosure provides methods for expressing a CAR gene in a primary cell, the method comprising introducing into the primary cell:

(a) a single guide RNA (sgRNA) comprising a first nucleotide sequence that is complementary to the selected knockout nucleic acid and a second nucleotide sequence that interacts with a CRISPR-associated protein (Cas) polypeptide, wherein one or more of the nucleotides of the sgRNA sequence are optionally modified nucleotides: and
(b) a Cas polypeptide, an mRNA encoding a Cas polypeptide, and/or a recombinant expression vector comprising a nucleotide sequence encoding a Cas polypeptide, or Cas polypeptide wherein the modified sgRNA guides the Cas polypeptide to the site of knockout nucleic acid, and (c) a donor target DNA comprising a 5′ HA sequences, a promoter sequence, a CAR construct, and 3′HA sequence, wherein the donor target DNA is preferably double-stranded and has both or preferably one strand modified with at least one phosphothioate bond within five nucleotides of the 5′-end of the donor for reducing 5′exonuclease cleavage, and optionally includes one, two three, or four 2′-O-methyl-modified nucleotides within 5 nucleotides of the 5′ end. Preferably the opposite strand to the modified strand has a 5′ terminal phosphate. The promoter is operable in the primary cell, which can be, for example, a T cell.

The present disclosure provides a method for inducing gene expression of a CAR gene in a primary cell, the method comprising introducing into the primary cell:

(a) a crRNA comprising a nucleotide sequence that is complementary to the selected target nucleic acid, wherein one or more of the nucleotides in the guide RNA are optionally modified nucleotides and a tracrRNA: and
(b) a Cas polypeptide, an mRNA encoding a Cas polypeptide, and/or a recombinant expression vector comprising a nucleotide sequence encoding a Cas polypeptide, or a Cas polypeptide; wherein the crRNA guides the Cas polypeptide to the site of knockout nucleic acid: and (c) a donor target DNA comprising a 5′ HA sequences, a promoter sequence, a CAR construct, and 3′HA sequence, wherein the donor target DNA is preferably double-stranded and has both or preferably one strand modified with at least one phosphorothioate bond within five nucleotides of the 5′-end of the donor for reducing 5′exonuclease cleavage, and optionally includes one, two three, or four 2′-O-methyl-modified nucleotides within 5 nucleotides of the 5′ end. Preferably the opposite strand to the modified strand has a 5′ terminal phosphate. The promoter is operable in the primary cell, which can optionally be a T cell.

In some embodiments, the cells are modified for cell-based therapies. The cells can be, as nonlimiting examples, stem cells, fibroblasts, glial cells, myocytes, or hematopoietic cells and can be modified using methods as disclosed herein and transferred into a patient. The cells can be autologous or allogeneic with respect to the patient. If allogeneic, the cells can be from one or multiple donors.

### EXAMPLES

The examples show the advantages of the disclosed process to provide high transfection efficiency without the use of viral vectors for knocking in donor DNA and knocking out a targeted endogenous gene such as a T cell receptor (TCR) or PD-1 gene. Also exemplified is simultaneous gene knock-out and gene knock-in at a first locus and gene knock-out at a second locus resulting from a single transfection.

Buffy coats from healthy volunteer donors were obtained from the San Diego blood bank. Some fresh whole blood or leukapheresis products were obtained from StemCell Techologies (Vancouver, Canada). Peripheral blood mononuclear cells (PBMCs) were isolated by density gradient centrifugation. PBMCs were activated with CD3 antibody (BioLegend, San Diego, Calif.) 100 ng/mL for two days in AIM-V medium (ThermoFisher Scientific, Waltham, Mass.) supplemented with 5% fetal bovine serum (Sigma, St. Louis, Mo.) with 300 U/mL IL-2 (Proleukin) at a density of 10⁶ cells per mL. The medium was changed every two to three days, and cells were re-plated at 10⁶ per mL. This treatment selectively amplifies T cells in the culture. In some experiments, cells were cultured in CTS™ OpTmizer™ T Cell Expansion SFM (ThermoFisher Scientific) supplemented with 5% CTS™ Immune Cell SR (Thermofisher Scientific) with 300 U/mL IL-2 (Proleukin) at a density of 10⁶ cells per mL. In some experiments T cells were isolated from PBMCs using magnetic negative selection using EasySep™ Human T Cell Isolation Kit or CD3 positive selective kit (Stemcell Technologies) or Dynabeads™ Human T-Expander CD3/CD28 (ThermoFisher Scientific) according to the manufacturer's instructions.

For use in cytotoxicity assays, RPMI-8226 (multiple myeloma cell line) cells, which express CD38, were transduced to express green fluorescent protein (GFP) and K562 (human immortalized myelogenous leukemia) cells, which do not express CD38, were transduced to express R-phycoerythrin (RPE). Both cell lines were cultured in RPMI1640 medium (ATCC) supplemented with 10% fetal bovine serum (Sigma). CAR plasmids were generated with an In-Fusion® HD Cloning Kit (Takara Bio USA, Inc, Mountain View, Calif.). Backbone plasmid pAAV-MCS (Cell Biolabs (San Diego, Calif.)) was used for generating the genetic constructs that were used as PCR templates for generating donor fragments.

In some experiments, retrovirus-transduced T cells were compared with cas-mediated knock-in cells. Transduction of T cells with the retroviral construct was performed essentially as described in Ma et al. (2004) The Prostate 61:12-25; and Ma et al. (2014) The Prostate 74(3):286-296 (the disclosures of which are incorporated by reference herein in their entireties). In brief, the anti-CD38 CAR (or other construct) plasmid DNA was transfected into the Phoenix-Eco cell line (ATCC) using FuGene reagent (Promega, Madison, Wis.) to produce ecotropic retrovirus, then harvested transient viral supernatant (ecotropic virus) was used to transduce PG13 packaging cells that express the GaLV envelope protein for the production of retrovirus for infection of human cells. Viral supernatant from PG13 cells was used to transduce activated T cells (or PBMCs) two to three days after CD3 or CD3/CD28 activation. Activated human T cells were prepared by activating normal healthy donor peripheral blood mononuclear cells (PBMC) with 100 ng/ml mouse anti-human CD3 antibody OKT3 (Orth Biotech, Rartian, N.J.) or anti-CD3, anti-CD28 T-cell TransAct Reagent (Miltenly Biotech, San Diego, Calif.) according to the manufacturer's manual and 300-1000 U/ml IL-2 in AIM-V growth medium (GIBCO-Thermo Fisher scientific, Waltham, Mass.) supplemented with 5% FBS for two days. 5×10⁶ activated human T cells were transduced in a 10 sg/ml retronectin (Takara Bio USA) pre-coated 6-well plate with 3 ml viral supernatant and were centrifuged at 1000 g for 1 hour at 32° C. After transduction, the transduced T cells were expanded in AIM-V growth medium supplemented with 5% FBS and 300-1000 U/ml IL2.

TABLE 1
Primers used for generating donor DNAs:
an asterisk indicates a phosphorothioate (PS) linkage;
Am, 2′-O-methylated deoxyadenosine; Cm, 2′-O-methylated
deoxycytosine; Gm, 2′-O-methylated deoxyguanosine

| Primer | Sequence | SEQ ID NO |
|---|---|---|
| Forward primer for generating donor DNA | 5′-T*Gm*Gm*AmGCTAGGGCACCATATT-3′ | 8 |
| having 660 and 650 nt HAs from TRAC | | |
| gene exon 1 | | |
| Reverse primer for generating donor DNA | p-5′-CAACTTGGAGAAGGGGCTT-3′ | 9 |
| having 660 and 650 nt HAs from TRAC | | |
| gene exon 1 | | |
| Forward primer for generating donor DNA | 5′-C*Cm*Am*TGmCCTGCCTTTACTCTG-3′ | 14 |
| having 375 and 321 nt HAs from TRAC | | |
| gene exon 1 | | |
| Reverse primer for generating donor DNA | p-5′-TCCTGAAGCAAGGAAACAGC-3′ | 15 |
| having 375 and 321 nt HAs from TRAC | | |
| gene exon 1 | | |
| Forward primer for generating donor DNA | 5′-A*TCm*Am*CmGAGCAGCTGGTTTCT-3′ | 18 |
| having 171 and 161 nt HAs from TRAC | | |
| gene exon 1 | | |
| Reverse primer for generating donor DNA | p-5′-GACCTCATGTCTAGCACAGTTTTG-3′ | 19 |
| having 171 and 161 nt HAs from TRAC | | |
| gene exon 1 | | |
| Forward primer for generating donor DNA | 5′-ATCACGAGCAGCTGGTTTCT-3′ | 82 |
| having 171 and 161 nt HAs from TRAC | | |
| gene exon 1 - unmodified | | |
| Reverse primer for generating donor DNA | 5′-GACCTCATGTCTAGCACAGTTTTG-3′ | 21 |
| having 171 and 161 nt HAs from TRAC | | |
| gene exon 1 - unmodified | | |
| Forward primer for generating donor DNA | 5′-T*Am*T*GmCmACAGAAGCTGCAAGG-3′ | 28 |
| having 183 and 140 nt HAs from TRAC | | |
| gene exon 3 | | |
| Reverse primer for generating donor DNA | p-5′-TTAGGATGCACCCAGAGACC-3′ | 29 |
| having 183 and 140 nt HAs from TRAC | | |
| gene exon 3 | | |
| Forward primer for generating donor DNA | p-5′-CTCCCCATCTCCTCTGTCTC-3′ | 34 |
| having 326 and 380 nt HAs from PD-1 | | |
| locus | | |
| Reverse primer for generating donor DNA | 5′-Cm*Cm*T*GmACCCGTCATTCTACAG-3′ | 35 |
| having 326 and 380 nt HAs from PD-1 | | |
| locus | | |
| Forward primer for generating donor DNA | 5′-TGGAGCTAGGGCACCATATT-3′ | 36 |
| having 660 and 650 nt HAs from TRAC | | |
| gene exon 1 - unmodified | | |
| Forward primer for generating donor DNA | 5′-ATCACGAGCAGCTGGTTTCT-3′ | 37 |
| having 171 and 161 nt HAs from TRAC | | |
| gene exon 1 | | |
| Reverse primer for producing donor DNA | 5′-Gm*Cm*Am*CTGTTGCTCTTGAAGTCC-3′ | 54 |
| having 192 and 159 nt HAs from TRAC | | |
| gene exon 1 | | |
| Forward primer for generating DAR donor | p-5′-TGGAATACAGAGCGGAGGTC-3′ | 61 |
| DNA having 171 and 161 nt HAs from | | |
| Tim-3 gene | | |
| Reverse primer for generating donor DNA | 5′-Gm*Cm*Am*TGCAAATGTCCACTCAC-3′ | 62 |
| having 192 and 159 nt HAs from TRAC | | |
| gene exon 1 | | |
| forward primer producing donor fragment | 5′-Cm*T*Gm*CmAGGGAGGACATTCTCT-3′ | 88 |
| having 212 and 170 nt HAs from CD7 | | |
| locus | | |
| reverse primer producing donor fragment | 5′-p-TTCCCTACTGTCACCAGGA-3′ | 89 |
| having 212 and 170 nt HAs from CD7 | | |
| locus | | |

### Example 1. Simultaneous Knockout of the T-Cell Receptor Gene and Knock-In of Andi-CD38 CAR in Human T Cells

In this example, the T cell receptor alpha constant (TRAC) gene (Entrez Gene ID: 28755) was targeted with an anti-CD38 CAR construct as the donor DNA. The pAAV-TRAC-anti-CD38 construct was designed with approximately 1.3 kb of genomic DNA sequence of the T cell receptor alpha constant (TRAC) that flanks the target sequence (CAGGGTTCTGGATATCTGT (SEQ ID NO: 1)) in the genome. The target sequence was identified as a site upstream of a Cas9 PAM (GGG) in exon 1 of the TRAC gene for Cas9-mediated gene disruption and insertion of the donor construct. The anti-CD38 CAR gene construct (SEQ ID NO:2) comprised a sequence encoding a single chain variable fragment (scFv) specific for human CD38, followed by a CD8 and CD28 hinge domain-CD28 transmembrane domain-CD28 intracellular regions and a CD3 zeta intracellular domain. An exogenous JeT promoter (U.S. Pat. No. 6,555,674; SEQ ID NO:3) was used to initiate transcription of the anti-CD38 CAR.

To construct the pAAV-anti-CD38A2 donor plasmid which was used as a PCR template for generating donor DNA fragments genome editing, the anti-CD38A2 CAR construct with 650-660 bp homology arms (SEQ ID NO:4) was synthesized by Integrated DNA Technologies (IDT, Coralville, Iowa). An in-fusion cloning reaction was performed at room temperature, containing the pAAV-MCS vector double digested with MluI and BstEII (50 ng), the anti-CD38A2 CAR fragment with flanking homology arms (SEQ ID NO:4) (50 ng), 1 ul 5× In-Fusion HD Enzyme Premix (Takara Bio), and nuclease-free water. The reaction was briefly vortexed and centrifuged prior to incubation at 50° C. for 30 min. Stellar™ Competent Cells (Takara Bio USA) were then transformed with the in-fusion product and plated on ampicillin-treated agar plates. Multiple colonies were chosen for Sanger sequencing (Genewiz, South Plainfield, N.J.) to identify the correct clones using the sequencing primers CTTAGGCTGGGCATAGCAG (SEQ ID NO:5), CATGGAATGGTCATGGGTCT (SEQ ID NO:6), and GGCTACGTATTCGGTTCAGG (SEQ ID NO:7). Correct clones were cultured and the DNA plasmids from these clones were purified.

For RNA guide-directed targeting of the TCR alpha (TRAC) gene, the tracr RNA (ALT-R® CRISPR-Cas9 tracrRNA) and crRNA (ALT-R® CRISPR-Cas9 crRNA) were purchased from IDT (Coralville, Iowa), where the crRNA was designed to include the target sequence CAGGGTTCTGGATATCTGT (SEQ ID NO:1) that occurs directly upstream of a Cas9 PAM sequence (GGG) in first exon of the TRAC gene.

To make donor fragment DNA, PrimeSTAR Max Premix (Takara Bio USA) was used for PCR reactions. The AAV donor plasmid pAAV-anti-CD38A2 described above was used as a template. To generate a donor fragment with homology arms of 660 nt (SEQ ID NO:44) and 650 nt (SEQ ID NO:45), the forward primer had the sequence: TGGAGCTAGGGCACCATAT (SEQ ID NO:36), and the reverse primer had the sequence: CAACTGGAGAAGGGGCTTA (SEQ ID NO:9). In various experiments to test the effectiveness of different homology arm lengths, primers having sequences hybridizing to specific positions within the homology arms of the pAAV-anti-CD38A2 construct were used to produce donor fragments with homology arms of desired lengths by PCR. Phosphorothioate bonds (FIG. 2A) were introduced into the terminal three nucleotides at the 5′-end of the forward primer (SEQ ID NO:36) to inhibit exonuclease degradation (between the first and second, second and third, and third and fourth nucleotides from the 5′ terminus). The nucleotides at the second, third and fourth positions from the 5′-end of the forward oligonucleotide primer were also 2′-O-methyl modified (FIG. 2B) (SEQ ID NO:8, see Table 1). The reverse primer (SEQ ID NO:9) included a 5′-end phosphate. To produce the donor DNA fragment, the thermocycler settings were: one cycle of 98° C. for 30 s, cycles of 98° C. for 10 s, 64 to 66° C. for 5 to 15 s, 72° C. for 30 s and one cycle of 72° C. for 7 to 10 min. Digestion with a strandase to generate a single-stranded template was done using the Guide-it™ Long ssDNA Production System kit (Takara Bio USA) according to the manufacturer's instructions (Takara Bio USA), and ssDNA was purified using the NucleoSpin Gel and PCR Clean-Up kits (Takara Bio USA). The concentration of ssDNA was determined by NanoDrop (Denovix, Wilmington, Del.). As controls, donor fragments were produced with unmodified primers, such that the resulting donor fragment had no chemical modifications (no PS or 2′-O-methyl groups) or with a forward primer that had the PS modifications only (no 2′-O-methyl groups).

To generate TCR knockouts/anti-CD38 CAR knock-ins, T cells were activated by adding CD3 to the cultures. Approximately 48 to 72 hours after initiating T-cell activation with CD3, the PBMC cultures including activated T cells were electroporated with an SpCas9 ribonucleoprotein complex (RNP) that included SpCas9 protein (that included nuclear localization sequences; IDT) plus crRNA (including guide sequence SEQ ID NO:1) and tracrRNA using a Neon® Transfection System (ThermoFisher Scientific) and 10 μl or 100 μl tips. Briefly, Alt-R® CRISPR-Cas9 crRNA and Alt-R® tracrRNA (both from IDT) were first mixed and heated at 95° C. for 5 min. The mixture was then removed from heat and allow to cool to room temperature (15-25° C.) on the bench top for about 20 min to make a crRNA:tracrRNA duplex. For each transfection, 10 μg SpCas9 protein (IDT) was mixed with 200 pmol crRNA:tracrRNA duplex and incubated together at 4° C. for 30 min to form RNPs. 1×10⁶ cells were mixed with the RNP and electroporated with 1700 V, 20 ms pulse width, 1 pulse. One to two hours later, 10 ug single-stranded donor DNA was electroporated into the cells with 1600 V, ms pulse width, 1 pulse. In some cases, T cells were mixed with the RNP and the donor DNA and RNP plus donor DNA were electroporated into the cells at the same time. Following electroporation cells were diluted into culture medium and incubated at 37° C., 5% CO₂.

To determine knock-in efficiency by detecting CAR expression of transformed cells by FACS, transfected or retrovirally transduced PBMCs were washed with DPBS/5% human serum albumin, then stained with anti-CD3-BV421 antibody SK7 (BioLegend) and PE conjugated anti-CD38-Fc protein (Chimerigen Laboratories, Allston, Mass.) for 30-60 min at 4° C. CD3 and anti-CD38 CAR expression were analyzed using iQue Screener Plus (Intellicyte Co.) Negative controls for the anti-CD38 construct knock-in were cells that had been transfected with an RNP that included Cas9 protein complexed with a hybridized tracrRNA and crRNA targeting the first exon of the TRAC gene, but that had not been transfected with the anti-CD38 CAR donor DNA. As controls for expression of the anti-CD38 CAR construct by knock-in cells, anti-CD38 CAR-expressing PBMCs were generated by non-Cas9 methods. PBMCs that had been transfected with the RNP that included the guide targeting the TRAC locus (TCR knockout cells) but no donor DNA were transduced with a retrovirus that included a retroviral vector having the same anti-CD38A2 expression cassette (SEQ ID NO:2) that was used to make the donor fragment employed in CRISPR/Cas9 targeting.

FIGS. 3A to 3E show that 8 days after transfection no expression of an anti-CD38 construct was detected in cells transfected with the RNP (for knocking out the TRAC gene) in the absence of a donor fragment for expression of the anti-CD38 CAR (FIG. 3A). On the other hand, PBMCs that had a TRAC knockout and were subsequently transduced with a retrovirus that included a construct for expressing the anti-CD38 CAR did show expression of the anti-CD38 CAR in about 70% of the cells 8 days after transfection (FIG. 3E). For cultures transformed with anti-CD38 CAR ss donor DNA in addition to an RNP targeting exon 1 of the TRAC gene, approximately 12% of the population that received the ss donor DNA having no chemical modifications and approximately 13% of cultures that were transduced with ss donor DNA having only PS backbone modifications on nucleotides near the 5′-end of the donor DNA (introduced by using a PCR primer having PS bonds between nucleotides 1 and 2, 2 and 3, and 3 and 4, numbering from the 5′ end) demonstrated expression of the anti-CD38 construct. Adding methyl groups to the 2′ oxygen of the three nucleotides at the second, third, and fourth nucleotides from the 5′-end of the donor fragment strand that also included PS modifications (by using the primer of SEQ ID NO:8 that included these modifications to generate the donor DNA by PCR) resulted in significantly higher expression of the anti-CD38 CAR in the transfected population, where expression of the anti-CD38 CAR was seen in approximately 20% of the cells that received the ‘double modified’ (2′-O-methyl and PS) single-stranded donor fragment at 8 days. Notably, chemical modifications of the donor DNA did not affect viability of the transfected cultures.

Increased expression of the anti-CD38 CAR was observed over time in cultures that had been transfected with anti-CD38 CAR donor fragments plus RNPs targeting the TRAC gene, while the opposite was true of cultures transduced by a retrovirus. At 10 days post-transfection (FIGS. 3F to 3J), flow cytometry demonstrated that for all of the cultures—that were all transfected with the TRAC-targeting RNP—at least 80% of the cells did not express the TCR. Moreover, in cultures transfected with an anti-CD38 CAR donor fragment in addition to the TRAC-targeting RNP, at least 42% of the cells that did not express the TCR expressed the anti-CD38 construct (FIGS. 3G to 3I) at 10 days post-transfection. For cultures transfected with an anti-CD38 CAR donor fragment with both PS and 2′-O-methyl groups on 5′-proximal nucleotides, 57% of the cells were expressing the anti-CD38 construct by day ten (FIG. 3I), the highest percentage of any test culture. At the same time, the expression of the anti-CD38 CAR in cultures that had been transduced with the retrovirus dropped to about half of what had been seen at 8 days, to approximately 34% of the cells on day ten post-transduction (FIG. 3J). Analysis of the culture transfected with doubly modified ss donor and the retrovirus-transduced culture at day 20 (FIGS. 3K to 3M) showed that expression of the anti-CD38 construct in the cultures had stabilized, with the Cas9-modified culture that had been transfected with a ss donor having both PS and 2′-O-methyl modifications at the 5′ end demonstrating 54% of the TCR-negative cells were expressing the construct (FIG. 3L) and the culture that had been transduced with a retrovirus demonstrating 31% of the TCR-negative cells were expressing the construct (FIG. 3M).

To confirm the occurrence of homology directed repair (HDR) at the targeted locus in Exon 1 of the TRAC gene, PCR was performed on DNA isolated from cultures to verify that the donor fragment had inserted into the TRAC site targeted by the guide RNA. Genomic DNA was amplified from non-transfected activated T cells (ATCs), TRAC knockout cells that were transformed with the RNP that included the TRAC Exon 1 guide RNA, and from T cells transfected with the RNP plus phosphorothioate and 2′ O-Methyl modified donor DNA to detect targeted insertion of an anti-CD38 CAR transgene into the TRAC locus. To confirm the position of the donor DNA in the genome, oligonucleotide primers were targeted to sequences outside of the TRAC homology arms but adjacent to (outside of) the homology arm sequences in the genome. A total of 1×10⁵ cells were resuspended in 30 μL of Quick Extraction solution (Epicenter) to extract the genomic DNA. The cell lysate was incubated at 65° C. for 5 min and then at 95° C. for 2 min and stored at −20° C. The concentration of genomic DNA was determined by NanoDrop (Denovix). Genomic regions containing the TRAC target sites were PCR-amplified using the following primer sets: 5′ PCR forward primer on TRAC: CCTGCTITCTGAGGGTGAAG (SEQ ID NO:10), 5′ PCR Reverse primer on CAR: CTITCGACCAACTGGACCTG (SEQ ID NO:11); 3′ Forward primer on CAR: CGTTCTGGGTACTCGTGGTT (SEQ ID NO:12), 3′ Reverse primer on TRAC: GAGAGCCCTTCCCTGACTIT (SEQ ID NO:13) (see FIG. 1B). Both primer sets were designed to avoid amplifying the HDR templates by annealing outside of the homology arms.

The concentration of genomic DNA was determined by NanoDrop (Denovix). Both primer sets were designed such that one primer of the pair annealed to a site in the genome outside of the homology arm, and the other primer of the pair annealed to a site within the coding region of the construct (i.e., not in a homology arm). The PCR contained 400 ng of genomic DNA and Q5 high fidelity 2× mix (New England Biolabs). The thermocycler setting consisted of one cycle of 98° C. for 2 min, 35 cycles of 98° C. for 10 s, 65° C. for 15 s, 72° C. for 45 s and one cycle of 72° C. for 10 min. The PCR products were purified on 1% agarose gel containing SYBR Safe (Life Technologies). The PCR products were then eluted from the agarose gel and isolated using NucleoSpin® Gel and PCR Clean-up kit (MACHEREY-NAGEL GmbH & Co. KG). The PCR products were submitted for Sanger sequencing (Genewiz). FIG. 4 provides a photograph of the gel separating PCR products. The positive bands corresponding to the anti-CD38 construct adjacent to genomic sequences adjacent to the homology arms in the genome at the 5′ and 3′ ends of the construct were only seen in cells transfected with donor DNA (lanes 3 and 6) and not in non-transfected ATCs (lanes 1 and 4) or TRAC knock out-only cells (lanes 2 and 5). Sequencing of these PCR products confirmed that the anti-CD38 CAR construct inserted at the predicted site, where the PCR fragments generated from the genomic DNA of cells having the integrated anti-CD38 CAR construct using the primers annealing to genomic sequences outside the region of the homology arms and to construct sequences inside the homology arm sequences at both the 5′ and 3′ ends of the constructs had the predicted sequences for constructs integrated at the targeted site. Sequencing of PCR products produced using primers to diagnose the insertion locus (see FIG. 1B) provided sequences demonstrating the anti-CD38 CAR donor fragment integrated into exon 1 of the TRAC gene. PCR product sequences (e.g., SEQ ID NO:39 and SEQ ID NO:40) included sequences adjacent to (outside of) the homology arm in the genome, sequence of the homology arm present in the donor fragment, and portions of the anti-CD38 CAR sequence in a single PCR product, demonstrating the insertion at the expected site.

To test for function of transfected cells, three weeks after electroporation, the activated T cells that had been transfected with the anti-CD38 CAR targeted to the TRAC locus were starved with IL-2 overnight and tested in specific killing assays. The activated T cells were co-cultured with a target cell mixture of CD38 positive RPMI-8226/GFP cells and CD38 negative K562/RPE cells. The incubation effector-to-target cell ratio ranged from 10:1 to 0.08:1. After overnight incubation, the cells were analyzed by flow cytometry to measure the GFP-positive and RPE-positive cell populations to determine the specific target cell killing by anti-CD38A2 CART cells. FIG. 5 provides a graph of the specific cytotoxicity of each cell population against CD38-expressing RPMI8226 cells (the observed cytotoxicity against CD38-expressing RPMI8226 cells after subtracting out the observed cytotoxicity toward K562 cells that do not express CD38). The graph shows that while non-transfected ATC cells showed some toxicity at the highest effector to target ratios, TRAC knockout cells showed virtually no killing regardless of effector-to-target cell ratio. The anti-CD38A2 CART cells however exhibited potent and specific killing activity of CD38 positive cells—RPMI8226, but not CD38 negative cells—K562 is (FIG. 5). T cells that had integrated the chemically modified donor that included the anti-CD38 CAR cassette demonstrated cytotoxicity toward target cells similarly to that of cells transduced with retrovirus that included the anti-CD38 CAR construct.

The transfected activated T cells (ATCs) were also tested for cytokine secretion (FIGS. 6A to 6C). T cells were starved in IL-2 free medium overnight. Anti-CD38 CAR-T cells or ATC controls were then co-cultured with CD38 negative K562 or CD38 positive RPMI8226 cells. The incubation effector to target cell ratio was 2:1. After overnight incubation, the cells were centrifuged to collect the supernatants for quantitating cytokine IL-2, IFN-gamma and TNF alpha (Affymetrix eBioscience) according to the manufacturer's instructions. The gene-edited TCR knockout anti-CD38A2 CART cells also released similar amount of IFN-γ and other pro-inflammatory cytokines when co-cultured with CD38 positive tumor cells (RPMI8226) but not CD38 negative cells (K562).

In summary, in vitro cellular functional studies did not reveal any notable differences between TRAC-site-specific integrated anti-CD38A2 CAR achieved by this novel and efficient process and virus-mediated randomly integrated anti-CD38A2 CAR, in terms of both specific killing assay (FIG. 5) and cytokine secretion assay (FIGS. 6A to 6C).

### Example 2. Reducing Length of Homology Arms of Donor DNAs

For knock-in of the anti-CD38 CAR construct, donor fragments having homology arms (HAs) of different lengths were produced and tested. The pAAV-TRAC-anti-CD38 construct described in Example 1 that included the anti-CD38 cassette plus TRAC exon 1 homology arms of 660 and 650 nts (SEQ ID NO:4) was used as the template. A first set of primers, SEQ ID NO:8 and SEQ ID NO:9, was used to generate a donor fragment having homology arms of 660 nt and 650 nt from this template as provided in Example 1. A second set of primers, SEQ ID NO:14 and SEQ ID NO:15, was used to generate a donor fragment having homology arms of approximately 350 nt (375 and 321 nucleotides), where the primer of SEQ ID NO:14 had PS linkages between the between first and second, second and third, and third and fourth nucleotides from the 5′ terminus and had 2′-O-methyl-modified nucleotides at positions 2, 3, and 5. A third set of primers, SEQ ID NO:18 and SEQ ID NO:19, was used to generate a donor fragment having homology arms of approximately 165 nt (171 and 161 nts), where the primer of SEQ ID NO:18 had PS linkages between the between first and second, third and fourth, and fourth and fifth nucleosides from the 5′ terminus and had 2′-O-methyl-modified nucleotides at positions 3, 4, and 5. In each case, the forward primer (SEQ ID Nos: 8, 14, and 18) was designed to have three PS linkages within the 5′terminal-most five nucleotides (for example, between any of the first and second, second and third, third and fourth, and fourth and fifth nucleosides from the 5′ terminus of the primer, and three 2′-O-methyl groups occurring in any of the five 5′terminal-most nucleotides. In each case, the reverse primer (SEQ ID Nos: 9, 15, and 17) had a 5′ terminal phosphate (see Table 1).

Each of the primer sets was used with the pAAV CD38 DAR construct as a template to generate a donor DNA molecule having multiple PS and 2′-O methyl modifications proximal to the 5′end of one strand of the donor and a 5′ phosphate at the 5′ terminus of the opposite strand of the donor. RNPs were assembled to include tracr and crRNAs as described in Example 1, where the crRNA included the target sequence of SEQ ID NO:1, a sequence found in exon 1 of the TRAC gene. When synthesizing donor DNA by PCR, the nuclease reaction to generate the single stranded donor fragment and subsequent purification of the single stranded DNA are time consuming and typically result in significant losses in the yield of donor fragment for transfections. In addition, the nuclease reaction can be difficult to control so that the ends of the donor fragments can be degraded. In further experiments testing the efficiency of directed gene knockouts and antibody construct knock-ins, double-stranded donor DNAs were tested in transfections to eliminate the nuclease digestion and single-strand purification of the PCR-synthesized donor.

The double-stranded donor molecules, having homology arms of approximately 665, 350, and 165 base pairs in length, were independently transfected into activated T cells as described in Example 1 except that donor fragments and RNPs were transfected in the same electroporation under conditions for electroporating the RNP (using a Neon® Transfection System (ThermoFisher Scientific) 1700 V, 20 ms pulse width, 1 pulse). As a control, activated T cells were transfected with the RNP in the absence of a donor fragment, which should result in knockout of the targeted TRAC locus, but without donor DNA insertion. To test for expression of the T cell receptor and the anti-CD38 CAR construct, flow cytometry was performed as provided in Example 1. FIGS. 7A to 7D shows that, as expected, the T cell culture transfected with the RNP only had low levels of expression of the T cell receptor and also demonstrated no expression of the anti-CD38 CAR. T cells transfected with the RNP plus donor DNAs having homology arms of different sizes however show low levels of T cell receptor expression and good expression of anti-CD38 CAR in the cultures, demonstrating that transfection of a double-stranded donor DNA is highly effective for targeted knock-ins. Further, surprisingly, the shortest HA lengths tested, 161/171 nt, worked better than longer HA lengths, with the percentages of knockout cells expressing the introduced construct being approximately 24% for approximately 665 nt arms, approximately 30% for approximately 350 nt arms, and approximately 38% for approximately 165 nt arms. The short homology arms are thus found to be very effective in targeted knock-in genome modification using double-stranded DNA donors, which has the benefit of allowing for smaller constructs and/or allowing for more capacity in a construct to allow inclusion of additional or lengthier sequences to be included in the donor DNA.

### Example 3. Modified Versus Non-Modified Double-Stranded Donor DNA

Donor DNAs that included anti-CD38 CAR and having the approximately 165 nt TRAC exon 1 homology arms as set forth in Example 2, above, were synthesized using primers with and without nucleotide modifications to test their relative effectiveness in promoting HDR. In the first case, primer SEQ ID NO:18 had three PS linkages, occurring between first and second, third and fourth, and fourth and fifth nucleosides and three 2′-O-methyl-modified nucleotides within the first five nucleotides of the 5′ terminus of the primer (at nucleotide positions 3, 4, and 5) and primer SEQ ID NO:19 had a 5′ terminal phosphate (Table 1). These primers were used to generate a donor DNA with HAs of 171 bp and 161 bp, respectively, and with the corresponding nucleotide modifications (i.e., three PS linkages and three 2′-O-methyl groups within five nucleotides of the 5′ terminus of the first strand of the donor DNA product, and a phosphate on the 5′ end of the second strand of the donor DNA product). In the second case, primer SEQ ID NO:37 was identical to primer SEQ ID NO:18 except that primer SEQ ID NO:37 lacked chemical modifications see Table 1). The SEQ ID NO:37 primer and the SEQ ID NO:19 primer lacking a 5′ terminal phosphate was used to generate a donor DNA with no nucleotide modifications having the anti-CD38 CAR cassette. These donor DNAs were transfected as double-stranded DNA molecules (with no denaturation or nuclease digestion of either strand) along with RNPs that included a trRNA and a crRNA that included the target sequence of SEQ ID NO:1 (within exon 1 of the TRAC gene) into activated T cells. In the electroporations with double stranded DNA as donor, 5 ug dsDNA was used to transfect one million activated T cells.

As in Example 2, control activated T cells were transfected with the RNP in the absence of a donor fragment, which should result in knockout of the targeted TRAC locus without construct insertion. To test for expression of the T cell receptor and the anti-CD3 CAR construct, flow cytometry was performed essentially as provided in Example 1. The results, shown in FIGS. 8A to 8C, show that transfection with the RNP and a modified double stranded donor resulted in greater than 50% of the cells expressing the anti-CD38 CAR while demonstrating no TCR expression, at least twice the percentage of TCR negative cells expressing the anti-CD38 construct as observed in the culture transfected with the RNP and the unmodified double-stranded donor (22%).

### Example 4. HDR-Mediated Knock-In of Anti-CD19 and Anti-BCMA CAR Constructs with Simultaneous TCR Knockout

Additional donor DNAs that included anti-CD19 CAR and anti-BCMA CAR expression constructs were also tested for insertion into the TRAC locus.

An anti-CD19 CAR construct that included an anti-CD19 CAR cassette (SEQ ID NO:22) that included the Jet promoter (SEQ ID NO:3) and intron, an anti-CD19 CAR construct, and an SV40 polyA sequence was made essentially as described for the anti-CD38 CAR pAAV construct described in Example 1 and was cloned in a vector flanked by the TRAC gene exon 1 homology arms (HAs) of SEQ ID NO:20 and SEQ ID NO:21. The anti-CD19 CAR with HAs pAAV construct was used as a template in PCR reactions as provided in Example 1 using the primers provided as SEQ ID NO:18 and SEQ ID NO:19 that result in the production of modified donor DNA having HAs of approximately 170 and 160 nucleotides (see Table 1). The forward primer (SEQ ID NO:18) had three PS bonds between the first and second, third and fourth, and fourth and fifth nucleosides and three 2′-O-methyl modifications at nucleotides 3, 4, and 5 when numbering from the 5′-terminus of the primer and the reverse primer (SEQ ID NO:19) had a 5′-terminal phosphate (Table 1). The resulting double-stranded donor DNA was therefore synthesized to have the corresponding modifications, a first strand with three PS and three 2′-O-methyl modifications within five nucleotides of the 5′-terminus, and a second strand with a 5′-terminal phosphate.

The double-stranded chemically modified donor fragment having the sequence of SEQ ID NO:38 with the nucleotide modifications of primers SEQ ID NO:18 and SEQ ID NO:19 described above incorporated was used to transfect cells along with an RNP that was produced according to the methods provided in Example 1, where the crRNA of the RNP included the target sequence of SEQ ID NO:1, targeting exon 1 of the TRAC gene. As a control, activated T cells were transfected with the RNP in the absence of a donor fragment, which should result in knockout of the targeted TRAC locus without construct insertion. Flow cytometry was performed essentially as described in Example 1 to evaluate the efficiency of introducing a different construct into the TRAC locus, except that anti-CD19 CAR expression was detected by CD19-Fc (Speed Biosystem) followed by APC anti-human IgG Fcγ (Jackson Immunoresearch). The results are shown in FIGS. 9A to 9B, where it can be seen that the anti-CD19 CAR was expressed in the absence of T cell receptor expression in approximately 42% of the cells in the culture.

An anti-BCMA CAR construct was made through replacing the anti-CD38 CAR with an anti-BCMA CAR based on the anti-CD38 CAR pAAV construct described in Example 1. The anti-BCMA CAR fragment was synthesized by IDT. The sequence of the insert is provided as SEQ ID NO:23. The anti-BCMA CAR construct was used as a template in PCR reactions as set forth in Example 1 using the primers provided as SEQ ID NO:18 and SEQ ID NO:19 that result in the production of donor DNA having TRAC Exon 1 locus HAs of approximately 160-170 nucleotides (see Table 1). The forward primer (SEQ ID NO:18) had three PS and three 2′-O-methyl modifications within five nucleotides of the 5′-terminus of the primer. The reverse primer (SEQ ID NO:19) had a 5′-terminal phosphate. The resulting double-stranded donor DNA was therefore synthesized to have a first strand with three PS and three 2′-O-methyl modifications within five nucleotides of the 5′-terminus, and a second strand with a 5′-terminal phosphate.

The double-stranded donor fragment having the sequence of SEQ ID NO:37, having modified nucleotides by incorporation of chemically modified primers as provided above, was used to transfect cells along with an RNP that was produced according to the methods provided in Example 1, where the crRNA of the RNP included the target sequence of SEQ ID NO:1, targeting exon 1 of the TRAC gene. As a control, activated T cells were transfected with the RNP in the absence of a donor fragment, which should result in knockout of the targeted TRAC locus without construct insertion. Flow cytometry was performed as described in Example 1 to evaluate the efficiency of introducing a different construct into the TRAC locus, except that anti-BCMA CAR expression was detected by PE or APC conjugated BCMA-Fc (R&D). The results are shown in FIGS. 10A to 10B, where it can be seen that the anti-BCMA CAR was expressed in the absence of T cell receptor expression in approximately 66% of the cells in the culture.

### Example 5. HDR Mediated Knock-In Targeting TRAC Exon 3

To test the efficiency of inserting donor DNAs into additional loci using the methods for donor insertion provided herein, an anti-CD38 CAR construct was made for producing a donor DNA having HAs from Exon 3 of the TRAC gene. In this case, the construct was produced essentially as described in Example 1 for the TRAC exon 1 targeting construct, except that the HAs (5′ HA SEQ ID NO:24 (183 nt) and 3′ HA SEQ ID NO:25 (140 nt)) were sequences surrounding the exon3 target site (SEQ ID NO:26). The sequence of the insert of the pAAV construct that was then produced as a donor DNA with TRAC gene exon 3 homology arms is provided as SEQ ID NO:27. To generate the donor fragment, the forward primer (SEQ ID NO:28) included PS linkages between first and second, second and third, and third and fourth nucleosides and 2′-O-methyl modifications on the second, fourth, and fifth positions from the 5′-terminus, and the reverse primer (SEQ ID NO:29) had a 5′-terminal phosphate. The resulting double-stranded donor DNA that incorporated the primers had a first strand with corresponding PS and 2′-O-methyl modifications on the 5′-terminal most nucleotides, and a second strand having a 5′-terminal phosphate.

The double-stranded donor fragment having modified nucleotides by incorporation of the primers above and having the sequence of SEQ ID NO:27 was used to transfect cells along with an RNP that was produced according to the methods provided in Example 1, where the crRNA included the target sequence of SEQ ID NO:26, targeting exon 3 of the TRAC gene. As a control, activated T cells were transfected with the RNP in the absence of a donor fragment, which should result in knockout of the targeted TRAC locus without construct insertion. A further control was non-transfected activated T cells (ATCs). Flow cytometry was performed essentially as described in Example 1. The results are shown in FIGS. 11A to 11C, where it can be seen that transfection with the RNP or the RNP plus donor DNA result in greater than 80% of cells across the culture losing TCR expression. Further, anti-CD38 CAR was expressed in the absence of T cell receptor expression in approximately 42% of the cells in the culture that was transfected with the targeting RNP plus the donor DNA with HAs derived from the TRAC gene exon 3.

PCR products were generated using primers designed to diagnose the insertion locus (see FIG. 1B): 5′-CTCCTGAATCCCTCTCACCA-3′ (SEQ ID NO:64, forward primer for sequencing across 5′ homology arm of anti-CD38 CAR in the TRAC exon 3 locus) and 5′-GCGGATCCAGCTCATGTAGT-3′ (SEQ ID NO:65, reverse primer for sequencing across 5′ homology arm of anti-CD38 CAR in TRAC exon 3 locus) and for the opposite junction, 5′-CGTTCTGGGTACTCGTGGTT-3′ (SEQ ID NO:66, forward primer for sequencing across 3′ homology arm of anti-CD38 CAR in TRAC exon 3 locus) and 5′-GGAGCACAGGCTGTCTTACA-3′ (SEQ ID NO:67, reverse primer for sequencing across 3′ homology arm of anti-CD38 CAR in TRAC exon 3 locus). The resulting PCR products were sequenced. The PCR product sequences (e.g., SEQ ID NO:41 and SEQ ID NO:42) included sequences adjacent to the homology arm in the genome, the homology arm present in the donor is fragment, and portions of the anti-CD38 CAR in a single PCR product, demonstrating the expected insertion.

FIGS. 12A to 12D compares targeting of the anti-CD19 CAR to exon 3 and exon 1 of the TRAC gene. The anti-CD19 CAR donor DNA directed to exon 3 is synthesized to include the anti-CD19 CAR cassette (SEQ ID NO:22) as set forth in the Examples above, where the anti-CD19 expression cassette is flanked by sequences from the exon 3 locus (SEQ ID NO:24 and SEQ ID NO:25) as set forth above. The anti-CD19 CAR donor directed to exon 1 (having the sequence of SEQ ID NO:38) is provided in Example 4. Each of these constructs—one having the anti-CD19 CAR cassette (SEQ ID NO:22) flanked by TRAC exon 1 HAs (SEQ ID NO:18 and SEQ ID NO:19), and the other having the anti-CD19 CAR cassette (SEQ ID NO:22) flanked by TRAC exon 3 HAs (SEQ ID NO:24 and SEQ ID NO:25), was used to produce donor fragment using modified forward primers having PS and 2′-O-methyl modifications on the three 5′-terminal most nucleotides. The reverse primers had 5′-terminal phosphates. The primers for producing the anti-CD19 CAR donor flanked by exon 1 HAs were SEQ ID NO:18 and SEQ ID NO:19, where the SEQ ID NO:18 primer included PS linkages between first and second, third and fourth, and fourth and fifth nucleosides and 2′-O methyl groups at position 3, position 4, and position 5 from the 5′ end. The primers for producing the anti-CD19 CAR donor flanked by exon 3 HAs were SEQ ID NO:28 and SEQ ID NO:29, where the SEQ ID NO:28 primer had PS linkages between the first and second, second and third, and third and fourth nucleosides from the 5′ end and 2′-O-methyl groups at position 2, position 4, and position 5 from the 5′ end. The resulting double-stranded donor DNAs thus had a first strand with corresponding PS and 2′-O-methyl modifications on the 5′-terminal end nucleotides, and a second strand having a 5′-terminal phosphate.

The donor fragments were independently transfected into activated T cells with RNPs. RNPs were produced as described in Example 1, where the target sequence of the crRNA for targeting TRAC gene exon 1 was SEQ ID NO:1, and the target sequence of the crRNA for targeting TRAC gene exon 3 was SEQ ID NO:26. As can be seen in FIGS. 12A to 12D, approximately 41% of the culture that was transfected with an RNP targeting exon 3 of the TRAC gene and a donor fragment for expressing the anti-CD19 CAR were both TCR negative and positive for anti-CD19 CAR, while approximately 20% of the culture that was transfected with an RNP targeting exon 1 of the TRAC gene and a donor fragment for expressing the anti-CD19 CAR were both TCR negative and positive for anti-CD19 CAR. T cell cultures transduced with a retrovirus including the anti-CD19 CAR expression cassette demonstrated a higher percentage of anti-CD19 CAR expressing cells, but these cells did not have a TCR knockout.

### Example 6. HDR Mediated Knock-In Targeting the PD-1 Gene

The PD-1 locus was also targeted with a CAR construct. In this case the anti-CD38 CAR cassette (SEQ ID NO:2) was juxtaposed with homology arms (SEQ ID NO:30 and SEQ ID NO:31) having sequences of the PD-1 locus that surround a target site (SEQ ID NO:32) using the methods essentially as described in Example 1 to provide a template for producing donor DNA.

Donor DNA was produced essentially as described in Example 1, using a forward primer (SEQ ID NO:34) that included a 5′ phosphate and a reverse primer that included phosphorothioate linkages between first and second, second and third, and third and fourth nucleosides from the 5′ end as well as 2′-O-methyl groups on the first, second, and fourth nucleosides from the 5′ end (SEQ ID NO:35), see Table 1.

The double-stranded chemically modified donor fragment (SEQ ID NO:33) was used to transfect cells along with an RNP produced according to the methods provided in Example 1, where the crRNA included the target sequence of SEQ ID NO:32, targeting the PD-1 gene. As a control, activated T cells were transfected with the RNP in the absence of a donor fragment, which generates a knockout of the targeted TRAC locus without CAR construct insertion. A further control was non-transfected activated T cells (ATCs). Flow cytometry was performed essentially as described in Example 1, where an additional control of nontransfected activated T cells (ATCs) was included. A BV421-conjugated antibody to PD-1 (EH12.2H7, BioLegend) was used to detect PD-1 expression.

The results are shown in FIGS. 13A to 13D, where it can be seen the percentage of cells expressing PD-1 dropped from approximately 19% in ATCs to approximately 4% in the cells of cultures transfected with the RNP targeting the PD-1 locus (PD-1 RNP). The anti-CD38 CAR was expressed in the absence of T cell receptor expression in approximately 27% of the cells in the culture that was transfected with the PD-1 targeting RNP plus a donor with HAs having homology to the PD-1 locus. As a comparison, about 32% of cells of a culture transfected with an RNP targeting exon 1 of the TCR and an anti-CD38 CAR donor fragment with HAs having homology to sequences of exon 1 of the TRAC gene.

Sequencing of PCR products produced using primers to diagnose the insertion locus (see FIG. 1B) provided sequences demonstrating the anti-CD38 CAR donor fragment integrated into the PD-1 gene. To obtain the junction sequences, a total of 1×10⁷ cells were resuspended in 500 μl of Quick Extraction solution (Epicenter) to extract the genomic DNA. The cell lysate was incubated at 65° C. for 5 min and then at 95° C. for 2 min and stored at −20° C. The concentration of genomic DNA was determined by NanoDrop (Denovix). Genomic regions, containing the target sites, were PCR-amplified. Primer sets for both the 5′ junction and 3′ junction were designed to anneal outside of the homology arms. PCR products were generated using primers designed to diagnose the insertion locus (see FIG. 1B): 5′-GTGTGAGGCCATCCACAAG-3′ (SEQ ID NO:68, forward primer for sequencing across 5′ homology arm of anti-CD38 CAR in the TRAC exon 3 locus) and 5′-ACACACTGCGACCCATTC-3′ (SEQ ID NO:69, reverse primer for sequencing across 5′ homology arm of anti-CD38 CAR in TRAC exon 3 locus) and for the opposite junction, 5′-CGTTCTGGGTACTCGTGGT-3′ (SEQ ID NO:70, forward primer for sequencing across 3′ homology arm of anti-CD38 CAR in TRAC exon 3 locus) and 5′-GGGACTGTCTTAGGCTTGG-3′ (SEQ ID NO:71, reverse primer for sequencing across 3′ homology arm of anti-CD38 CAR in TRAC exon 3 locus).

The PCR contained 400 ng of genomic DNA and Q5 High Fidelity 2×PCR mix (New England Biolabs). The thermocycler setting consisted of one cycle of 98° C. for 2 min, 35 cycles of 98° C. for 10 s, 65° C. for 15 s, 72° C. for 45 s and one cycle of 72° C. for 10 min. The PCR product were purified on 1% agarose gel containing SYBR Safe (Life Technologies). The PCR product were eluted from the agarose gel using NucleoSpin® Gel and PCR Clean-up kit (MACHEREY-NAGEL GmbH & Co. KG). The PCR product was submitted for Sanger sequencing (Genewiz). The PCR product sequences included sequences adjacent to the homology arm in the genome, the homology arm present in the donor fragment, and portions of the anti-CD38 CAR in a single PCR product, demonstrating the expected insertion.

FIG. 14 provides the results of cytotoxicity assays that were performed using PBMCs and isolated T cells from cultures transfected with the anti-CD38 CAR donor fragment and an RNP targeting the PD-1 locus (“PD-1 KOKI PBMC” and “PD-1 KOKI Tcell” respectively) to determine the functionality of cells expressing the anti-CD38 CAR and knocked out in the PD-1 gene. These modified cells showed a high level of cytotoxicity toward target cells in the assay with respect to control cells that had a PD-1 gene knockout but did not receive a CAR construct (“PD-1 KO”) and control cells that had a TRAC gene knockout but did not receive a CAR construct (“TRAC-1 KO”) and were outperformed somewhat by cells that were transfected the anti-CD38 CAR donor fragment and an RNP targeting the TRAC locus (“TRAC KOKI”), likely due to the lower efficiency of donor CAR construct integration at the PD-1 site that was observed (FIGS. 13A to 13D).

### Example 7. Targeted Insertion of an Anti-CD38 Dimeric Antibody Receptor (DAR) Construct into the TRAC Exon 1 Locus with Cas9 and Cas12a

In further experiments, further configurations of synthetic antibody-receptors were expressed in T cells. Constructs were made for the expression of dimeric antibody receptors (DARs, see, for example, WO 2019/173837, incorporated herein by reference), where the DAR constructs included a nucleic acid sequence encoding two polypeptides linked by a “self-cleaving” 2A sequence that was used to generate two polypeptides from a single open reading frame. The first encoded polypeptide was a heavy chain polypeptide that included a heavy chain variable region and the first heavy chain constant region (CH1), a hinge region, a transmembrane domain of CD28, and a cytoplasmic domain of 4-1BB and CDζ. This was followed by the Thosea asigna virus T2A peptide-encoding sequence (SEQ ID NO:46) and then by the sequence encoding the second polypeptide, where the second polypeptide included, proceeding from the N-terminus to the C-terminus, an immunoglobulin light chain variable region (VL) plus constant region (lambda). The nucleic acid sequences encoding the heavy chain polypeptide sequence, 2A peptide, and light chain sequence were operably linked to the JeT promoter (SEQ ID NO:3) at the 5′ end of the DAR-encoding sequence and an SV40 polyA addition sequence (SEQ ID NO:47) at the 3′end of the DAR-encoding sequence. The entire anti-CD38 DAR construct (JeT promoter heavy chain-encoding sequences with hinge, CD28 transmembrane domain, and cytoplasmic domains of 4-1BB and CDC, T2A, light chain, and SV40 sequence (SEQ ID NO:48)), was cloned between homology arms of 660 bp (SEQ ID NO:44) and 650 bp (SEQ ID NO:45) in a vector. The homology arms included sequences of the TRAC exon 1 locus on either side of the target sequence. Donor fragments for use in transfection experiments were synthesized by PCR using a forward primer that included three PS bonds between the first and second, third and fourth, and fourth and fifth nucleotides and three 2′-O-methyl modifications at nucleotides 3, 4, and 5 when numbering from the 5′-terminus of the primer (SEQ ID NO:18), and a reverse primer that included a 5′ terminal phosphate (SEQ ID NO:19) (Table 1). The resulting PCR product (SEQ ID NO:49) included the homology arms (SEQ ID NO:20 and SEQ ID NO:21) flanking the anti-CD38 DAR-encoding construct (SEQ ID NO:48) and had the primer modifications of SEQ ID NO:18 incorporated into the first strand and a 5′ terminal phosphate but no introduced chemical modifications added to the opposite, or second, strand.

The knock out/knock-in (“KOKI”) strategy was also tested with Cas12a, an RNA-guided endonuclease that does not use a tracrRNA and recognizes a PAM having the sequence TTV, where V is A, C, or G, where the PAM is immediately upstream of the target site. In these experiments, the same anti-CD38 DAR construct (SEQ ID NO:48) was cloned between homology arms, where the homology arm sequences (SEQ ID NO:50 and SEQ ID NO:51) had homology to genome sequences on either side of a Cas12a target site (SEQ ID NO:52) in exon 1 of the TRAC gene. The anti-CD38 DAR construct flanked by these homology sequences (SEQ ID NO:53) was cloned in a vector as described in Example 1 for the anti-CD38 CAR construct and the resulting clone was used as a template for PCR reactions using the forward primer SEQ ID NO:20, which included a 5′ terminal phosphate, and reverse primer SEQ ID NO:54 that had the first three nucleotides from the 5′ end 2′-O-methylated (2′-O-methyl deoxyguanosine, 2′-O-methyl deoxycytidine, and 2′-O-methyl deoxyadenosine) and where the first three nucleotides were linked to the next nucleotide via PS bonds (i.e., there were PS linkages between the first and second, second and third, and third and fourth nucleotides from the 5′ end) (see Table 1). PCR was performed essentially as provided in Example 1 using the forward (SEQ ID NO:20) and modified reverse (SEQ ID NO:54) primers that hybridized within the flanking homology sequences SEQ ID NO:50 and SEQ ID NO:51 to produce a double-stranded donor DNA molecule having an anti-CD38 DAR construct (SEQ ID NO:48) flanked by homology arms of 192 and 159 nts (SEQ ID NO:55 and SEQ ID NO:56). The resulting double stranded anti-CD38 DAR donor DNA fragment (SEQ ID NO:57) was three kilobases in size and incorporated the 2′-O-methyl and PS modifications of the reverse primer (SEQ ID NO:54) and the 5′ terminal phosphate of the forward primer (SEQ ID NO:20) into the donor DNA molecule, which was used to transfect activated PBMCs as a double-stranded molecule together with a Cas12a protein complexed with a crRNA (guide RNA) that included the target sequence (SEQ ID NO:52). The crRNA was an AltR® RNA purchased from IDT (Coralville, Iowa). Formation of the Cas12a and guide RNA RNP was performed essentially as described in Example 1 for the Cas9 RNP, except that no tracrRNA was used so there was no pre-hybridizaton of RNA species. Electroporation of the Cas12a RNP and the double-stranded donor DNA into T cells was also performed essentially according to Example 1. As controls, one T cell population was transformed with the Cas9 RNP but with no donor fragment and another T cell population was transformed with the Cas12a RNP but no donor fragment. In the absence of a donor fragment, the RNPs are predicted to disrupt the targeted gene but no expression construct is inserted. The transfected cells are therefore referred to as knockout (KO) controls.

Fourteen days after transfection, T cell populations transfected with the either the Cas9 RNP plus the donor DNA having homology arms for targeting the Cas9 target site (SEQ ID NO:49) or the Cas12a RNP and the donor DNA having homology arms for targeting the Cas12a target site (SEQ ID NO:57) were analyzed by flow cytometry alongside the knockout controls as described in Example 1 (FIGS. 15A to 15E). Only about 32% of the cell population that was transfected with a Cas9 RNP targeting the TRAC gene in the absence of a donor fragment and about 22% of the cell population that was transfected with a Cas12a RNP targeting the TRAC gene in the absence of a donor fragment (“TRAC KO”) expressed the TCR. By comparison, essentially all nonmodified activated T cells (activated T cells “ATC”), shown in FIG. 15A, expressed the TCR. As expected, none of the cells that did not receive donor DNA were positive for the anti-CD38 constructs (FIGS. 15B and 15C). On the other hand, significant percentages of cell populations transfected with an anti-CD38 DAR construct donor DNA in addition to a Cas9 or Cas12a RNP demonstrated expression of the DAR constructs: approximately 54% of the population transfected with the anti-CD38 DAR construct donor DNA along with a Cas9 RNP (FIG. 15D) expressed anti-CD38 DAR, as did approximately 77% of the population of cells transfected with the anti-CD38 DAR construct donor DNA along with a Cas12a RNP (FIG. 15E).

The insertion of the anti-CD38 DAR construct into the Cas9 target site of exon 1 of the TRAC gene, and insertion of the anti-CD38 DAR construct into the Cas12a target site of exon 1 of the TRAC gene were both confirmed by PCR performed on genomic DNA isolated from both transfected cell populations and sequencing of the junction fragments. For Cas9-mediated insertion, PCR of the 5′ homology arm region used SEQ ID NO:72 as the forward primer and SEQ ID NO:73 as the reverse primer. PCR of the 3′ homology arm region used SEQ ID NO:74 as the forward primer and SEQ ID NO:75 as the reverse primer. Sequencing of the resulting PCR fragments demonstrated that the anti-CD38 DAR construct had inserted in the targeted Cas9 target site. For Cas12a-mediated insertion, PCR of the 5′ homology arm region used SEQ ID NO:76 as the forward primer and SEQ ID NO:77 as the reverse primer. PCR of the 3′ homology arm region used SEQ ID NO:78 as the forward primer and SEQ ID NO:79 as the reverse primer. Sequencing of the resulting PCR fragments demonstrated that the anti-CD38 DAR construct had inserted in the targeted Cas12a target site.

The results of cytotoxicity assays with the transfected populations co-cultured with RPMI8226 cells is provided in FIG. 16 and demonstrated that T cells transfected with the DAR construct by using either a Cas9 or Cas12a system had the expected physiological behavior. Cells transfected with the Cas9 RNP plus anti-CD38 DAR construct donor DNA (SEQ ID NO:49 and the Cas12a RNP plus anti-CD38 DAR construct donor DNA (SEQ ID NO:57 both showed specific killing of RPMI88226 cells that was virtually identical and dramatically higher that of the control population having a knocked out TCR gene but not transfected with an anti-CD38 DAR construct donor DNA.

### Example 8. Analysis of Off-Target Mutations

To determine the frequency of off-site mutations resulting from transfection of culture with RNPs and donor fragments as provided herein, DNA isolated from cells from the transfection of PBMCs with the cas9 RNP targeting exon 1 of the TRAC gene and the double-stranded anti-CD38 DAR donor DNA having HAs of 171 and 161 bp synthesized with modified primers (SEQ ID NO:18 and SEQ ID NO:19) as provided in Example 7 was sequenced.

Genomic DNA was extracted from T cells with QIAamp® DNA Mini kit (QIAGEN 51104) according to the manufacturer's instructions. Briefly, a total of 5×10⁶ cells in 200 μL PBS were added to 20 μl QIAGEN Protease and 200 μl Buffer AL and incubated at 56° C. for 10 min. Genomic DNA were precipitated by ethanol and eluted from the mini-column. The concentration of genomic DNA was determined by Qubit 4 Flurometer using Qubit dsDNA HS Assay Kit (Thermofisher).

Whole genome sequencing of the DNA samples was performed by Novagene (Sacramento, Calif.). The results are summarized in FIG. 17 and Table 2. A total of 4 indels were detected. None of the detected indels were found to be in the coding regions of genes, with two of the off-site mutations found in intergenic regions, and two of the off-site mutations occurring in introns.

TABLE 2
Summary of Off-target mutations in anti-
CD38 DAR-T Cells generated with Cas9

| Type of Mutation | Number of events |
|---|---|
| CDS: | 0 |
| Frameshift deletion | 0 |
| Frameshift insertion | 0 |
| Nonframeshift deletion | 0 |
| Nonframeshift insertion | 0 |
| Stopgain | 0 |
| Stoploss | 0 |
| Unknown | 0 |
| Intronic | 1 |
| UTR3 | 0 |
| UTR5 | 0 |
| Splicing | 0 |
| ncRNA exonic | 0 |
| ncRNA intronic | 1 |
| ncRNA splicing | 0 |
| Upstream | 0 |
| Downstream | 0 |
| Intergenic | 2 |
| Total | 4 |

### Example 9. Targeted Insertion of an Anti-CD38 Dimeric Antibody Receptor (DAR) Construct into the TIM3 Locus with Cas12a

In further experiments, the anti-CD38 DAR construct was cloned between flanking sequences derived from the Tim-3 locus for simultaneously knocking out the Tim-3 gene, which may play a role in T cell exhaustion, and knocking in the anti-CD38 DAR using Cas112a. The anti-CD38 DAR construct (SEQ ID NO:48) was cloned between DNA sequences (5′ flanking sequence, SEQ ID NO:58; 3′ flanking sequence, SEQ ID NO:59) derived from the TIM3 gene locus and occurring on either side of a Cas112a target site (SEQ ID NO:60), which was immediately downstream of a Cas12a PAM sequence. The cloned DAR construct plus flanking sequences was used as a template for PCR reactions that used the forward primer 5′-p-TGGAATACAGAGCGGAGGTC (SEQ ID NO:60) and the reverse primer modified to include 2′-O-methyl groups on the first, second and third nucleotides from the 5′ end, and having PS bonds between the first and second, second and third, and third and fourth nucleotides from the 5′ end: mG*mC*mA*TGCAAATGTCCACTCAC (SEQ ID NO:61) to generate a donor DNA molecule (SEQ ID NO:62) that incorporated the modifications of the reverse primer (SEQ ID NO:61) into the 5′ end of one strand.

Transfection of T cells was done as performed in Example 1, except that in the Cas12a transfections the Cas12a protein was complexed with AltR crRNA and no tracr RNA was used. Donor fragment was electroporated along with the Cas12a RNP. As a control, a transfection was also done with the RNP in the absence of the donor DNA (TRAC knockout control).

The results of flow cytometry analysis of T cell populations transfected with the DAR construct targeted to the Tim-3 locus are provided in FIGS. 18A to 18B. Non-transformed activated T cells (ATC), included as a control, demonstrated expression of the Tim-3 gene by approximately 84% of the transfected cells while not expressing the anti-CD38 DAR construct. For knockout/knock-in cells, approximately 17% of the population expressed the CD38 DAR construct while not expressing the Tim-3 gene product.

### Example 10. Targeted Insertion of an Anti-CD38 Dimeric Antibody Receptor (DAR) Construct into the TRAC Locus with Second Site Knockout of the GM-CSF Gene Using Cas9 and Cas12a

The release of Granulocyte Marcrophage-Colony Stimulating Factor (GM-CSF) by T cells may contribute to cytokine release syndrome and neurotoxicity that may limit the therapeutic benefits of CAR-T therapy (Sterner et al. 2018 Blood 132:961). To provide a population of T cells that express an anti-CD38 DAR in place of the T cell receptor and in which the expression of GM-CSF is reduced, we attempted to 1) knock out the endogenous T cell receptor gene and knock in (at the TRAC locus) an anti-CD38 DAR construct, and also 2) knock out the GM-CSF gene, in the same cell population.

The anti-CD38 DAR construct described in Example 7 was used as a template for PCR to generate the donor fragment for TRAC knock-out and anti-CD38 DAR expression. This construct included the JeT promoter (SEQ ID NO:3) operably linked to nucleic acid sequences encoding the heavy chain polypeptide sequence with hinge, CD28 transmembrane domain, and 4-1BB and CD3ζ cytoplasmic domains, followed by the 2A peptide, and then the light chain polypeptide sequence, and also included an SV40 polyA addition sequence at the 3′end of the DAR-encoding sequence (anti-CD38 DAR-encoding assembly provided as SEQ ID NO:48) and was cloned between TRAC locus homology arms of 660 bp (SEQ ID NO:44) and 650 bp (SEQ ID NO:45) in plasmid vector pAAV-MCS. Donor fragment for use in transfection experiments was synthesized by PCR as described in Example 7 using a forward primer that included three PS bonds between the first and second, third and fourth, and fourth and fifth nucleotides and three 2′-O-methyl modifications at nucleotides 3, 4, and 5 when numbering from the 5′-terminus of the primer (SEQ ID NO:18), and a reverse primer that included a 5′ terminal phosphate (SEQ ID NO:19) (Table 1). The resulting PCR product (SEQ ID NO:49) included the homology arms of approximately 170 bp and 160 bp (SEQ ID NO:20 and SEQ ID NO:21) flanking the anti-CD38 DAR-encoding construct (SEQ ID NO:48) and had the primer modifications of SEQ ID NO:18 incorporated into the first strand and a 5′ terminal phosphate but no introduced chemical modifications added to the opposite, or second, strand.

The guide RNA for knocking out the TRAC locus consisted of two RNAs engineered for use with the S. pyogenes (Sp) cas9 protein: the crRNA for targeting exon 1 of the TRAC gene that included the target sequence of SEQ ID NO:1 and a tracrRNA, both of which were “AltR” RNAs engineered for use with Spcas9 and synthesized by IDT (Coralville, Iowa).

For knocking out the GM-CSF locus, a Cas12a guide specific for the human GM-CSF gene (target sequence TACAGAATGAAACAGTAGAAG, SEQ ID NO:80) was used. The crRNA designed for use with the Cas12a (Cpf1) nuclease was synthesized by IDT.

To modify the genome at two distinct sites, two RNPs were produced. The first RNP was a Cas9 RNP which was formed by incubating the Cas9 protein with a hybridized TRAC locus-guided crRNA (having target sequence SEQ ID NO:1) and a Cas9 tracrRNA. Hybridization of the Cas9 crRNA and tracrRNA and subsequent incubation of the Cas9 protein with the hybridized crRNA:tracrRNA targeting exon 1 of the TRAC gene was performed as provided in Example 1. The second RNP was a Cas12a RNP with was formed by incubating the Cas12a protein (that included an NLS at each of the N-terminal and C-terminal regions of the protein IDT) with a GM-CSF-targeting crRNA (having target sequence SEQ ID NO:80) in the same way (30 min incubation at 4° C.).

A single transfection of the T cells for knock-out of the TCR receptor gene with knock-in of the anti-CD38 DAR construct and knock-out of the GM-CSF gene was performed that used the Cas9 RNP assembled with the guide RNA targeting the TRAC gene and the Cas12a RNP targeting the GM-CSF gene, along with the donor fragment having HAs for insertion into the TRAC gene. Transfection was by electroporation using the same conditions as provided in Example 1.

The double-stranded chemically modified donor fragment having the sequence of SEQ ID NO:48 with the nucleotide modifications of primers SEQ ID NO:18 and SEQ ID NO:19 described in Example 7 was used to transfect cells along with the RNPs. The double-stranded donor fragment had HAs of 171 and 161 bp.

Three T cell populations were generated. In the first transfection activated T cells were transfected with the TRAC gene-targeting Cas9 RNP and the double stranded donor fragment encoding the anti-CD38 DAR and having TRAC HAs. A second transfection included the TRAC gene-targeting Cas9 RNP, the double stranded donor fragment encoding the anti-CD38 DAR and having TRAC HAs, and additionally, the Cas12a RNP targeting the GM-CSF gene. Finally, as a control, activated T cells were transfected with the TRAC-specific Cas9 RNP in the absence of a donor fragment, which should result in knockout of the targeted TRAC locus without DAR construct insertion. Electroporation was performed as detailed in Example 1, where in each case a single electroporation was performed that included, for the three populations respectively, 1) the TRAC-targeting RNP and anti-CD38 donor fragment; 2) the GM-CSF-targeting RNP in is addition to the TRAC-targeting RNP and the anti-CD38 donor fragment; and, 3) for the TRAC knockout only control, the TRAC-targeting RNP only.

Flow cytometry was performed essentially as described in Example 1 to evaluate the efficiency of introducing a different construct into the TRAC locus, where intracellular GM-CSF was detected using eBioscience™ Intracellular Fixation & Permeabilization Buffer Set, (ThermoFisher, 88-8824-00) and stained with PE-GM-CSF antibody [BioLegend, 502306]. CD3 (T cell receptor) was detected with anti-CD3-BV421 antibody SK7 (BioLegend), and anti-CD38 DAR expression was detected with PE conjugated anti-CD38-Fc protein (Chimerigen Laboratories, Allston, Mass.).

Where cultures were stimulated to induce GM-CSF expression, the cultures were treated for six hours with Cell Activation Cocktail (BioLegend, 423304) which contains phorbol-12-myristate 13-acetate (PMA, 40.5 μM), ionomycin (669.3 μM), and Brefeldin A (2.5 mg/ml) in DMSO.

FIGS. 19A to 19H shows in FIG. 19A and FIG. 19B that only approximately 2% of T cells transfected with the TRAC-targeting RNP and anti-CD38 DAR donor construct express GM-CSF when not stimulated with PMA and iononmycin, whereas stimulation of the transfected cells with these drugs results in approximately 53% of the cells producing GM-CSF. On the other hand, as seen in FIG. 19C, only approximately 29% of T cells transfected with the GM-CSF targeting RNP in addition to the TRAC-targeting RNP and anti-CD38 DAR donor construct produce GM-CSF on stimulation, evidence that simultaneous knock-out of a second gene occurs at a frequency estimated as 45% (52.78-29.07/52.78) in this case.

The same cell populations were analyzed for T cell receptor and anti-CD38 DAR expression in FIGS. 19E to 19G. FIG. 19D shows that when transfected with the TRAC targeting RNP in the absence of the anti-CD38 DAR donor fragment, nearly 80% (78.57%) of the cells did not express the T cell receptor and as expected, no expression of the anti-CD38 DAR construct was detected. In contrast, when cells were transfected with the TRAC targeting RNP along with the anti-CD38 DAR donor fragment, approximately 70% of the cell population demonstrated expression of the anti-CD38 DAR in the absence of expression of the native T cell receptor (FIG. 19E). Finally, addition of a second gene targeting RNP, the GM-CSF targeting RNP, did not drastically lower the TCR knockout/knockin rate, where approximately 50% of the cells transfected with both RNPs (anti-TRAC and anti-GM-CSF) plus the anti-CD38 DAR construct expressed the anti-CD38 DAR construct while failing to express the endogenous T cell receptor (FIG. 19F). In these cultures a high proportion (˜45%) of the population are calculated to be GM-CSF knockouts resulting from the inclusion of the anti-GM-CSF RNP to the transfection.

### Example 11. Targeted Insertion of an Anti-CD38 Dimeric Antibody Receptor (DAR) Construct into the TRAC Locus with Second Site Knockout of the GM-CSF Gene Using Cas12a

The double knock out of and concomitant knock in of the anti-CD38 DAR into the TRAC locus was also attempted using two Cas12a RNPs, each of which included a different guide RNA (crRNA).

The anti-CD38 DAR donor fragment was produced as described in Examples 7 and 10, above, using a forward primer that included three PS bonds between the first and second, third and fourth, and fourth and fifth nucleotides and three 2′-O-methyl modifications at nucleotides 3, 4, and 5 when numbering from the 5′-terminus of the primer (SEQ ID NO:18), and a reverse primer that included a 5′ terminal phosphate (SEQ ID NO:19) (Table 1). The resulting PCR product (SEQ ID NO:49) included the homology arms of approximately 170 bp and 160 bp (SEQ ID NO:20 and SEQ ID NO:21) flanking the anti-CD38 DAR-encoding construct (SEQ ID NO:48) and had the primer modifications of SEQ ID NO:18 incorporated into the first strand and a 5′ terminal phosphate but no introduced chemical modifications added to the opposite, or second, strand.

The guide RNA for knocking out the TRAC locus was a crRNA engineered for Cas12a (Cpf1) for targeting exon 1 of the TRAC gene and included the target sequence of SEQ ID NO:26. The guide RNA for knocking out the TRAC gene was a crRNA engineered for Cas12a (Cpf1) for targeting exon 1 of the TRAC gene and included the target sequence of SEQ ID NO:52. The guide RNA for knocking out the GM-CSF gene was a crRNA engineered for Cas12a (Cpf1) that included the target sequence of SEQ ID NO:80. Both Cas12a crRNAs were synthesized by IDT (Coralville, Iowa).

Two RNPs were produced with a Cas12a protein that included an NLS at each of the N-terminal and C-terminal regions of the protein (IDT). The first RNP was formed by incubating the Cas12a protein with the crRNA targeting the TRAC locus and having the target sequence of SEQ ID NO:52 way (30 min incubation at 4° C.). The second RNP was formed by incubating the Cas12a protein with a GM-CSF-targeting crRNA (having target sequence SEQ ID NO:80) in the same way.

A single transfection of the T cells for knock-out of the TCR receptor gene with knock-in of the anti-CD38 DAR construct and knock-out of the GM-CSF gene was performed that used the two assembled Cas12a RNPs along with the donor fragment having HAs for insertion into the TRAC gene. Transfection was by electroporation using the same conditions as provided in Example 1.

The double-stranded chemically modified donor fragment having the sequence of SEQ ID NO:48 with the nucleotide modifications of primers SEQ ID NO:18 and SEQ ID NO:19 described in Example 7 was used to transfect cells along with the RNPs. The double-stranded donor fragment had HAs of 171 and 161 bp.

Flow cytometry was performed essentially as described in Example 10 to evaluate the efficiency of introducing a different construct into the TRAC locus, where intracellular GM-CSF was detected using eBioscience™ Intracellular Fixation & Permeabilization Buffer Set, (ThermoFisher, 88-8824-00) and stained with PE-GM-CSF antibody [BioLegend, 502306]. CD3 (T cell receptor) was detected with anti-CD3-BV421 antibody SK7 (BioLegend), and anti-CD38 DAR expression was detected with PE conjugated anti-CD38-Fc protein (Chimerigen Laboratories, Allston, Mass.).

FIGS. 19A and 19B show that only approximately 2% of T cells transfected with the TRAC-targeting RNP and anti-CD38 DAR donor construct express GM-CSF when not stimulated whereas stimulation of the transfected cells with these drugs results in approximately 53% of the cells producing GM-CSF. On the other hand, as seen in FIG. 19D, only approximately 15% of T cells transfected with the GM-CSF targeting RNP in addition to the TRAC-targeting RNP and anti-CD38 DAR donor construct produce GM-CSF on stimulation, evidence that simultaneous knock-out of a second gene occurs at a frequency estimated as 72% (52.78-14.9/52.78) in this case.

The same cell populations were analyzed for T cell receptor and anti-CD38 DAR expression in FIG. 19H. FIG. 19E shows that when transfected with the TRAC targeting RNP in the absence of the anti-CD38 DAR donor fragment, nearly 80% (78.57%) of the cells did not express the T cell receptor and as expected, no expression of the anti-CD38 DAR construct was detected. In contrast, when cells were transfected with the TRAC targeting RNP along with the anti-CD38 DAR donor fragment, approximately 70% of the cell population demonstrated expression of the anti-CD38 DAR in the absence of expression of the native T cell receptor (FIG. 19F). Finally, addition of a second gene targeting RNP, the GM-CSF targeting RNP, did not drastically lower the TCR knockout/knockin rate, where approximately 60% of the cells transfected with both RNPs (anti-TRAC and anti-GM-CSF) plus the anti-CD38 DAR construct expressed the anti-CD38 DAR construct while failing to express the endogenous T cell receptor (FIG. 19H). In these cultures a high proportion (˜49%) of the population are calculated to be GM-CSF knockouts resulting from the inclusion of the anti-GM-CSF RNP to the transfection.

### Example 12. Targeted Insertion of an Anti-CD20 Dimeric Antibody Receptor (DAR) Construct into the TRAC Gene Using Cas12a

The T cell receptor alpha constant (TRAC) gene was also targeted with an anti-CD20 DAR construct as the donor DNA. The anti-CD20 DAR construct (SEQ ID NO:81) included a nucleic acid sequence encoding two polypeptides linked by a “self-cleaving” 2A sequence that was used to generate two polypeptides from a single open reading frame. The first encoded polypeptide was a heavy chain polypeptide that included a heavy chain variable (VH) and the first heavy chain constant region (CH1), a hinge region, a transmembrane domain of CD28, and a cytoplasmic domain of 4-1BB and the third ITAM of CD3ζ. This was followed by the Thosea asigna virus T2A peptide-encoding sequence (SEQ ID NO:46) and then by the sequence encoding the second polypeptide, where the second polypeptide included, proceeding from the N-terminus to the C-terminus, an immunoglobulin light chain variable (VL) plus constant region (kappa). The nucleic acid sequences encoding the heavy chain polypeptide sequence, 2A peptide, and light chain sequence were operably linked to the JeT promoter (SEQ ID NO:3) at the 5′ end of the DAR-encoding sequence and an SV40 polyA addition sequence (SEQ ID NO:47) at the 3′end of the DAR-encoding sequence. The entire anti-CD20 DAR construct (JeT promoter, heavy chain-encoding sequences with hinge, transmembrane of CD28, and cytoplasmic domains of 4-1BB and CD3ζ followed by T2A, light chain, and SV40 sequence (SEQ ID NO:48)), was cloned between homology arms of 645 bp (SEQ ID NO:50) and 600 bp (SEQ ID NO:51) in a pAAV vector. The homology arms (HAs) were sequences of the TRAC exon 1 locus on either side of the target sequence (SEQ ID NO:52) in exon 1 of the TRAC gene.

Donor fragment for use in transfection experiments was synthesized by PCR as described in Example 1 using the pAAV anti-CD20 DAR vector construct that included flanking TRAC HAs. The primers used were SEQ ID NO:82 (forward primer) which was 5′ phosphorylated and SEQ ID NO:54 (reverse primer) which included 2′-O-methyl modifications on the three 5′-most nucleotides of the primer and phosphorothiate bonds between the first and second, second and third, and third and fourth nucleotides from the 5′ end of the primer (Table 1). These primers hybridized within the 645 bp and 600 bp homology arms in the vector construct to generate a fragment with homology arms of 192 bp and 159 bp flanking the DAR construct. The resulting PCR product, a double stranded anti-CD20 DAR donor DNA fragment (SEQ ID NO:83) was 2.8 kb in size and including the 2.457 kb anti-CD20 DAR construct (SEQ ID NO:81), a 192 bp homology arm (SEQ ID NO:55), and a 159 bp homology arm (SEQ ID NO:56) flanking the anti-CD20 DAR-encoding construct (SEQ ID NO:81) and incorporated the 2′-O-methyl and PS modifications of the reverse primer (SEQ ID NO:54) and the 5′ terminal phosphate of the forward primer (SEQ ID NO:82) into the donor DNA molecule. The primer modifications of SEQ ID NO:54 incorporated into the first strand but no chemical modifications were introduced into the opposite, or second, strand, which include the 5′ terminal phosphate of the primer. The donor molecule was used to transfect activated T cells as a double-stranded molecule together with a Cas12a protein complexed with a crRNA (guide RNA) that included the target sequence (SEQ ID NO:52).

For RNA guide-directed targeting of the TCR alpha (TRAC) gene, the crRNA (ALT-R® CRISPR-Cas12a crRNA) was purchased from IDT (Coralville, Iowa), where the crRNA was designed to include the target sequence (SEQ ID NO:52) that occurs directly downstream of a Cas12a PAM sequence (TIA) in first exon of the TRAC gene.

Formation of the Cas12a and guide RNA RNP was performed essentially as described in Example 7. Electroporation of the Cas12a RNP and the double-stranded donor DNA into T cells was also performed essentially according to Example 7. As a control, one T cell population was transfected with the Cas12a RNP but no donor fragment, referred to as the TRAC knockout (KO) control. After transfection, T cells were transferred to complete cell culture medium for expansion.

After ten days, the cultures were analyzed by flow cytometry alongside the TRAC knockout control population as described in Example 1 except CD20 DAR expression was detected by Anti-Rituximab Antibodies (Acro) (FIGS. 20A and 20B). Only about 16.6% of the cell population that was transfected with a Cas12a RNP targeting the TRAC gene in the absence of a donor fragment (“TRAC KO”) expressed the TCR. An even smaller percentage, about 3.5%, of the cell population that was transfected with a Cas12a RNP targeting the TRAC gene along with a CD20 DAR donor fragment (“CD20 DAR-T”) expressed the TCR. As expected, none of the cells that did not receive donor DNA were positive for the anti-CD20 constructs (FIG. 20A). On the other hand, 28.7% of the population transfected with the anti-CD20 DAR construct donor DNA along with a Cas12a RNP (FIG. 203) expressed anti-CD20 DAR while not expressing the TCR.

The cells were tested in cytotoxicity assays performed essentially as in Example 1, except that the anti-CD20 DAR cells were incubated with CD20+ Daudi cells as targets for two days. FIG. 21 shows that the level of killing was very high, approaching 90% for effector:target ratios ranging from 0.625:1 to 5:1, and even at the lowest effector:target ratio of 0.16:1, was approximately 70%. FIGS. 22A to 22B demonstrates that the anti-CD20 CAR-T cells secreted a high level of interferon gamma (IFNγ) and GM-CSF when stimulated by CD20+ Daudi cells. Secretion of cytokines by anti-CD19 CAR-T cells (see Example 4) is also shown.

### Example 13. Targeted Insertion of an Anti-CEA CAR Construct into the TRAC and CD7 Genes Using Cas12a

An anti-CEA CAR construct was also inserted into the TRAC gene using Cas12a. The anti-CEA CAR construct (SEQ ID NO:84) that included the JeT promoter (SEQ ID NO:3) at the 5′ end of the CAR-encoding sequence and an SV40 polyA addition sequence (SEQ ID NO:47) at the 3′end of the CAR-encoding sequence was cloned between homology arms of 645 bp (SEQ ID NO:50) and 600 bp (SEQ ID NO:51) in a pAAV vector. The homology arms were sequences of the TRAC exon 1 locus on either side of the target sequence (SEQ ID NO:52) in exon 1 of the TRAC gene.

Donor fragment for use in transfection experiments was synthesized by PCR as described in Example 1. The primers used were SEQ ID NO:82 (forward primer) which was 5′ phosphorylated and SEQ ID NO:54 (reverse primer) which included 2′-O-methyl modifications on the three 5′-most nucleotides of the primer and phosphorothiate bonds between the first and second, second and third, and third and fourth nucleotides from the 5′ end of the primer (Table 1). These primers hybridized within the 645 bp and 600 bp homology arms in the vector construct, to generate a construct with homology arms of 192 bp and 159 bp flanking the DAR construct. The resulting PCR product, a double stranded anti-CEA CAR donor DNA fragment (SEQ ID NO:85) was 2.4 kb in size and including the 2.077 kb anti-CEA CAR construct (SEQ ID NO:84), a 192 bp homology arm (SEQ ID NO:55), and a 159 bp homology arm (SEQ ID NO:56) flanking the anti-CEA CAR construct and incorporated the 2′-O-methyl and PS modifications of the reverse primer (SEQ ID NO:54) and the 5′ terminal phosphate of the forward primer (SEQ ID NO:82) into the donor DNA molecule. The primer modifications of SEQ ID NO:54 were incorporated into the first strand of the double-stranded donor DNA but no chemical modifications were introduced into the opposite, or second, strand, which included the 5′ terminal phosphate of the primer. The donor molecule was used to transfect activated T cells as a double-stranded molecule together with a Cas12a protein complexed with a crRNA (guide RNA) that included the target sequence (SEQ ID NO:52).

For RNA guide-directed targeting of the TCR alpha (TRAC) gene, the crRNA (ALT-R® CRISPR-Cas12a crRNA (ALT-R® CRISPR-Cas12a crRNA) was purchased from IDT (Coralville, Iowa), where the crRNA was designed to include the target sequence (SEQ ID NO:52) that occurs directly downstream of a Cas12a PAM sequence (TTTA) in first exon of the TRAC gene.

Formation of the Cas12a and guide RNA RNP and electroporation of the Cas12a RNP and the double-stranded donor DNA into T cells was also performed essentially according to Example 7. As a control, one T cell population was transformed with the Cas12a RNP but no donor fragment, referred to as the TRAC knockout (KO) control.

In a separate experiment, the same anti-CEA CAR construct was inserted into the CD7 gene using Cas12a. The anti-CEA CAR construct (SEQ ID NO:84) in this case was cloned between homology arms comprising sequences surrounding the CD7 target site (SEQ ID NO:86) in a pAAV vector.

To select the target site in the CD7 gene, two potential sites upstream of a Cas12a PAM sequence were investigated, each of which received a high score for achieving knockout using an online guide analysis tool for Cpf1 (Cas12a). The first target site (SEQ ID NO:86) was used to design the RNA guide “crRNA-1”; this target sequence had only one site match in the human genome outside of the targeted CD7 locus, and the additional site in the genome was not within an exon. The second target sequence (SEQ ID NO:87) identified in the CD7 gene as being upstream of a Cas12a PAM site was used to design guide RNA “crRNA-2”. This site had 103 matching sequences in the human genome, of which five occurred in exons. Interestingly, knockout experiments (no donor included in the electroporation) using the two guides and flow cytometry analysis showed that using crRNA-2 as the guide resulted in about 80% of the transfected population losing CD7 expression, whereas crRNA-1 as a guide resulted in approximately 96% of the transfected population losing CD7 expression. The crRNA-1 guide RNA directed to the target sequence (SEQ ID NO:86) was therefore selected for knockout/knockin in the CD7 gene.

Donor fragment for insertion into the CD7 locus was synthesized by PCR as described in Example 1. The primers used were SEQ ID NO:88 (forward primer) which included 2′-O-methyl modifications on the first, third, and fourth nucleotides from the 5′ end of the primer and phosphorothiate bonds between the first and second, second and third, and third and fourth nucleotides from the 5′end, and SEQ ID NO:89 (reverse primer) which was 5′ phosphorylated (Table 1). These primers hybridized within homology arms in a vector construct that included the anti-CEA CAR construct (SEQ ID NO:84) flanked by extended homology arms that flanked the CD7 target site (SEQ ID NO:86) to generate a donor fragment with homology arms of 212 bp (SEQ ID NO:90) and 170 bp (SEQ ID NO:91) flanking the CAR construct. The resulting PCR product, a double stranded anti-CEA CAR donor DNA fragment (SEQ ID NO:92) was 2.46 kb in size, including the 2.077 kb anti-CEA CAR construct (SEQ ID NO:84), a 212 bp homology arm (SEQ ID NO:90), and a 170 bp homology arm (SEQ ID NO:91) and incorporated the 2′-O-methyl and PS modifications of the forward primer (SEQ ID NO:88) and the 5′ terminal phosphate of the reverse primer (SEQ ID NO:89) into the donor DNA molecule. The primer modifications of SEQ ID NO:88 were incorporated into the first strand but no chemical modifications were introduced into the opposite, or second, strand, which include the 5′ terminal phosphate of the primer. The donor DNA was used to transfect activated T cells as a double-stranded molecule together with a Cas12a protein complexed with a crRNA (guide RNA) that included the target sequence (SEQ ID NO:87).

For RNA guide-directed targeting of the CD7 gene, the crRNA (ALT-R® CRISPR-Cas12a crRNA) was purchased from IDT (Coralville, Iowa), where the crRNA was designed to include the target sequence (SEQ ID NO:86) that occurs directly downstream of a Cas12a PAM sequence (TTTA) in first exon of the TRAC gene.

Formation of the Cas12a and guide RNA RNP and electroporation of the Cas12a RNP and the double-stranded donor DNA into T cells was also performed essentially according to Example 7. As a control, one T cell population was transformed with the Cas12a RNP but no donor fragment, referred to as the TRAC knockout (KO) control.

The transfected cultures were analyzed by flow cytometry alongside the TRAC knockout control population as described above (FIGS. 23A to 23D). FIG. 23A shows that there was no detected expression of the CEA CAR in cells that were not transfected with donor DNA, although 89% of the population that was electroporated with a TRAC guide-RNP did lose expression of the TRAC gene (knockouts). When the donor was included in the electroporation, however, approximately 28.7% of the population failed to express the TCR while expressing the anti-CEA CAR (FIG. 23B). Targeting the CD7 locus with an anti-CEA CAR donor and Cas12a RNP was even more efficient: approximately 38% of the population were knockin/knockout cells (expression of the anti-CEA CAR in the absence of CD7 expression) (FIG. 23D). FIG. 23C provides a basis for comparison of CD7 expression in cells in which the CD7 locus was not targeted (the cells were transfected with an RNP targeting the TRAC locus). In this population about 65% of the cell population expressed CD7 as compared with only about 8% of the population in which the CD7 locus was targeted with an RNP (FIG. 23D).

The cells were tested in cytotoxicity assays performed essentially as in Example 1 using CEA positive LS174T cells as targets. FIG. 24 shows that, as expected, TRAC knockout cells that did not express the anti-CEA CAR did not kill the targets. Cas12a-mediated knockin of the anti-CEA CAR at the TRAC locus on other hand resulted in cytotoxicity that was dependent on the effector: target ratio, reaching levels of 60% killing at effector:target ratios greater than 2.5:1. Interestingly, anti-CEA CAR knockins at the CD7 locus were much more effective at killing targets than knockins at the TRAC locus, especially at low target:effector ratios, demonstrating approximately 80% killing even at the lowest target to effector ratio of 0.625:1.

FIG. 25 shows the both the Cas12a-mediated anti-CEA CAR knockin/CD7 knockout and the anti-CEA CAR knockin/TRAC knockout T cells secreted interferon gamma, with the CD7 knockout/anti-CEA CAR knockin T cells secreting somewhat less interferon gamma than the TRAC knockout/anti-CEA CAR knockin T cells.

1. A method for genetically modifying a primary human T cell at two different genetic loci, comprising introducing into a primary human T cell:
a first ribonucleoprotein (RNP) comprising a first RNA-guided endonuclease and a first guide RNA;
a second RNP comprising a second RNA-guided endonuclease and a second guide RNA; and
a donor DNA molecule comprising at least two nucleic acid modifications;
wherein the first guide RNA comprises a target sequence designed to hybridize with a first target site at a first genetic locus in the target DNA and the donor DNA is inserted into the target DNA molecule at the first target site;
further wherein the second guide RNA comprises a second target sequence designed to hybridize with a second target site at a second genetic locus in the target DNA resulting in a mutation at the second target site.

2. A method according to claim 1, wherein the at least two nucleic acid modifications are on a single strand of the donor DNA molecule.

3. A method according to claim 1 or 2, wherein one or more nucleic acid modifications are a modification of one or more nucleotides or nucleotide linkages within nucleotides of the 5′ end of the modified strand of the donor DNA molecule.

4. A method according to claim 1, wherein one or more nucleic acid modifications is a backbone modification.

5. A method according to claim 4, wherein one or more nucleic acid modifications is a phosphorothioate modification or a phosphoramidite modification, or a combination thereof.

6. A method according to claim 1, wherein one or more nucleic acid modifications is a modification or substitution of a nucleobase.

7. A method according to claim 1, wherein one or more nucleic acid modifications is a modification or substitution of a sugar.

8. A method according to claim 7, wherein one or more nucleic acid modifications is a 2′-O-methyl group modification of deoxyribose.

9. A method according to claim 1 or 2, wherein the donor DNA molecule is a double stranded DNA molecule.

10. A method according to claim 9, wherein the donor DNA molecule has a 5′ terminal phosphate on the strand opposite to the modified strand.

11. A method according to claim 10, wherein the donor molecule has between one and three phosphorothiorate modifications on the backbone within ten nucleotides of the 5′ terminus of the modified strand of the donor molecule and between one and three 2′-O-methyl nucleotide modifications within ten nucleotides of the 5′ terminus of the modified strand of the donor molecule.

12. A method according to claim 11, wherein the donor molecule has between one and three phosphorothiorate modifications on the backbone within five nucleotides of the 5′ terminus of the modified strand of the donor molecule and between one and three 2′-O-methyl nucleotide modifications within five nucleotides of the 5′ terminus of the modified strand of the donor molecule.

13. A method according to claim 1, wherein the donor DNA molecule includes homology arms flanking a sequence for integration into the genome.

14. A method according to claim 13, wherein at least one of the homology arms is from 50 to 2000 nucleotides in length.

15. A method according to claim 13, wherein at least one of the homology arms is from 140 to 660 nucleotides in length.

16. A method according to claim 13, wherein at least one of the homology arms is from 140 to 250 nucleotides in length.

17. The method of claim 13, wherein the donor DNA molecule comprises a modified strand and an opposite strand, wherein the modified strand comprises two or more nucleic acid modifications, and the opposite strand comprises a terminal phosphate.

18. The method of claim 1, wherein the donor DNA is from about 500 to about 5000 bp in length.

19. The method of claim 18, wherein the donor DNA is from about 500 to about 3500 bp in length.

20. The method of claim 1, wherein the donor DNA comprises a chimeric antigen receptor (CAR) or dimeric antigen receptor (DAR) construct.

21. A method according to claim 1, wherein the first and/or the second RNA-guided endonuclease is Cas9.

22. A method according to claim 21, wherein the first and/or the second RNA-guided endonuclease is Cas12a.

23. A method according to claim 22, wherein the first and the second RNA-guided endonuclease are Cas12a.

24. A method according to claim 21, wherein the first RNA-guided endonuclease is Cas12a and the second RNA-guided endonuclease is Cas9 or wherein the first RNA-guided endonuclease is Cas9 and the second RNA-guided endonuclease is Cas12a.

25. A method according to claim 1, wherein the first RNP and the donor DNA are introduced at the same time.

26. A method according to claim 25, wherein the first RNP, the donor DNA, and the second RNP are introduced into the cell at the same time.

27. A method according to claim 1, wherein the first RNP and the donor DNA are introduced into the cell at the same time, and the second RNP is introduced into the cell at a different time.

28. The method of claim 27, wherein the RNP is introduced into the cell by electroporation or liposome transfer.

29. An engineered primary T cell comprising:
a non-native genetic construct integrated into the genome at a first genetic locus comprising a first target site of an RNA-guided nuclease and a mutation at a second genetic locus comprising a second target site of an RNA-guided nuclease, w % herein the engineered primary T cell is produced by the method of any of claims 1-28.

30. A population of primary human T cells transfected with a genetic construct, wherein the cell population comprises cells having a non-native genetic construct integrated into the genome at a first genetic locus, and further have a mutation in a gene at a second genetic locus, wherein at least 25% of the cells of the population express the genetic construct and exhibit reduced expression of the gene at the second genetic locus.

31. A population of primary human T cells according to claim 30, wherein the first RNA-guided endonuclease target site and the second RNA-guided endonuclease site are Cas12a target sites.

32. A population of human T cells according to claim 30, wherein the first RNA-guided endonuclease target site is a Cas12a target site and the second RNA-guided endonuclease site is a Cas9 target site.

33. A population of human T cells according to claim 30, wherein the first RNA-guided endonuclease target site is a Cas9 target site and the second RNA-guided endonuclease site is a Cas12a target site.

34. A method for site-specific integration of a donor DNA into a target DNA molecule, comprising:
introducing into a cell:
an RNP comprising a Cas12a endonuclease and an engineered guide RNA; and
a donor DNA molecule comprising at least two nucleic acid modifications;
wherein the guide RNA comprises a target sequence designed to hybridize with a target site in the target DNA and the donor DNA is inserted into the target DNA molecule at the target site.

35. A method according to claim 34, wherein the at least two nucleic acid modifications are on a single strand of the donor DNA molecule.

36. A method according to claim 34 or 35, wherein one or more nucleic acid modifications are a modification of one or more nucleotides or nucleotide linkages within nucleotides of the 5′ end of the modified strand of the donor DNA molecule.

37. A method according to claim 34, wherein one or more nucleic acid modifications is a backbone modification.

38. A method according to claim 37, wherein one or more nucleic acid modifications is a phosphorothioate modification or a phosphoramidite modification, or a combination thereof.

39. A method according to claim 33, wherein one or more nucleic acid modifications is a modification or substitution of a nucleobase.

40. A method according to claim 33, wherein one or more nucleic acid modifications is a modification or substitution of a sugar.

41. A method according to claim 40, wherein one or more nucleic acid modifications is a 2′-O-methyl group modification of deoxyribose.

42. A method according to claim 34, wherein the donor DNA molecule is a double stranded DNA molecule.

43. A method according to claim 42, wherein the donor DNA molecule has a 5′ terminal phosphate on the strand opposite to the modified strand.

44. A method according to claim 43, wherein the donor molecule has between one and three phosphorothiorate modifications on the backbone within ten nucleotides of the 5′ terminus of the modified strand of the donor molecule and between one and three 2′-O-methyl nucleotide modifications within ten nucleotides of the 5′ terminus of the modified strand of the donor molecule.

45. A method according to claim 44, wherein the donor molecule has between one and three phosphorothiorate modifications on the backbone within five nucleotides of the 5′ terminus of the modified strand of the donor molecule and between one and three 2′-O-methyl nucleotide modifications within five nucleotides of the 5′ terminus of the modified strand of the donor molecule.

46. A method according to claim 34, wherein the donor DNA molecule includes homology arms flanking a sequence for integration into the genome.

47. A method according to claim 46, wherein at least one of the homology arms is from 50 to 2000 nucleotides in length.

48. A method according to claim 47, wherein at least one of the homology arms is from 140 to 660 nucleotides in length.

49. A method according to claim 48, wherein at least one of the homology arms is from 140 to 250 nucleotides in length.

50. The method of claim 34, wherein the donor DNA molecule comprises a modified strand and an opposite strand, wherein the modified strand comprises two or more nucleic acid modifications, and the opposite strand comprises a terminal phosphate.

51. The method of claim 34, wherein the donor DNA is from about 500 to about 5000 bp in length.

52. The method of claim 51, wherein the donor DNA is from about 500 to about 3500 bp in length.

53. The method of claim 34, wherein the donor DNA comprises a chimeric antigen receptor (CAR) or dimeric antigen receptor (DAR) construct.

54. A method according to claim 34, wherein the guide RNA is a crRNA.

55. A method according to claim 54, further comprising introducing a tracr RNA into the cell.

56. The method of claim 34, wherein the RNP is introduced into the cell by electroporation or liposome transfer.

57. The method of claim 34, wherein the donor DNA and the RNP are introduced into the cell simultaneously or separately.

58. A system for targeted integration of a donor DNA into a target locus, comprising:
a Cas12a endonuclease;
a guide RNA; and
a double-stranded donor DNA molecule, wherein the donor DNA molecule includes one or more phosphorothioate bonds on a single modified strand of the double stranded DNA molecule within ten nucleotides of the 5′ terminus of the modified strand of the double stranded DNA molecule.

59. The system of claim 58, wherein the donor DNA molecule further comprises at least one modification of a sugar moiety or nucleobase of the modified strand within ten nucleotides of the 5′ terminus of the modified strand of the double stranded DNA molecule.

60. The system of claim 58, wherein the donor DNA has homology arms flanking a sequence of interest for integration into the genome.

61. The system of claim 58, wherein the one or more phosphorothioate bonds on the single modified strand of the double stranded DNA molecule is within five nucleotides of the 5′ terminus of the modified strand of the double stranded DNA molecule.

62. The system of claim 61, wherein the at least one modification of a sugar moiety or nucleobase of the modified strand is within five nucleotides of the 5′ terminus of the modified strand of the double stranded DNA molecule.

63. The system of claim 61, wherein the at least one modification of a sugar moiety comprises a 2′-O methylation.

64. The system of claim 60, wherein the sequence of interest comprises an expression cassette.

65. The system of claim 64, wherein the expression cassette comprises a construct comprising one or more antibody or receptor domains.

66. A system according to claim 60, wherein at least one of the homology arms is from 50 to 2000 nucleotides in length.

67. A system according to claim 6, wherein at least one of the homology arms is from 140 to 660 nucleotides in length.

68. A system according to claim 6, wherein at least one of the homology arms is from 140 to 250 nucleotides in length.

69. The system of claim 61, wherein the donor DNA molecule comprises a modified strand and an opposite strand, wherein the modified strand comprises two or more nucleic acid modifications, and the opposite strand comprises a terminal phosphate.

70. The system of claim 58, wherein the donor DNA is from about 500 to about 5000 bp in length.

71. The system of claim 58, wherein the donor DNA is from about 500 to about 3500 bp in length.

72. The system of claim 64, wherein the donor DNA comprises a chimeric antigen receptor (CAR) or dimeric antigen receptor (DAR) construct.

73. The system of claim 58, wherein the guide RNA is a crRNA.

74. The system of claim 58, wherein the guide RNA comprises one or more phosphorothioate (PS) oligonucleotides.

75. The system of claim 58, comprising a ribonucleoprotein complex comprising the cas12a endonuclease and the guide RNA.

76. A primary human T cell having a CAR or DAR construct inserted into the CD7 gene.

77. A primary human T cell according to claim 76, wherein the CAR or DAR construct is a CEA CAR or DAR construct.

78. A population of T cells according to claim 76.


SEQUENCE LISTING (91 sequences)

SEQ ID NO: 1 - DNA, 19 bp, Homo sapiens
  misc_feature (1)..(19): Target sequence in TRAC gene (exon 1)
cagggttctggatatctgt

SEQ ID NO: 2 - DNA, 2119 bp, Artificial Sequence
  Synthetic anti-CD38 CAR Construct for insertion into TRAC locus
  misc_feature: includes the JeT promoter followed by a DNA sequence encoding CD8a leader peptide followed by anti-CD38 CAR (single chain variable fragment (scFv) specific for human CD38)
  misc_feature: followed by CD28 hinge-transmembrane- intracellular regions and CD3 zeta intracellular domain
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 3 - DNA, 195 bp, Artificial Sequence
  Synthetic Jet promoter
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 4 - DNA, 3429 bp, Artificial Sequence
  Synthetic anti-CD38A2 CAR cassette with homology arms
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 5 - DNA, 20 bp, Artificial Sequence
  Synthetic primer for sequencing clones with anti-CD38 CAR-TRAC homology arms insert in pAAV-MCS vector
cttaggctgggcattagcag

SEQ ID NO: 6 - DNA, 20 bp, Artificial Sequence
  Synthetic primer for sequencing clones with anti-CD38 CAR-TRAC homology arms insert in pAAV-MCS vector
catggaatggtcatgggtct

SEQ ID NO: 7 - DNA, 20 bp, Artificial Sequence
  Synthetic primer for sequencing clones with anti-CD38 CAR-TRAC homology arms insert in pAAV-MCS vector
ggctacgtattcggttcagg

SEQ ID NO: 8 - DNA, 20 bp, Artificial Sequence
  Synthetic Forward primer for generating donor DNA PCR fragment from pAAV anti-CD38 CAR-TRAC construct (660 & 650 HAs)
  modified_base (1)..(2): Phosphorothioate linkage
  modified_base (2)..(3): Phosphorothioate linkage
  modified_base (2)..(3): 2′-O-methylated
  modified_base (3)..(4): Phosphorothioate linkage
  modified_base (4)..(4): 2′-O-methylated
tggagctagggcaccatatt

SEQ ID NO: 9 - DNA, 20 bp, Artificial Sequence
  Synthetic Reverse primer for generating donor DNA PCR fragment from pAAV anti-CD38 CAR-TRAC construct (660 & 650 HAs), the 5′-most nucleoside (C) has a 5′ phosphate
  misc_feature: 5′ phosphate
caacttggagaaggggctta

SEQ ID NO: 10 - DNA, 20 bp, Artificial Sequence
  Synthetic PCR forward primer with homology to TRAC locus for verifying site-specific insertion of anti-CD38 CAR (upstream junction)
cctgctttctgagggtgaag

SEQ ID NO: 11 - DNA, 20 bp, Artificial Sequence
  Synthetic PCR reverse primer with homology to CAR construct for verifying site-specific insertion of anti-CD38 CAR (upstream junction)
ctttcgaccaactggacctg

SEQ ID NO: 12 - DNA, 20 bp, Artificial Sequence
  Synthetic PCR forward primer with homology to CAR locus for verifying site-specific insertion of anti-CD38 CAR (downstream junction)
cgttctgggtactcgtggtt

SEQ ID NO: 13 - DNA, 20 bp, Artificial Sequence
  Synthetic PCR reverse primer with homology to TRAC locus (downstream junction)
gagagcccttccctgacttt

SEQ ID NO: 14 - DNA, 20 bp, Artificial Sequence
  Synthetic Forward primer for generating donor fragment with 300 nt HAs
  modified_base (1)..(2): Phosphorothioate linkage
  modified_base (2)..(3): Phosphorothioate linkage
  modified_base (2)..(3): 2′-O-methylated
  modified_base (3)..(4): Phosphorothioate linkage
  modified_base (5)..(5): 2′-O-methylated
ccatgcctgcctttactctg

SEQ ID NO: 15 - DNA, 20 bp, Artificial Sequence
  Synthetic Reverse primer for generating donor fragment with 300 nt HAs, the 5′-most nucleoside (T) has a 5′ phosphate
  misc_feature: 5′ phosphate
tcctgaagcaaggaaacagc

SEQ ID NO: 16 - DNA, 375 bp, Homo sapiens
  misc_feature (1)..(375): 5′ homology arm, exon 1 TRAC gene
ccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 17 - DNA, 321 bp, Homo sapiens
  misc_feature (1)..(321): 3′ homology arm, exon 1 TRAC gene
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcagga

SEQ ID NO: 18 - DNA, 20 bp, Artificial Sequence
  Synthetic Forward primer for generating donor fragment with 150 nt HAs
  modified_base (1)..(2): Phosphorothioate linkage
  modified_base (3)..(4): Phosphorothioate linkage
  modified_base (3)..(5): 2′-O-methylated
  modified_base (4)..(5): Phosphorothioate linkage
atcacgagcagctggtttct

SEQ ID NO: 19 - DNA, 24 bp, Artificial Sequence
  Synthetic Reverse primer for generating donor fragment with 150 nt HAs, the 5′-most nucleoside (G) has a 5′ phosphate
  misc_feature: 5′ phosphate
gacctcatgtctagcacagttttg

SEQ ID NO: 20 - DNA, 171 bp, Homo sapiens
  misc_feature (1)..(171): 5′ 171 nt homology arm, exon 1 TRAC gene
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 21 - DNA, 161 bp, Homo sapiens
  misc_feature (1)..(161): 3′ 161 nt homology arm, exon 1 TRAC gene
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 22 - DNA, 2080 bp, Artificial Sequence
  Synthetic anti-CD19 CAR cassette including JeT promoter, intron, anti-CD19 CAR, SV40 sequence
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 23 - DNA, 2083 bp, Artificial Sequence
  Synthetic anti-BCMA CAR construct including JeT promoter, intron, anti-BCMA CAR construct, SV40 sequence
  misc_feature (1057)..(1057): n is a, c, g, or t
  misc_feature (1098)..(1098): n is a, c, g, or t
  misc_feature (1120)..(1120): n is a, c, g, or t
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggtcctgggtgttcctgttctttctgtccgtgaccaccggtgtccactctcaggtgcagctggtggagtctgggggaggcttggtaaagcctggggggtcccttagactctcctgtgcagcctctggattcacttccagtaccgcctggatgagctgggtccgccaggctccagggaaggggctggagtgggttggccgtattaaaagcaaaagtgatggtgggacaacagactacgctgcacccgtgaaaggcagattcaccatctcaagagatgattcaaaaaacacgctgtttctgcaaatgaacagcctgaaaaccgaggacacagccgtgtattactgtgccaagggaggcgggacctacggctactggggccagggaaccctggtcaccgtctcctccggcggcggcggcagcggtggcggtggctcaggtggtggtggttcttcctatgtgctgactcagcctgcctccgtgtctgggtctcctggacagtcagtcaccatctcctgcactggaaccagcagtgatggtggtggtcacacctatgtctcctggtaccaacagcacccaggcaaagcccccaaactcatgatttatgatgtcagtaatcggccctcatgggtttctaatcgcttctctggctccaagtctggcaacacggcctccctgaccatctctgggctccaggctgangacgaggctgattattactgcggctcatatacaagcagcgnctcttatgtcttcggaactggnaccaagctgaccgtcctggctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 24 - DNA, 183 bp, Homo sapiens
  misc_feature (1)..(183): 5′ homology arm, TRAC gene exon 3, 183 nt
tatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtc

SEQ ID NO: 25 - DNA, 140 bp, Homo sapiens
  misc_feature (1)..(140): 3′ homology arm, TRAC gene exon 3
agtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaa

SEQ ID NO: 26 - DNA, 20 bp, Homo sapiens
  misc_feature (1)..(20): Exon 3 target sequence (guide sequence)
ttcggaacccaatcactgac

SEQ ID NO: 27 - DNA, 2442 bp, Artificial Sequence
  Synthetic Entire donor DNA anti-CD38 CAR plus exon 3 HAs on both ends
tatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaa

SEQ ID NO: 28 - DNA, 21 bp, Artificial Sequence
  Synthetic Forward primer for generating donor fragment of SEQ ID NO27
  modified_base (1)..(2): Phosphorothioate linkage
  modified_base (2)..(3): Phosphorothioate linkage
  modified_base (2)..(2): 2′-O-methylated
  modified_base (3)..(4): Phosphorothioate linkage
  modified_base (4)..(5): 2′-O-methylated
tatgccacagaagctgcaagg

SEQ ID NO: 29 - DNA, 20 bp, Artificial Sequence
  Synthetic Reverse primer for generating donor fragment of SEQ ID NO27, the 5′-most nucleoside (T) has a 5′ phosphate
  misc_feature: 5′ phosphate
ttaggatgcacccagagacc

SEQ ID NO: 30 - DNA, 326 bp, Homo sapiens
  misc_feature (1)..(326): DNA PD-1 locus 5′ HA
ctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttctta

SEQ ID NO: 31 - DNA, 380 bp, Homo sapiens
  misc_feature (1)..(380): DNA PD-1 locus 3′ HA
ggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcagg

SEQ ID NO: 32 - DNA, 20 bp, Homo sapiens
  misc_feature (1)..(20): PD-1 target site
ggccaggatggttcttaggt

SEQ ID NO: 33 - DNA, 2825 bp, Artificial Sequence
  Synthetic Donor DNA fragment having CD38 cassette (SEQ ID NO2) flanked by PD-1 Has
ctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcagg

SEQ ID NO: 34 - DNA, 20 bp, Artificial Sequence
  Synthetic Forward primer for generating donor fragment of SEQ ID NO33, the 5′-most nucleoside (C) has a 5′ phosphate
  misc_feature: 5′ phosphate
ctccccatctcctctgtctc

SEQ ID NO: 35 - DNA, 21 bp, Artificial Sequence
  Synthetic Reverse primer for generating donor fragment of SEQ ID NO33, Phosphorothioate linkage between first and second, second and third, and third and fourth nucleosides; C at position 1, C at position 2, and G at position 4 are 2′-O- methylated
  modified_base (1)..(2): Phosphorothioate linkage
  modified_base (1)..(2): 2′-O-methylated
  modified_base (2)..(3): Phosphorothioate linkage
  modified_base (3)..(4): Phosphorothioate linkage
  modified_base (4)..(4): 2′-O-methylated
cctggacccgtcattctacag

SEQ ID NO: 36 - DNA, 20 bp, Homo sapiens
  misc_feature (1)..(20): Forward primer for generating donor DNA PCR fragment from pAAV anti-CD38 CAR-TRAC construct (660 & 650 HAs)
tggagctagggcaccatatt

SEQ ID NO: 37 - DNA, 2415 bp, Artificial Sequence
  Synthetic anti-BCMA CAR construct donor fragment sequence, including JeT promoter, intron, anti-BCMA CAR construct, SV40 sequence, with 5′ and 3′ TRAC gene exon 1 homology arms
  misc_feature (1228)..(1228): n is a, c, g, or t
  misc_feature (1269)..(1269): n is a, c, g, or t
  misc_feature (1291)..(1291): n is a, c, g, or t
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggtcctgggtgttcctgttctttctgtccgtgaccaccggtgtccactctcaggtgcagctggtggagtctgggggaggcttggtaaagcctggggggtcccttagactctcctgtgcagcctctggattcacttccagtaccgcctggatgagctgggtccgccaggctccagggaaggggctggagtgggttggccgtattaaaagcaaaagtgatggtgggacaacagactacgctgcacccgtgaaaggcagattcaccatctcaagagatgattcaaaaaacacgctgtttctgcaaatgaacagcctgaaaaccgaggacacagccgtgtattactgtgccaagggaggcgggacctacggctactggggccagggaaccctggtcaccgtctcctccggcggcggcggcagcggtggcggtggctcaggtggtggtggttcttcctatgtgctgactcagcctgcctccgtgtctgggtctcctggacagtcagtcaccatctcctgcactggaaccagcagtgatggtggtggtcacacctatgtctcctggtaccaacagcacccaggcaaagcccccaaactcatgatttatgatgtcagtaatcggccctcatgggtttctaatcgcttctctggctccaagtctggcaacacggcctccctgaccatctctgggctccaggctgangacgaggctgattattactgcggctcatatacaagcagcgnctcttatgtcttcggaactggnaccaagctgaccgtcctggctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 38 - DNA, 2217 bp, Artificial Sequence
  Synthetic anti-CD19 CAR cassette including JeT promoter, intron, anti-CD19 CAR, SV40 sequence flanked by 5′ and 3′ TRAC gene exon 1 homology arms of SEQ ID NO20 and SEQ ID NO 21
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 39 - DNA, 1001 bp, Artificial Sequence
  Synthetic Sequenced PCR product of 5′ end of donor DNA plus adjacent TRAC gene exon 1 genomic sequence that included portion of anti-CD38 CAR construct and 660 nt homology arm
  misc_feature (1)..(15): n is a, c, g, or t
  misc_feature (18)..(19): n is a, c, g, or t
  misc_feature (790)..(792): n is a, c, g, or t
  misc_feature (925)..(925): n is a, c, g, or t
  misc_feature (931)..(931): n is a, c, g, or t
  misc_feature (933)..(934): n is a, c, g, or t
  misc_feature (1001)..(1001): n is a, c, g, or t
nnnnnnnnnnnnnnngcnngactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggnnngagatcatgtcctaaccctgatcctcttgtcccacagaattcgggcggagttagggcggagccaatcagcgtgcgccgttccgaaagttgccttttatggctgggcggagaatgggcggtgaacgccgatgantatatnannacgcgccgggtgtggcacagctagttccgtcgcagccgggatttgggtcgcggttcttgtttgtggn

SEQ ID NO: 40 - DNA, 1015 bp, Artificial Sequence
  Synthetic Sequenced PCR product of 3′ end of donor DNA plus adjacent TRAC gene exon 1 genomic sequence that included portion of anti-CD38 CAR construct and 650 nt homology arm
  misc_feature (1)..(16): n is a, c, g, or t
  misc_feature (988)..(988): n is a, c, g, or t
nnnnnnnnnnnnnnnnttctgctctacctgggagtggactccggtggtcacgctgagaaaaaagagaaagacccatgaccattccatggtggcggcctcgaggcctgtcaggagaggaaagagaagaaggttagtacaattgtctagatgcattcctagggctgcagggttcatagtgccacttttcctgcactgccccatctcctgcccaccctttcccaggcatagacagtcagtgacttactgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaacctgaaatgcagggctacttaataatactgcttattcttttatttaataggatcttcttcaaaaccccagcaatataactctggcagagtaaaggcaggcatgggaaaaaggcccagcaaagcaaactgtacatcttggaatctggagtggtctccccaacttaggctgggcattagcagaatgggaggtttatggtatgttggcattaagttgggaaatctatcacattaccaggagattgctctctcattgatagaggttttgaactataaatcanaacacctgcgtctaagccccagcgcta

SEQ ID NO: 41 - DNA, 1035 bp, Artificial Sequence
  Synthetic Sequenced PCR product of 5′ end of donor DNA plus adjacent TRAC gene exon 3 genomic sequence that included portion of the anti-CD38 CAR construct and 660 nt homology arm Exon3 5HA F sequence results
  misc_feature (2)..(21): n is a, c, g, or t
  misc_feature (31)..(31): n is a, c, g, or t
  misc_feature (1030)..(1030): n is a, c, g, or t
  misc_feature (1032)..(1035): n is a, c, g, or t
cnnnnnnnnnnnnnnnnnnnnctttgaggangagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccntnnnn

SEQ ID NO: 42 - DNA, 1030 bp, Artificial Sequence
  Synthetic Sequenced PCR product of 3′ end of donor DNA plus adjacent genomic TRAC gene exon 3 sequence that included portion of the anti-CD38 CAR construct and 650 nt homology arm
  misc_feature (1)..(18): n is a, c, g, or t
  misc_feature (952)..(952): n is a, c, g, or t
  misc_feature (1007)..(1007): n is a, c, g, or t
  misc_feature (1030)..(1030): n is a, c, g, or t
nnnnnnnnnnnnnnnnnngctgcacaggagagtctcagggaccctccaggcttgaccaagcctcccccagactccaccagctgcacctgagagtggacaccggtggtcacggacagaaagaacaggaacacccaggaccactccatggtggcggcctcgaggcctgtcaggagaggaaagagaagaaggttagtacaattgtctagatgcattcctagggctgcagggttcatagtgccacttttcctgcactgccccatctcctgcccaccctttcccaggcatagacagtcagtgacttactgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattcgacaggttttgaaagtttaggttcgtatctgtaaaaccaagaggccacagcggttatgggcttgcatgtcccaggcctgcatggcctgcaggacacacccacggttttccccaaggcagagcaggctgggtgggggaggcctgcagctcctgcacctcctgtcccttgcagcttctgtgcatatccccttacaaacagattctgtgcattcttccagggccaacagtattgtgtttcaagacaggaaacaccgactagacttggctcttattttatttctgagcccttctggggatggggtaggtgctcagaatttatcatcacaatacctcgggtccagatgggccccttagtgagttccgctaccacaggaaggatgactcaataagaacaactcaaatggaaatgaatatgggcccagggtgggtgtgaggagtggcaggggattcttganagacaggaccattgcccacagcctatgtgagtactgttgcttgtcttgaaagaangcaaaacctcttggctgtcctcn

SEQ ID NO: 43 - DNA, 1015 bp, Artificial Sequence
  Synthetic Sequenced PCR product of 3′ end of donor DNA plus adjacent PD-1 genomic sequence that included portion of the anti-CD38 CAR construct and 660 nt homology arm
  misc_feature (1)..(19): n is a, c, g, or t
  misc_feature (22)..(22): n is a, c, g, or t
  misc_feature (32)..(32): n is a, c, g, or t
  misc_feature (925)..(926): n is a, c, g, or t
  misc_feature (930)..(930): n is a, c, g, or t
  misc_feature (935)..(936): n is a, c, g, or t
  misc_feature (943)..(943): n is a, c, g, or t
  misc_feature (947)..(947): n is a, c, g, or t
  misc_feature (960)..(961): n is a, c, g, or t
  misc_feature (971)..(971): n is a, c, g, or t
  misc_feature (974)..(976): n is a, c, g, or t
  misc_feature (978)..(978): n is a, c, g, or t
  misc_feature (983)..(985): n is a, c, g, or t
  misc_feature (990)..(991): n is a, c, g, or t
  misc_feature (999)..(999): n is a, c, g, or t
  misc_feature (1012)..(1013): n is a, c, g, or t
  misc_feature (1015)..(1015): n is a, c, g, or t
nnnnnnnnnnnnnnnnnnntcntgctggtganggtggcctttattattttctgggttcgatctaagcgaagccgcttgttgcattctgactacatgaatatgacgccaagacggccagggccaacaagaaagcattaccaaccgtacgcccccccgcgagactacgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcaaactcctgggttcctctctggnncccnactcnncccctgncccnagtcccctctttnntcctgggcangcnnnanctctnnnccctnncagccggnccttggggctgcnngn

SEQ ID NO: 44 - DNA, 660 bp, Homo sapiens
  misc_feature (1)..(660): 5′ homology arm from exon 1 of TRAC gene, 660 nt
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 45 - DNA, 650 bp, Homo sapiens
  misc_feature (1)..(650): 3′ homology arm from exon 1 of TRAC gene, 650 nt
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 46 - DNA, 63 bp, Artificial Sequence
  Synthetic Encodes T2A peptide sequence of Thosea asigna virus
ggaagcggagagggcagaggaagtctgctaacatgcggtgacgtcgaggagaatcctggacct

SEQ ID NO: 47 - DNA, 122 bp, Artificial Sequence
  Synthetic SV40 polyA addition sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 48 - DNA, 2652 bp, Artificial Sequence
  Synthetic CD38 DAR insert 2652 nt
  misc_feature (2467)..(2467): n is a, c, g, or t
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaggtgcagctggtggagtccggaggcggcctggtgaaacctggcggatccctgaggctgtcctgcgccgctagcggattcaccttcagcgacgactacatgagctggatcaggcaggctcccggaaagggcctggagtgggtcgctagcgtgagcaatggccggcccacaacctactatgccgactccgtgcggggcaggtttaccatctccagggataacgctaagaactccctgtacctgcagatgaacagcctgcgggccgaagataccgccgtctactattgcgccagggaggattggggcggcgagttcacagactggggaaggggcaccctggtgaccgtgagcagcgcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacaccaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagtccgttctgacccagcctccttccgcctctggcacatccggccagagagtgaccatctcctgcagcggctccagctctaacatcggcatcaatttcgtgtactggtatcagcacctgccaggcacagctcccaagctgctgatctacaagaacaatcagaggccttccggcgtgccagaccggttctctggctccaagagcggcaactctgcctccctggctatctccggcctgcgcagcgaggacgaggctgattactattgcgccgcttgggacgatagcctgtctggctacgtgttcggcagcggcacaaaggtgaccgtgctgggacagccaaaggctgctccttctgtgacactgtttcccccttccagcgaggagctgcaggccaataaggccaccctggtgtgcctgatcagcgacttctatcctggagctgtgaccgtggcttggaaggctgattcttccccagtgaaggctggcgtggagacaacaacccccagcaagcagtctaacaataagtacgccgctagctcttatctgtctctgaccccagagcagtggaagtcccataggtcctatagctgtcangtcacccacgaagggagcacagtcgaaaaaaccgtcgcaccaaccgagtgttcctgataagttaacttgtttattgcagcttataatggttacaaataaagcaatagcatcacaaatttcacaaataaagcatttttttcactgcattctagttgtggtttgtccaaactcatcaatgtatctta

SEQ ID NO: 49 - DNA, 2984 bp, Artificial Sequence
  Synthetic CD38 DAR insert 2652 nt flanked by 5′ 171 nt and 3′ 161 nt TRAC exon 1 homology arms
  misc_feature (2638)..(2638): n is a, c, g, or t
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaggtgcagctggtggagtccggaggcggcctggtgaaacctggcggatccctgaggctgtcctgcgccgctagcggattcaccttcagcgacgactacatgagctggatcaggcaggctcccggaaagggcctggagtgggtcgctagcgtgagcaatggccggcccacaacctactatgccgactccgtgcggggcaggtttaccatctccagggataacgctaagaactccctgtacctgcagatgaacagcctgcgggccgaagataccgccgtctactattgcgccagggaggattggggcggcgagttcacagactggggaaggggcaccctggtgaccgtgagcagcgcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacaccaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagtccgttctgacccagcctccttccgcctctggcacatccggccagagagtgaccatctcctgcagcggctccagctctaacatcggcatcaatttcgtgtactggtatcagcacctgccaggcacagctcccaagctgctgatctacaagaacaatcagaggccttccggcgtgccagaccggttctctggctccaagagcggcaactctgcctccctggctatctccggcctgcgcagcgaggacgaggctgattactattgcgccgcttgggacgatagcctgtctggctacgtgttcggcagcggcacaaaggtgaccgtgctgggacagccaaaggctgctccttctgtgacactgtttcccccttccagcgaggagctgcaggccaataaggccaccctggtgtgcctgatcagcgacttctatcctggagctgtgaccgtggcttggaaggctgattcttccccagtgaaggctggcgtggagacaacaacccccagcaagcagtctaacaataagtacgccgctagctcttatctgtctctgaccccagagcagtggaagtcccataggtcctatagctgtcangtcacccacgaagggagcacagtcgaaaaaaccgtcgcaccaaccgagtgttcctgataagttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 50 - DNA, 645 bp, Artificial Sequence
  Synthetic 5′ exon 1 TRAC gene homology flanking sequence, 645 bp
tgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccct

SEQ ID NO: 51 - DNA, 600 bp, Artificial Sequence
  Synthetic 3′ exon 1 TRAC gene homology flanking sequence, exon 1 TRAC gene 600bp
gtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttattt

SEQ ID NO: 52 - DNA, 21 bp, Artificial Sequence
  Synthetic Cas12a target site in exon 1 of the TRAC gene
gagtctctcagctggtacacg

SEQ ID NO: 53 - DNA, 4497 bp, Artificial Sequence
  Synthetic Anti-CD38 DAR construct flanked by 645 bp and 600 bp homology sequences from TRAC locus
  misc_feature (3112)..(3112): n is a, c, g, or t
tgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaggtgcagctggtggagtccggaggcggcctggtgaaacctggcggatccctgaggctgtcctgcgccgctagcggattcaccttcagcgacgactacatgagctggatcaggcaggctcccggaaagggcctggagtgggtcgctagcgtgagcaatggccggcccacaacctactatgccgactccgtgcggggcaggtttaccatctccagggataacgctaagaactccctgtacctgcagatgaacagcctgcgggccgaagataccgccgtctactattgcgccagggaggattggggcggcgagttcacagactggggaaggggcaccctggtgaccgtgagcagcgcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacaccaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagtccgttctgacccagcctccttccgcctctggcacatccggccagagagtgaccatctcctgcagcggctccagctctaacatcggcatcaatttcgtgtactggtatcagcacctgccaggcacagctcccaagctgctgatctacaagaacaatcagaggccttccggcgtgccagaccggttctctggctccaagagcggcaactctgcctccctggctatctccggcctgcgcagcgaggacgaggctgattactattgcgccgcttgggacgatagcctgtctggctacgtgttcggcagcggcacaaaggtgaccgtgctgggacagccaaaggctgctccttctgtgacactgtttcccccttccagcgaggagctgcaggccaataaggccaccctggtgtgcctgatcagcgacttctatcctggagctgtgaccgtggcttggaaggctgattcttccccagtgaaggctggcgtggagacaacaacccccagcaagcagtctaacaataagtacgccgctagctcttatctgtctctgaccccagagcagtggaagtcccataggtcctatagctgtcangtcacccacgaagggagcacagtcgaaaaaaccgtcgcaccaaccgagtgttcctgataagttaacttgtttattgcagcttataatggttacaaataaagcaatagcatcacaaatttcacaaataaagcatttttttcactgcattctagttgtggtttgtccaaactcatcaatgtatcttagtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttgtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttattt

SEQ ID NO: 54 - DNA, 21 bp, Artificial Sequence
  Synthetic Reverse primer for producing donor DNA for insertion into TRAC exon 1 using cas12a
  modified_base (1)..(2): Phosphorothioate linkage
  modified_base (1)..(3): 2′-O-methylated
  modified_base (2)..(3): Phosphorothioate linkage
  modified_base (3)..(4): Phosphorothioate linkage
gcactgttgctcttgaagtcc

SEQ ID NO: 55 - DNA, 192 bp, Artificial Sequence
  Synthetic PCR synthesized 5′ homology arm, 192 nts
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccct

SEQ ID NO: 56 - DNA, 159 bp, Artificial Sequence
  Synthetic PCR synthesized 3′ homology arm, 159 nts
gtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgc

SEQ ID NO: 57 - DNA, 3003 bp, Artificial Sequence
  Synthetic Anti-CD38 DAR donor DNA, 30003 nucleotides
  misc_feature (2659)..(2659): n is a, c, g, or t
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaggtgcagctggtggagtccggaggcggcctggtgaaacctggcggatccctgaggctgtcctgcgccgctagcggattcaccttcagcgacgactacatgagctggatcaggcaggctcccggaaagggcctggagtgggtcgctagcgtgagcaatggccggcccacaacctactatgccgactccgtgcggggcaggtttaccatctccagggataacgctaagaactccctgtacctgcagatgaacagcctgcgggccgaagataccgccgtctactattgcgccagggaggattggggcggcgagttcacagactggggaaggggcaccctggtgaccgtgagcagcgcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacaccaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagtccgttctgacccagcctccttccgcctctggcacatccggccagagagtgaccatctcctgcagcggctccagctctaacatcggcatcaatttcgtgtactggtatcagcacctgccaggcacagctcccaagctgctgatctacaagaacaatcagaggccttccggcgtgccagaccggttctctggctccaagagcggcaactctgcctccctggctatctccggcctgcgcagcgaggacgaggctgattactattgcgccgcttgggacgatagcctgtctggctacgtgttcggcagcggcacaaaggtgaccgtgctgggacagccaaaggctgctccttctgtgacactgtttcccccttccagcgaggagctgcaggccaataaggccaccctggtgtgcctgatcagcgacttctatcctggagctgtgaccgtggcttggaaggctgattcttccccagtgaaggctggcgtggagacaacaacccccagcaagcagtctaacaataagtacgccgctagctcttatctgtctctgaccccagagcagtggaagtcccataggtcctatagctgtcangtcacccacgaagggagcacagtcgaaaaaaccgtcgcaccaaccgagtgttcctgataagttaacttgtttattgcagcttataatggttacaaataaagcaatagcatcacaaatttcacaaataaagcatttttttcactgcattctagttgtggtttgtccaaactcatcaatgtatcttagtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgc

SEQ ID NO: 58 - DNA, 645 bp, Homo sapiens
  misc_feature (1)..(645): 5′HA  645bp, Tim3 locus
gttaggagagcctccctttgttgatgaacaagcaagtagcccagatgggcgggccgtttcctggctgaccatgactaattttctgattgtctgtttccatcagccctgttctcccgtgttcacagaattgggccacaattctctcctagggcagtgtttctgaaagtgaggttctgagaccagccacttcagcaacacttgagaacttgttagaaataaaagttctcaggctctaccacaggccaactgagtcaggaactctagcagttgagcccagcaatctgtgttttcgcaaggcttcccagtgattctgatggccttcacatctgagaagcattgtcacagcgaatcatcctccaaacaggactgcagcagtagcttcctctttattctgtaagacatggcttgcagttttcctgaaatggagtaacctcactcaccgcttgagtcttggctctccttctctctctatgcagggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaa

SEQ ID NO: 59 - DNA, 600 bp, Homo sapiens
  misc_feature (1)..(600): 3′HA 600bp, Tim3 locus
ggacatccagatactggcttcttggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgccatctttatgaataagatttatctgtggatcatattaaaggtactgattgttctcatctctgacttccctaattatagccctggaggagggccactaagacctaaagtttaacaggccccattggtgatgctcagtgatatttaacaccttctctctgttttaaaactcatgggtgtgcctgggcgtggtggctcacacctctaatcccagcactttgggaggctgaggccggtggatcatgaggtcaggaattcgagaccagcctggccaacatagtaaaaccttgtctccactaaaaatacaaaaaattagccaggcatggttacgggagcctgtaattctagctacttggggggctgaagcaggagaatcacttgaacctgggagtcggaggttgtggtaagccaagatctcgcc

SEQ ID NO: 60 - DNA, 21 bp, Homo sapiens
  misc_feature (1)..(21): TIM-3 cas12a target site
gccagtatctggatgtccaat

SEQ ID NO: 61 - DNA, 20 bp, Homo sapiens
  misc_feature: 5′ phosphate
  misc_feature (1)..(20): TIM3 Forward primer for making donor DNA
tggaatacagagcggaggtc

SEQ ID NO: 62 - DNA, 20 bp, Homo sapiens
  modified_base (1)..(2): Phosphorothioate linkage
  modified_base (1)..(3): 2′-O-methylated
  misc_feature (1)..(20): TIM3 Reverse primer for making donor DNA
  modified_base (2)..(3): Phosphorothioate linkage
  modified_base (3)..(4): Phosphorothioate linkage
gcatgcaaatgtccactcac

SEQ ID NO: 63 - DNA, 2991 bp, Artificial Sequence
  Synthetic TIM3 donor DNA
  misc_feature (2624)..(2624): n is a, c, g, or t
tggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaggtgcagctggtggagtccggaggcggcctggtgaaacctggcggatccctgaggctgtcctgcgccgctagcggattcaccttcagcgacgactacatgagctggatcaggcaggctcccggaaagggcctggagtgggtcgctagcgtgagcaatggccggcccacaacctactatgccgactccgtgcggggcaggtttaccatctccagggataacgctaagaactccctgtacctgcagatgaacagcctgcgggccgaagataccgccgtctactattgcgccagggaggattggggcggcgagttcacagactggggaaggggcaccctggtgaccgtgagcagcgcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacaccaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagtccgttctgacccagcctccttccgcctctggcacatccggccagagagtgaccatctcctgcagcggctccagctctaacatcggcatcaatttcgtgtactggtatcagcacctgccaggcacagctcccaagctgctgatctacaagaacaatcagaggccttccggcgtgccagaccggttctctggctccaagagcggcaactctgcctccctggctatctccggcctgcgcagcgaggacgaggctgattactattgcgccgcttgggacgatagcctgtctggctacgtgttcggcagcggcacaaaggtgaccgtgctgggacagccaaaggctgctccttctgtgacactgtttcccccttccagcgaggagctgcaggccaataaggccaccctggtgtgcctgatcagcgacttctatcctggagctgtgaccgtggcttggaaggctgattcttccccagtgaaggctggcgtggagacaacaacccccagcaagcagtctaacaataagtacgccgctagctcttatctgtctctgaccccagagcagtggaagtcccataggtcctatagctgtcangtcacccacgaagggagcacagtcgaaaaaaccgtcgcaccaaccgagtgttcctgataagttaacttgtttattgcagcttataatggttacaaataaagcaatagcatcacaaatttcacaaataaagcatttttttcactgcattctagttgtggtttgtccaaactcatcaatgtatcttaggacatccagatactggcttcttggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgc

SEQ ID NO: 64 - DNA, 20 bp, Artificial Sequence
  Synthetic forward primer for sequencing across 5′ homology arm of anti-CD38 CAR in TRAC exon 3 locus
ctcctgaatccctctcacca

SEQ ID NO: 65 - DNA, 20 bp, Artificial Sequence
  Synthetic reverse primer for sequencing across 5′ homology arm of anti-CD38 CAR in TRAC exon 3 locus
gcggatccagctcatgtagt

SEQ ID NO: 66 - DNA, 20 bp, Artificial Sequence
  Synthetic forward primer for sequencing across 3′ homology arm of anti-CD38 CAR in TRAC exon 3 locus
cgttctgggtactcgtggtt

SEQ ID NO: 67 - DNA, 20 bp, Artificial Sequence
  Synthetic reverse primer for sequencing across 3′ homology arm of anti-CD38 CAR in TRAC exon 3 locus
ggagcacaggctgtcttaca

SEQ ID NO: 68 - DNA, 19 bp, Artificial Sequence
  Synthetic forward primer for sequencing across 5′ homology arm of anti-CD38 CAR in PD-1 locus
gtgtgaggccatccacaag

SEQ ID NO: 69 - DNA, 19 bp, Artificial Sequence
  Synthetic reverse primer for sequencing across 5′ homology arm of anti-CD38 CAR in PD-1 locus
acacacttgcgacccattc

SEQ ID NO: 70 - DNA, 20 bp, Artificial Sequence
  Synthetic forward primer for sequencing across 3′ homology arm of anti-CD38 CAR in PD-1  locus
cgttctgggtactcgtggtt

SEQ ID NO: 71 - DNA, 19 bp, Artificial Sequence
  Synthetic reverse primer for sequencing across 3′ homology arm of anti-CD38 CAR in PD-1 locus
gggactgtcttaggcttgg

SEQ ID NO: 72 - DNA, 20 bp, Artificial Sequence
  Synthetic forward primer for sequencing across 5′ homology arm of anti-CD38 DAR in TRAC exon 1 locus
cctgctttctgagggtgaag

SEQ ID NO: 73 - DNA, 20 bp, Artificial Sequence
  Synthetic reverse primer for sequencing across 5′ homology arm of anti-CD38 DAR in TRAC exon 1 locus
cagctcatgtagtcgtcgct

SEQ ID NO: 74 - DNA, 20 bp, Artificial Sequence
  Synthetic forward primer for sequencing across 3′ homology arm of anti-CD38 DAR in TRAC exon 1 locus
ggaatgaaaggggagaggag

SEQ ID NO: 75 - DNA, 20 bp, Artificial Sequence
  Synthetic reverse primer for sequencing across 3′ homology arm of anti-CD38 DAR in TRAC exon 1 locus
gagagcccttccctgacttt

SEQ ID NO: 76 - DNA, 20 bp, Artificial Sequence
  Synthetic forward primer for sequencing across 5′ homology arm of anti-CD38 DAR in TRAC exon 1 locus at Cas12a target site
cctgctttctgagggtgaag

SEQ ID NO: 77 - DNA, 20 bp, Artificial Sequence
  Synthetic reverse primer for sequencing across 5′ homology arm of anti-CD38 DAR in TRAC exon 1 locus at Cas12a target site
cagctcatgtagtcgtcgct

SEQ ID NO: 78 - DNA, 20 bp, Artificial Sequence
  Synthetic forward primer for sequencing across 3′ homology arm of anti-CD38 DAR in TRAC exon 1 locus at Cas12a target site
ggaatgaaaggggagaggag

SEQ ID NO: 79 - DNA, 20 bp, Artificial Sequence
  Synthetic reverse primer for sequencing across 3′ homology arm of anti-CD38 DAR in TRAC exon 1 locus
gagagcccttccctgacttt

SEQ ID NO: 80 - DNA, 21 bp, Artificial Sequence
  Synthetic GM-CSF cas12a target site
tacagaatgaaacagtagaag

SEQ ID NO: 81 - DNA, 2457 bp, Artificial Sequence
  Synthetic Anti-CD20 DAR construct
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaagtgcaattgcagcagcccggtgccgaactcgtgaaaccaggagcaagcgtaaagatgtcctgtaaggcatcaggttatacctttaccagctacaacatgcactgggtgaaacaaacgccggggcggggcctcgaatggataggcgcgatatatcccggaaatggcgataccagttacaatcagaagttcaaaggcaaagcgacactgacagctgataagtcttcaagcaccgcctatatgcaactttctagcctgaccagcgaagactccgccgtttattactgtgctcggtccacatactacggaggcgattggtactttaatgtgtggggtgcgggcaccactgtcactgtatcagcggcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacacccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagataaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagatagtcctgagccaatcaccggccatcttgtctgcctctcctggcgaaaaggtgacgatgacttgcagagccagtagctctgtaagctatatacactggttccagcaaaaaccgggctcttctccgaagccgtggatatacgcaacttcaaacctggcgtctggggttcctgtaaggtttagcggcagcggttcaggcacgagctacagccttactatctcccgggttgaggctgaagatgcagccacatactactgtcagcagtggacttcaaatccacctacattcgggggaggcacgaagctggagattaaacgaaccgttgcggcgcctagtgtgttcatattcccgccgtctgatgaacaactcaagtctggaacggcaagtgtggtgtgtctcctgaataatttttatcctagggaagcaaaggtgcagtggaaagtcgataacgcattgcaaagcggtaacagtcaagaatctgtaactgaacaagattctaaagattctacctacagtctctcctccacattgaccctgtcaaaagcagattatgagaagcacaaggtgtacgcatgtgaggtaacacatcaaggactcagcagcccagttacaaaaagtttcaatcgcggggaatgttgataagttaacttgtttattgcagcttataatggttacaaataaagcaatagcatcacaaatttcacaaataaagcatttttttcactgcattctagttgtggtttgtccaaactcatcaatgtatctta

SEQ ID NO: 82 - DNA, 20 bp, Artificial Sequence
  Synthetic forward primer for generating donor DNA, includes 5′ phosphate
  misc_feature: 5′ phosphate
atcacgagcagctggtttct

SEQ ID NO: 83 - DNA, 2808 bp, Artificial Sequence
  Synthetic Anti-CD20 DAR donor DNA including 5′ homology arm, anti-CD20 DAR construct, and 3′ homology arm
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggagctgggtgtttctgttcttcctctccgtcacaaccggcgtgcatagccaagtgcaattgcagcagcccggtgccgaactcgtgaaaccaggagcaagcgtaaagatgtcctgtaaggcatcaggttatacctttaccagctacaacatgcactgggtgaaacaaacgccggggcggggcctcgaatggataggcgcgatatatcccggaaatggcgataccagttacaatcagaagttcaaaggcaaagcgacactgacagctgataagtcttcaagcaccgcctatatgcaactttctagcctgaccagcgaagactccgccgtttattactgtgctcggtccacatactacggaggcgattggtactttaatgtgtggggtgcgggcaccactgtcactgtatcagcggcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacacccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagataaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagatagtcctgagccaatcaccggccatcttgtctgcctctcctggcgaaaaggtgacgatgacttgcagagccagtagctctgtaagctatatacactggttccagcaaaaaccgggctcttctccgaagccgtggatatacgcaacttcaaacctggcgtctggggttcctgtaaggtttagcggcagcggttcaggcacgagctacagccttactatctcccgggttgaggctgaagatgcagccacatactactgtcagcagtggacttcaaatccacctacattcgggggaggcacgaagctggagattaaacgaaccgttgcggcgcctagtgtgttcatattcccgccgtctgatgaacaactcaagtctggaacggcaagtgtggtgtgtctcctgaataatttttatcctagggaagcaaaggtgcagtggaaagtcgataacgcattgcaaagcggtaacagtcaagaatctgtaactgaacaagattctaaagattctacctacagtctctcctccacattgaccctgtcaaaagcagattatgagaagcacaaggtgtacgcatgtgaggtaacacatcaaggactcagcagcccagttacaaaaagtttcaatcgcggggaatgttgataagttaacttgtttattgcagcttataatggttacaaataaagcaatagcatcacaaatttcacaaataaagcatttttttcactgcattctagttgtggtttgtccaaactcatcaatgtatcttagtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgc

SEQ ID NO: 84 - DNA, 2077 bp, Artificial Sequence
  Synthetic Anti-CEA CAR construct
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaggtggctcaggatcgggtggatccggctctggtggctcaggatcggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccagtgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 85 - DNA, 2428 bp, Artificial Sequence
  Synthetic Anti-CD20 DAR donor DNA including 5′ homology arm, anti-CD20 DAR construct, and 3′ homology arm
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaggtggctcaggatcgggtggatccggctctggtggctcaggatcggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccagtgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgc

SEQ ID NO: 86 - DNA, 21 bp, Artificial Sequence
  Synthetic CD7 locus Cas12a target site
ctacgaggacggggtggtgcc

SEQ ID NO: 87 - DNA, 21 bp, Artificial Sequence
  Synthetic CD7 locus Cas12a alternate target site
cttctcagaggaacagtccca

SEQ ID NO: 88 - DNA, 20 bp, Artificial Sequence
  Synthetic forward primer producing donor fragment for insertion into CD7 locus
  modified_base (1)..(2): Phosphorothioate linkage
  modified_base (1)..(1): 2′-O-methylated
  modified_base (2)..(3): Phosphorothioate linkage
  modified_base (3)..(4): Phosphorothioate linkage
  modified_base (3)..(4): 2′-O-methylated
ctgcagggaggacattctct

SEQ ID NO: 89 - DNA, 19 bp, Artificial Sequence
  Synthetic reverse primer producing donor fragment for insertion into CD7 locus
  misc_feature: 5′ phosphate
ttccctactgtcaccagga

SEQ ID NO: 90 - DNA, 212 bp, Artificial Sequence
  Synthetic 5′ homology arm for CD7 locus insertion
ctgcagggaggacattctctgtccttctggccagactgatggtgacagcccaggtcctccccagaggtgcagcagtctccccactgcacgactgtccccgtgggagcctccgtcaacatcacctgctccaccagcgggggcctgcgtgggatctacctgaggcagctcgggccacagccccaagacatcatagtctacgaggacggggtggt

SEQ ID NO: 91 - DNA, 170 bp, Artificial Sequence
  Synthetic 3′ homology arm for CD7 locus insertion
ctacggacagacggttccggggccgcatcgacttctcagggtcccaggacaacctgactatcaccatgcaccgcctgcagctgtcggacactggcacctacacctgccaggccatcacggaggtcaatgtctacggctccggcaccctggtcctggtgacaggtagggaa